(19) United States
(12) Patent Application Publication
(10) Pub. No.: US 2026/0035705 A1
(43) Pub. Date: Feb. 5, 2026

(54) ENGINEERED MICROORGANISMS WITH G3P ->3PG ENZYME AND/OR FRUCTOSE-1,6-BISPHOSPHATASE INCLUDING THOSE HAVING SYNTHETIC OR ENHANCED METHYLOTROPHY

(71) Applicant: Genomatica, Inc., San Diego, CA (US)

(72) Inventors: Harish Nagarajan, San Diego, CA (US); Tae Hoon Yang, San Diego, CA (US); Ali Khodayari, San Diego, CA (US)

(21) Appl. No.: 19/043,192

(22) Filed: Jan. 31, 2025

Related U.S. Application Data
(62) Division of application No. 17/255,352, now Pat. No. 12,234,463.
(60) Provisional application No. 62/690,209, filed on Jun. 26, 2018.

(51) Int. Cl. C12N 15/52; C12N 9/02; C12N 9/16; C12N 9/88; C12N 9/90; C12P 5/00; C12P 5/02; C12P 7/16; C12P 7/18; C12P 7/24; C12P 7/42; C12P 7/46; C12P 7/6409; C12P 13/00

(52) U.S. Cl. CPC C12N 15/52; C12N 9/0008; C12N 9/16; C12N 9/88; C12N 9/90; C12P 5/007; C12P 5/026; C12P 7/16; C12P 7/18; C12P 7/24; C12P 7/42; C12P 7/46; C12P 7/6409; C12P 13/001; C12P 13/005; C12Y 102/01012; C12Y 301/03011; C12Y 401/02009; C12Y 401/02043; C12Y 503/01027

(57) ABSTRACT

Described herein are engineered cells including ones having synthetic methylotrophy which include an NADH-dependent enzyme capable of converting G3P to 3PG (e.g., B. methanolicus gapN) and/or fructose-1,6-bisphosphatase, along with hexulose-6-phosphate synthase, 6-phospho-3-hexuloisomerase, a phosphoketolase, or a combination thereof. Engineered cells of the disclosure beneficially maintain adequate pool sizes of phosphorylated C3 and/or C4 compounds, and/or provide increased levels of NADPH. As such, the modifications allow for the generation of C6 compounds from C1 (e.g., a methanol feedstod) and C5 compounds, the regeneration of C5 compounds from C6 compounds by carbon rearrangement, and an improved balance between regeneration of C5 compounds and lower glycolysis. In turn, this allows the engineered microorganism to generate sufficient quantities of metabolic precursors (e.g., acetyl-CoA) which can be used in a bioproduct pathway, and the engineered cells can include further modifications to those pathway enzymes allowing for production of a desired bioproduct.

### PRIORITY CLAIM

This application is a divisional of U.S. patent application Ser. No. 17/255,352, filed Dec. 22, 2020, which is a U.S. National Stage Entry of International Application No. PCT/US2019/039207, filed Jun. 26, 2019, which claims the benefit of U.S. Provisional Patent Application Ser. No. 62/690,209, filed Jun. 26, 2018, entitled Engineered Microorganisms with G3P→3PG Enzyme and/or Fructose-1,6-Bisphosphatase Including those Having Synthetic or Enhanced Methylotrophy, the disclosures of which are incorporated herein by reference in their entireties.

### REFERENCE TO SEQUENCE LWIN

This application contains a Sequence Listing in a computer readable form, submitted via USPTO Patent Center. The entire contents of the TXT file entitled “GN00088US2_Sequence_Listing.txt” created on Jun. 25, 2019, and having a size of 29.2 bytes, is incorporated herein by reference.

### FIELD OF INVENTION

The disclosure is directed to engineered microorganisms having synthetic or enhanced methylotrophy as well as engineered microorganisms that utilize a NADP-dependent glyceraldehyde-3-phosphate dehydrogenase and/or a fructose-1,6-bisphosphatase.

### BACKGROUND

Methylotrophy relates to the ability of a microorganism to use single carbon (C1) compounds, such as methanol and methane, as energy and carbon sources. Carbon feedstocks that include methane, which is available from natural gas, and it oxidation product, methanol, have become widely available and are now relatively inexpensive. Therefore, using C1 feedstocks has become attractive for fermentation technologies for the production of bioproducts, as compared to traditional feedstocks which typically use C6 or C5 sugars, which can be more costly.

Methylotrophic microorganisms are generally not favored for industrial processes as many have strict aerobic requirements, generate relatively few metabolic intermediates, and are thus far unsuitable for facile genetic engineering. Therefore, the development of synthetic methylotrophy in industrially useful microorganisms is challenging.

### SUMMARY

In some embodiments, the current invention provides engineered microorganisms having synthetic or improved methylotrophy, methods for producing a bioproduct using the engineered microorganisms, compositions that include the microorganisms, and also bioproduct-containing compositions prepared using the microorganisms. Engineered microorganisms of the invention include at least one modification relating to an enzyme(s) leading into the lower glycolysis (EMP) pathway (a NADP-dependent glyceraldehyde-3-phosphate dehydrogenase), and an enzyme(s) of the ribulose monophosphate (RuMP) cycle.

It has been found that native organisms, such as E. coli, which are incapable of performing methylotrophy, typically have a relatively high-flux through the EMP pathway. To achieve synthetic methylotrophy, engineered microorganisms of the disclosure utilize one or more enzyme(s) of the RuMP cycle and are also engineered to beneficially maintain an adequate pool size of one or more phosphorylated 3 and/or 4 carbon species (e.g., glyceraldehyde 3-phosphate (G3P), dihydroxyacetone phohsphate (DHAP), and erythrose 4-phosphate (E4P)). In one aspect of the disclosure, it has been discovered that this can be achieved in engineered organisms using an enzyme that converts glyceraldehyde 3-phosphate (G3P) to 3-phosphoglycerate (3PG), wherein the enzyme reduces NADP to NADPH (and also may be referred to as a non-phosphorylating enzyme as discussed herein), such as B. methanolicus GapN, or a functional equivalent thereof. In another aspect of the disclosure, this can be achieved in engineered organisms using a fructose-1,6-bisphosphatase, such as B. methanolicus GlpN, or a functional equivalent thereof.

Accordingly, an embodiment of the invention provides an engineered microorganism having synthetic or enhanced methylotrophy, the engineered microorganism including: (a) exogenous enzyme A that (ai) is capable of converting glyceraldehyde 3-phosphate (G3P) to 3-phosphoglycerate (3PG), that (aii) has at least 50% sequence identity to SEQ ID NO:1 (B. methanolicus gapN), wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii).

The modifications to the microorganism allows for the generation of C6 compounds from C1 and C5 compounds, the regeneration of C5 compounds from C6 compounds by carbon rearrangement, and an improved balance between regeneration of C5 compounds and lower glycolysis. In turn, this allows the engineered microorganism to generate sufficient quantities of metabolic precursors (e.g., acetyl-CoA) which can be used in a bioproduct pathway to produce a target compound, while at the same time providing good cell health for growth during fermentation methods.

In embodiments, the engineered microorganism can optionally include one or more modifications to native gene(s) of the organism's lower glycolysis pathway. For example, the modification can be one that attenuates or eliminates the organism's native NAD-dependent glyceraldehyde-3-phosphate dehydrogenase activity (GapA), native phosphoglycerate kinase activity (pgk. Along with the gapN modification, such deletions can enhance the pool size of the one or more phosphorylated 3 and/or 4 carbon species to provide cellular benefits as described herein.

In embodiments, the engineered microorganism can optionally include a NAD⁺-dependent methanol dehydrogenase (MDH), such as an exogenous MDH. The organism can have high MDH activity made possible by enhanced enzymatic activity, e.g., using a MDH variant, enhanced expression, or both. The MDH activity can provide an increased pool of C1 compound (formaldehyde), and the cell modifications can provide for generation of C5 compounds, for increased flux to the C6 compounds using the RuMP enzymes.

In embodiments, the engineered microorganism can optionally include one or more modifications to provide: (a) expression or increased activity of a transketolase(s) to increase the pool size of erythrose 4-phosphate, (b) expression or increased activity of sedoheptulose 1,7 bisphosphatase (SBPase or GlpX) to operate the SBPase variant of the RuMP cycle in the engineered organism; attenuates or eliminates the organism's native transaldolase activity (e.g., talB, talA, talC).

In embodiments, the disclosure provides an engineered microorganism comprising the following modifications:
I. (1) exogenous enzyme A (e.g., GapN), (2) exogenous Hps, (3) exogenous Phi, (4) exogenous MeDH, (5) exogenous GlpX, and (6) optionally exogenous phosphoketolase (PK);
II. optional modification causing deletion or attenuation of one or more of the following endogenous enzymes: (7) ATP-dependent 6-phosphofructokinase (Pfk), (8) ribulose-phosphate 3-epimerase (Rpe), (9) ribose-5-phosphate isomerase (Rpi), (10) transketolase (Tkt), (11) fructose-bisphosphate aldolase (Fba), (12) glucose-6-phosphate 1-dehydrogenase (Zwf), and optionally replacement of any of (7)-(11) with an exogenous counterpart enzyme, such as from Bacillus; and
III. optional modification causing deletion or attenuation of one or more of the following endogenous enzymes: (12) NAD-dependent glyceraldehyde-3-phosphate dehydrogenase (GapA), (13) transaldolase (e.g., TalB, TalA, and/or TalC), (14) phosphoglycerate kinase (Pgk), (15) phosphoglycerate mutase (Gpm), (16) enolase (Eno), (17) deoxyribose phosphate aldolase (DeoC), (18) methyl glyoxal synthase (MgsA), and (19) ATP-dependent 6-phosphofructokinase (pfkA and pfkB).

In other embodiments, the current invention provides engineered microorganisms (where synthetic or improved methylotrophy is not necessarily required) that include exogenous enzyme A that (ai) is capable of converting glyceraldehyde 3-phosphate (G3P) to 3-phosphoglycerate (3PG) or (aii) has at least 50% sequence identity to SEQ ID NO:1 (B. methanolicus GapN), wherein enzyme A is capable of reducing NADP to NADPH. Such a modification is useful in a metabolically engineered organism when a pathway to a desired chemical product utilizes NADPH as a redox source. In embodiments, the generation of NADPH can provide an advantage as a redox source in that fewer cellular reactions utilize NADPH, and in turn these electrons can be directed to the product pathway rather than other cellular reactions. Further, cellular ratios of NADPH to NADP+ can be higher than NADH to NAD+ ratios, which can provide an additional thermodynamic driving force for the product pathway. For example, an engineered microorganism that includes enzyme A (e.g., GapN or a functional equivalent thereof) can include a product pathway that utilizes increased cellular amounts of NADPH, such as amino acid pathways, particularly pathways to aromatic amino acids that require high levels of C3 and C4-phosphates such as G3P and E4P. Additionally the use of GapN with attenuated or deleted GapA can be used to improve the production of NADPH intensive products, such as 1,3 BDO.

In embodiments, the engineered microorganism can optionally include one or more transgene(s) encoding a protein of a metabolic pathway that promotes production of a target product or intermediate thereof, wherein the metabolic pathway uses a compound resulting from methanol consumption by the cell. In some embodiments, the one or more transgenes can be part of a pathway that forms an alcohol such as 1-butanol, isobutanol, 1,4-butanediol, or 1,3-butanediol. In some embodiments, the one or more transgenes can be part of a pathway that forms an acid or an acid ester such as methacrylic acid (MAA) or methyl methacrylate (MMA).

Other embodiments are directed to compositions including an engineered microorganism of the disclosure, such as cell culture compositions, and also compositions including one or more product(s) produced from the engineered microorganism. For example, a composition can include a target product produced by the engineered microorganisms, where the composition has been purified to remove cells or other components useful for cell culturing. The composition may be treated to enrich or purify the target product or intermediate thereof.

Other embodiments of the disclosure are directed to products made from the target product obtained from methods using the engineered microorganism.

### DESCRIPTION OF THE DRAWINGS

FIG. 1 illustrates the conversion of G3P to 3PG through 1,3-BPG (also referred to as 3-phosphoglyceroyl-P) using gapA and pgk (in a native/parental cell), as compared to the direct conversion of G3P to 3PG using gapN and NADP⁺ in a cell modified with exogenous gapN and having a gapA deletion. Reactions 1 and 2, native to E. coli, are marked as “parental”. Reaction 3, the GapN by-pass of the present invention where gapN is expressed with a deleted or otherwise attenuated gapA, is marked as “gapN with delta gapA”.

FIG. 2 illustrates a metabolic pathway leading from glyceraldehyde 3-phosphate (G3P) in a cell having gapA and pgk. Reaction 1 is catalyzed by a glyceraldehyde-3-phosphate dehydrogenase (GapA); Reaction 2 by a phosphoglycerate kinase (Pgk).

FIG. 3 illustrates a metabolic pathway leading from methanol to fructose-6-phosphate using MeDH, HPS, and PHI, in a cell having synthetic methylotrophy. Methanolₑₓ is external methanol, added as a carbon feed. Reaction 1 is catalyzed by a methanol dehydrogenase (mdh); Reaction 2 by a hexulose-6-phosphate synthase (hps); Reaction 3 by a phospho-3-hexuloisomerase.

FIG. 4 illustrates alternative formaldehyde assimilation pathways (RuMP and DHA) and metabolic intermediates from each that can be used in a product pathway. Enzymes catalyzing the reactions are (1) methanol dehydrogenase, e.g. EC 1.1.1, (2) hexulose-6-phosphate synthase, e.g. EC 4.1.2.43, (3) 6-phospho-3-hexuloisomerase, e.g. EC 5.3.1.27, (6) DHA (dihydroxyacetone) synthase, e.g. EC 2.2.1.3, (7) F6P (fructose-6-phosphate) aldolase, e.g. EC 4.1.2, (8) DHA kinase, e.g. EC 2.7.1.121, (9) fructose-bisphosphate aldolase, e.g. EC 4.1.2.13. The fusions described herein can catalyze two or more of the reactions.

FIG. 5 illustrates metabolic pathways leading to and from various 3, 4, 5, and 6 carbon metabolites including glyceraldehyde 3-phosphate (G3P), dihydroxyacetone-phosphate, erythrose 4-phospohate, ribose 5-phosphate, ribulose 5-phosphate, xylulose 5-phosphate, fructose 6-phosphate, and fructose 1,6-biphosphate in a cell having synthetic methylotrophy. Reaction 1 is catalyzed by a 6-phosphofructokinase (pfk, preferably pfkAB); Reaction 2 by a fructose-bisphosphate aldolase (fba, preferably fbaAB); Reaction 3 by a triose-phosphate isomerase (tpi, preferably tpiA); Reaction 4 by a transketolase (tkt, preferably tktAB); Reaction 5 by a transketolase (tkt, preferably tktAB); Reaction 6 a by ribose-5-phosphate isomerase (rpi, preferably rpiAB); Reaction 7 by a ribulose-phosphate 3-epimerase (rpe).

FIG. 6 illustrates metabolic pathways leading to and from, dihydroxyacetone-phosphate, erythrose 4-phospohate, sedoheptulose-1,7-biphosphate, and sedoheptulose-7-phosphate, in a cell having synthetic methylotrophy. Reaction 1 is catalyzed by a fructose-bisphosphate aldolase (fba); Reaction 2 by a fructose-1,6-bisphosphatase (glpX).

FIG. 7 illustrates an exemplary target product pathway, a 1,4-BDO product pathway, which can exploit acetyl-CoA available from methanol assimilation as disclosed herein. Enzymes catalyzing the biosynthetic reactions are: (1) succinyl-CoA synthetase; (2) CoA-independent succinic semialdehyde dehydrogenase; (3) α-ketoglutarate dehydrogenase; (4) glutamate:succinate semialdehyde transaminase; (5) glutamate decarboxylase; (6) CoA-dependent succinic semialdehyde dehydrogenase; (7) 4-hydroxybutanoate dehydrogenase; (8) α-ketoglutarate decarboxylase; (9) 4-hydroxybutyryl CoA: acetyl-CoA transferase; (10) butyrate kinase; (11) phosphotransbutyrylase; (12) aldehyde dehydrogenase; (13) alcohol dehydrogenase.

FIG. 8 illustrates exemplary target product pathways, 2-hydroxyisobutyrate and methacrylic acid from acetyl-CoA/methyl-methacrylate (MMA), which can exploit acetyl-CoA available from methanol assimilation as disclosed herein, 2-Hydroxyisobutyrate and methacrylic acid production can be carried out by the following enzymes: A) acetyl-CoA:acetyl-CoA acyltransferase, B) acetoacetyl-CoA reductase (ketone reducing), C) 3-hydroxybutyryl-CoA mutase, D) 2-hydroxyisobutyryl-CoA dehydratase, E) methacrylyl-CoA synthetase, hydrolase, or transferase, F) 2-hydroxyisobutyryl-CoA synthetase, hydrolase, or transferase.

FIG. 9 is a table listing SEQ ID NOs of corresponding proteins.

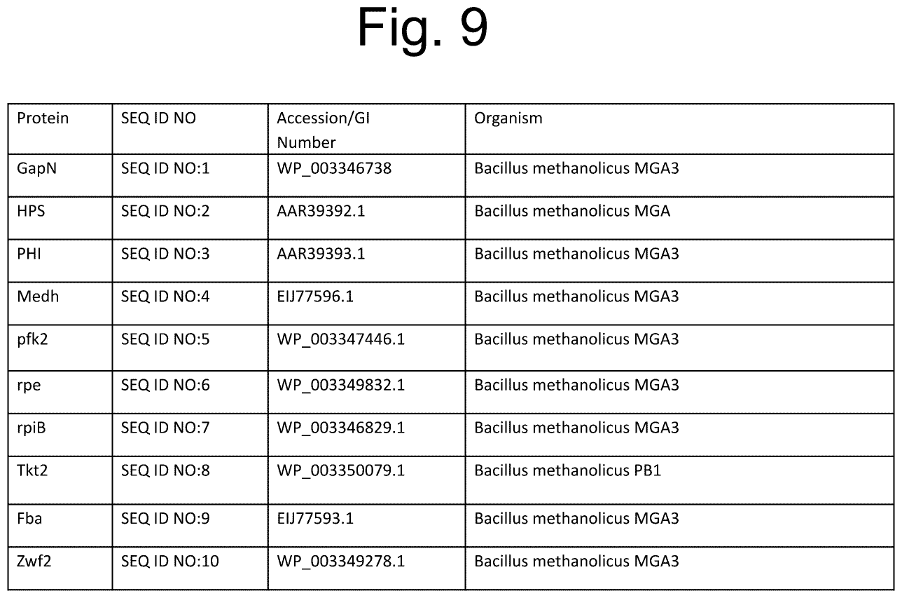

FIG. 10 demonstrates growth on methanol for a synthetic methylotroph and presents growth characteristics (optical density as OD at 600 nm; specific growth per hour). Panel A depicts growth data for control gapA expressing strain that demonstrates no growth on methanol. Panel B depicts growth data for gapN expressing strain showing growth on methanol. Panel C depicts growth rate on methanol for the gapN expressing strain. Dotted line is expected line for no growth (dilution). Solid line with black dots is the actual experimental data.

FIG. 11 demonstrates the affects of overexpression of enzymes Fba, Fba2, GlpX and Tkt on formaldehyde consumption (micromolar) at different cell densities. E. coli strains with additional GlpX expressed from a plasmid compared to the parent strain were analyzed for formaldehyde consumption in the absence of other carbon sources using a NASH assay. GlpX is Accession WP_003352248.1; Fba is Accession EU77616.1; Fba2 is Accession EU80286.1; Tkt is Accession. EU77615.1; and EV is control. Formaldehyde consumption, which is assimilated via the RuMP cycle enzymes is a proxy for measuring RuMP cycle activity. Optical density as OD at 600 nm is an indicator of cell density.

The embodiments of the description described herein are not intended to be exhaustive or to limit the disclosure to the precise forms disclosed in the following detailed description. Rather, the embodiments are chosen and described so that others skilled in the art can appreciate and understand the principles and practices of the description.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The term “synthetic methylotrophy” refers to the engineering in a microbial cell that allows it to utilize a C1 carbon (such as methanol or methane) as an energy source. A host cell in which the synthetic methylotrophy is engineered into otherwise is not able to grow on a C1 carbon source. Engineering a microorganism to provide synthetic methylotrophy will generally involve the expression of least one exogenous (i.e., “non-native”) enzyme in the cell, and more typically two or more, or three or more exogenous, enzymes in the cell. Other modifications can involve deletion of native genes or modification(s) that otherwise cause attenuation of native gene activity. The exogenous enzyme(s) can be incorporated into a natural metabolite pathway in the cell with one or more endogenous (i.e., “native”) enzymes, wherein exogenous enzyme(s) can convert a metabolite derived from 1C carbon into one that can be utilized by the cell's native enzymes, such as for cell growth or bioproduct production. Microorganisms that are non-methylotrophic and that can be engineered with exogenous enzyme(s) of the disclosure include E. coli and other prokaryotic and eukaryotic organisms as described herein.

The term “enhanced methylotrophy” refers to engineering a methylotrophic microbial cell that improves utilization of a 1C carbon (such as methanol or methane) as an energy source. A cell with “enhanced methylotrophy” can demonstrate enhanced cell growth or bioproduct production over the native methylotropic microbial cell. Microorganisms that are non-methylotropic and that can be engineered with exogenous enzyme(s) of the disclosure include E. coli and other prokaryotic and eukaryotic organisms as described herein.

Aspects of the current disclosure are directed to engineered microorganisms having at least one modification (i.e., “Enzyme A”) that affects the products leading into lower glycolysis (EMP) cycle. In one embodiment, Enzyme A is an enzyme that converts glyceraldehyde 3-phosphate to 3-phosphoglycerate (3PG), or is an enzyme that has at least 50% sequence identity to SEQ ID NO:1 (B. methanolicus gapN), wherein enzyme A is capable of reducing NADP to NADPH. In another embodiment, Enzyme A is a fructose-1,6-bisphosphatase, such as B. methanolicus GlpN, or a functional equivalent thereof.

The engineered microorganism can also have one or more exogenous enzyme(s) of the ribulose monophosphate (RuMP) cycle, (i.e., “Enzyme B”). The one or more RuMP cycle enzymes include, but is not limited to a hexulose-6-phosphate synthase, a 6-phospho-3-hexuloisomerase, a phosphoketolase, or any combination of these enzymes.

In preferred embodiments, the engineered microorganism also includes an exogenous methanol dehydrogenase, such as a methanol dehydrogenase that has one or more variant amino acids as compared to a wild type sequence.

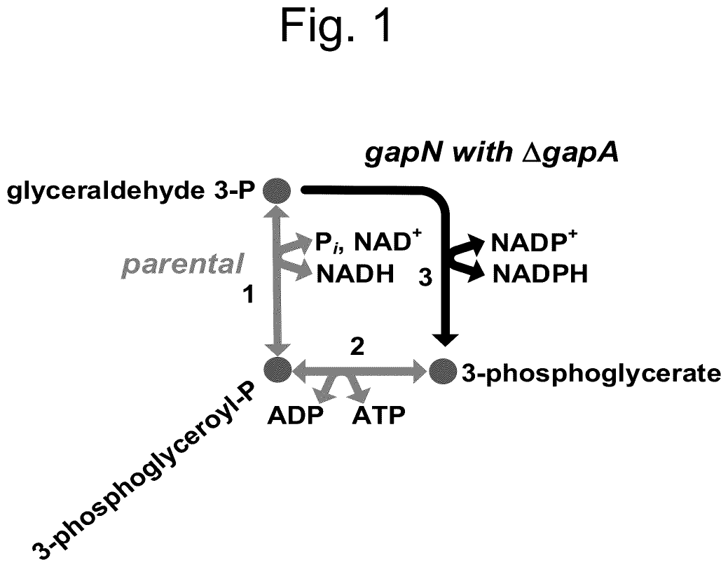

In some embodiments, the engineered microorganism includes an enzyme (“enzyme A”) that is capable of irreversibly converting glyceraldehyde 3-phosphate (G3P) to 3-phosphoglycerate (3PG) using the reduction of NADP+ to NADPH, or an enzyme that has at least 50% sequence identity to SEQ ID NO:1 (B. methanolicus gapN). Unlike E. coli gapA (glyceraldehyde-3-phosphate dehydrogenase A; P0A9B2) which uses the cofactor NAD to catalyze the oxidative phosphorylation of G3P to 13BPG, enzyme A of the current disclosure uses NADP to catalyze the conversion of G3P to 3PG (shown below). The conversion of G3P to 3PG, which utilizes NADP, appears to require no detectable amounts of phosphate for the conversion, and therefore may be referred to as a “non-phosphorylating” enzyme, which further distinguishes its enzymatic activity from E. coli gapA, which otherwise carriers out oxidative phosphorylation. Further, Enzyme A (e.g., GapN) converts G3P at a slower rate, based on comparing the conversion of G3P to 3PG by GapN, as compared to the conversion of G3P to 13BPG by E. coli GapA. FIG. 1 illustrates the conversion of G3P to 3PG through 1,3-BPG using gapA and pgk, as compared to the direct conversion of G3P to 3PG using gapN and NADP⁺.

For example, the enzyme kinetics of gapN are:

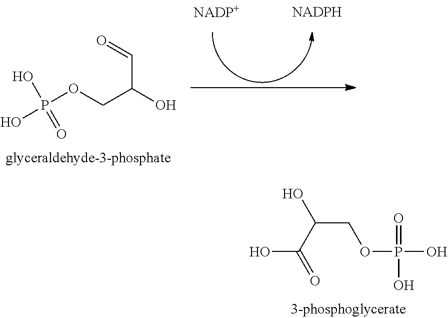 [SMILES: O=CC(O)COP(=O)(O)O ; O=C(O)C(O)COP(=O)(O)O]

One exemplary Enzyme A sequence is based on the Bacillus methanolicus NADP-dependent glyceraldehyde-3-phosphate dehydrogenase gapN (Genbank Accession number WP_003346738, 481 amino acids long; SEQ ID NO:1). The engineered microorganism of the disclosure can express an Enzyme A that has a sequence that is related Bacillus methanolicus gapN, such as a Bacillus methanolicus gapN homolog. For example, Enzyme A can have a sequence that has 50% or greater, 60% or greater, 70% or greater, 80% or greater, 85% or greater, 90% or greater, or 95% or greater, identity to SEQ ID NO: 1. Homologs of SEQ ID NO: 1 can be identified by sequence identity searching (e.g., algorithms well known to those skilled in the art, such as Align, BLAST, Clustal W, as described herein).

Exemplary Enzyme A sequences include those found in Table 1 below, which include many sequences from various Bacillus species.

TABLE 1

| | | % ID to |
|---|---|---|
| | | B. methanolicus |
| Accession | Organism | MGA3 |
| WP_003346738 | Bacillus methanolicus | 100 |
| WP_003351798 | Bacillus methanolicus | 95.01 |
| WP_026964094 | Alicyclobacillus pomorum | 68.75 |
| WP_089533800 | Virgibacillus necropolis | 66.527 |
| WP_011983786 | Bacillus cytotoxicus | 65.89 |
| WP_097894576 | Bacillus | 65.89 |
| WP_087097851 | Bacillus cytotoxicus | 65.678 |
| WP_068447188 | Lentibacillus amyloliquefaciens | 65.546 |
| WP_098359346 | Bacillus cereus | 65.466 |
| WP_098558080 | Bacillus cereus | 65.466 |
| EEL51944 | Bacillus cereus Rock3-44 | 65.254 |
| WP_000208150 | Bacillus cereus | 65.254 |
| WP_088038585 | Bacillus mycoides | 65.254 |
| WP_000213646 | Bacillus cereus group | 65.042 |
| WP_000213650 | Bacillus cereus group | 65.042 |
| WP_002010835 | Bacillus | 65.042 |
| WP_002125495 | Bacillus cereus group | 65.042 |
| WP_002134606 | Bacillus cereus | 65.042 |
| WP_002140513 | Bacillus cereus | 65.042 |
| WP_016093244 | Bacillus cereus | 65.042 |
| WP_017153122 | Bacillus bingmayongensis | 65.042 |
| WP_025150283 | Bacillus sp. H1a | 65.042 |
| WP_070141032 | Bacillus cereus group | 65.042 |
| WP_078178206 | Bacillus mycoides | 65.042 |
| WP_088035237 | Bacillus thuringiensis | 65.042 |
| WP_088106515 | Bacillus cereus | 65.042 |
| WP_088292461 | Bacillus mycoides | 65.042 |
| WP_105585763 | Bacillus sp. MYb209 | 65.042 |
| WP_105989975 | Bacillus sp. M21 | 65.042 |
| WP_077328816 | Virgibacillus siamensis | 64.916 |
| ABK84147 | Bacillus thuringiensis str. | 64.831 |
| | Al Hakam | |
| WP_000213613 | Bacillus | 64.831 |
| WP_000213620 | Bacillus cereus | 64.831 |
| WP_000213631 | Bacillus | 64.831 |
| WP_000213637 | Bacillus | 64.831 |
| WP_000213642 | Bacillus cereus | 64.831 |
| WP_000213643 | Bacillus thuringiensis | 64.831 |
| WP_002063972 | Bacillus cereus | 64.831 |
| WP_002087376 | Bacillus cereus | 64.831 |
| WP_003205982 | Bacillus cereus group | 64.831 |
| WP_016113266 | Bacillus cereus group | 64.831 |
| WP_018783531 | Bacillus | 64.831 |
| WP_048373546 | Bacillus sp. LK2 | 64.831 |
| WP_052943462 | Bacillus thuringiensis | 64.831 |
| WP_070169862 | Bacillus mycoides | 64.831 |
| WP_074615306 | Bacillus cereus | 64.831 |
| WP_076869997 | Bacillus cereus | 64.831 |
| WP_078985830 | Bacillus anthracis | 64.831 |
| WP_086388821 | Bacillus thuringiensis | 64.831 |
| WP_097786660 | Bacillus pseudomycoides | 64.831 |
| WP_097793698 | Bacillus pseudomycoides | 64.831 |
| WP_097955132 | Bacillus toyonensis | 64.831 |
| WP_098116852 | Bacillus pseudomycoides | 64.831 |
| WP_098162831 | Bacillus toyonensis | 64.831 |
| WP_098335345 | Bacillus cereus | 64.831 |
| WP_098492658 | Bacillus cereus | 64.831 |
| WP_098925877 | Bacillus anthracis | 64.831 |
| WP_101195307 | Bacillus sp. HBCD-sjtu | 64.831 |
| WP_101218479 | Bacillus cereus | 64.831 |
| EEM06723 | Bacillus mycoides Rock1-4 | 64.619 |
| WP_000213623 | Bacillus cereus | 64.619 |
| WP_000213628 | Bacillus thuringiensis | 64.619 |
| WP_000213640 | Bacillus anthracis | 64.619 |
| WP_002114874 | Bacillus cereus | 64.619 |
| WP_002201632 | Bacillus cereus group | 64.619 |
| WP_018767657 | Bacillus | 64.619 |
| WP_033798237 | Bacillus mycoides | 64.619 |
| WP_040119176 | Bacillus mycoides | 64.619 |
| WP_041488274 | Bacillus cereus group | 64.619 |
| WP_042982143 | Bacillus mycoides | 64.619 |
| WP_062821571 | Bacillus cereus | 64.619 |
| WP_070172070 | Bacillus cereus group | 64.619 |
| WP_071771128 | Bacillus sp. NH11B | 64.619 |
| WP_088077715 | Bacillus mycoides | 64.619 |
| WP_088312506 | Bacillus cereus | 64.619 |
| WP_097831246 | Bacillus cereus | 64.619 |
| WP_097926598 | Bacillus toyonensis | 64.619 |
| WP_097988492 | Bacillus pseudomycoides | 64.619 |
| WP_098017785 | Bacillus pseudomycoides | 64.619 |
| WP_098040080 | Bacillus pseudomycoides | 64.619 |
| WP_098101492 | Bacillus pseudomycoides | 64.619 |
| WP_098135232 | Bacillus pseudomycoides | 64.619 |
| WP_098187957 | Bacillus pseudomycoides | 64.619 |
| WP_098226164 | Bacillus toyonensis | 64.619 |
| WP_098362014 | Bacillus cereus | 64.619 |
| WP_098610496 | Bacillus pseudomycoides | 64.619 |
| WP_098639221 | Bacillus anthracis | 64.619 |
| WP_098716171 | Bacillus pseudomycoides | 64.619 |
| WP_101168380 | Bacillus sp. SN10 | 64.619 |
| AIE81030 | Bacillus cereus | 64.482 |
| WP_000213645 | Bacillus cereus | 64.407 |
| WP_002116502 | Bacillus cereus | 64.407 |
| WP_002159137 | Bacillus cereus group | 64.407 |
| WP_006093663 | Bacillus | 64.407 |
| WP_097834495 | Bacillus pseudomycoides | 64.407 |
| WP_097849866 | Bacillus pseudomycoides | 64.407 |
| WP_098160819 | Bacillus pseudomycoides | 64.407 |
| WP_085965931 | Bacillus cereus | 64.271 |
| WP_098814110 | Bacillus pseudomycoides | 64.195 |
| WP_098929011 | Bacillus pseudomycoides | 64.195 |

The disclosure also contemplates the use of variants of Enzyme A in the engineered microorganisms of the disclosure, which include one or more amino acid substitutions, deletions, or additions. Variants can be generated either randomly, or by targeted substitution by using sequence alignments of Enzyme A sequences, such as those described in Table 1. Regions that are conserved and/or important for enzymatic functioning of the Enzyme A can be determined, with variants engineered based on this information. Therefore, Enzyme A can have one or more amino acid substitutions, deletions, or additions which cause the sequence to vary from a native sequence, such as SEQ ID NO: 1.

Optionally, the engineered microorganism includes one or more modifications of genes of the host cell's lower glycolysis pathway. The cell's glycolysis pathway (known as the Embden-Meyerhof-Parnas (EMP) Pathway) has upper and lower portions. In the upper portion glucose (a hexose sugar) is “activated” by phosphorylation with two molecules of adenosine triphosphate (ATP). It is then rearranged in preparation for cleavage into two three carbon (triose) phosphate molecules using fructose bisphosphate aldolase (FBA). In the lower portion, the three carbon (triose) phosphate molecules are phosphorylated and oxidized (via NAD+) in a reaction that is catalyzed by glyceraldehyde dehydrogenase (GAPDH). Subsequently, further phosphorylation provides 4 ATP and 2 pyruvate molecules.

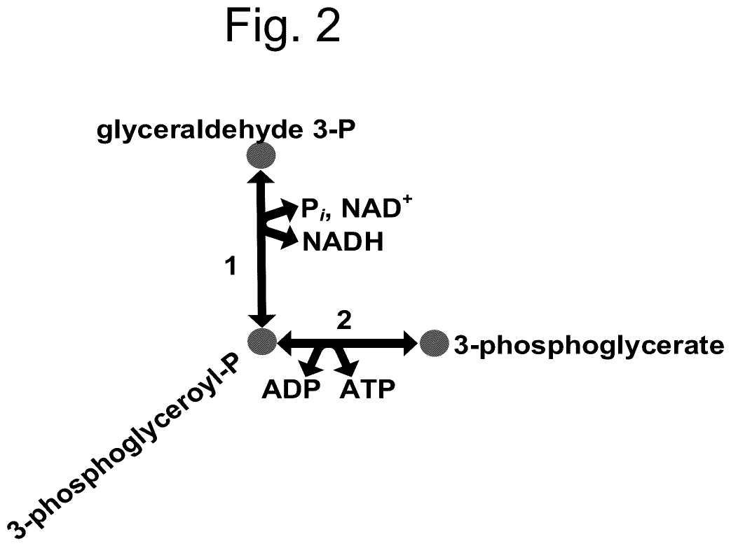

In addition to the introduction of exogenous gapN, the engineered microorganism can include a modification to a native gene of the cell that encodes a NAD-dependent glyceraldehyde-3-phosphate dehydrogenase. See FIG. 2. The modification can attenuate or eliminate the activity of the NAD-dependent glyceraldehyde-3-phosphate dehydrogenase. An example of such a gene that can be modified or deleted to attenuate or eliminate activity is the E. coli gapA gene (encodes glyceraldehyde-3-phosphate dehydrogenase A; P0A9B2) which uses the cofactor NAD and phosphate to catalyze the oxidative phosphorylation of G3P to 13BPG. An engineered E. coli wherein gapA is deleted or otherwise its expression reduced can be referred to as ΔgapA, or any modification which reduces enzymatic activity may be notated as such.

The deletion, reduced expression, or reduced enzymatic activity of GapA, can allow the exogenous GapN to more effectively compete for glyceraldehyde-3-phosphate (G3P) as a substrate. Since GapN displays slower conversion of G3P to 3-PG as compared to E. coli GapA conversion of G3P to 1,3-BPG, this can provide a way to enhance the pool size of the one or more phosphorylated 3 and/or 4 carbon species. In turn, this improves the balance between regeneration of C5 compounds and lower glycolysis, and enables or enhances synthetic methylotrophy in the cells having these modifications.

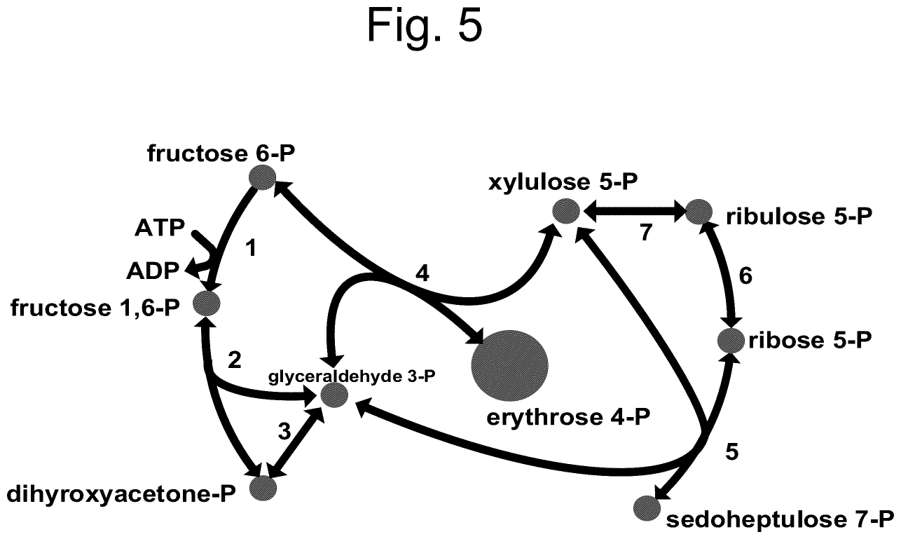

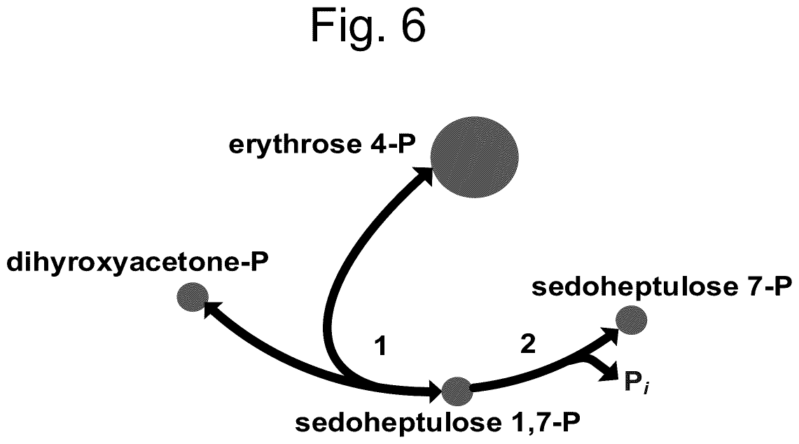

In one aspect of the disclosure synthetic methylotrophy is promoted by engineering the cell to provide a desired balance between lower glycolysis through glyceraldehyde-3-phosphate dehydrogenase (GapA or GapN) and C5 regeneration through the RuMP cycle (Fba, GlpX). See FIGS. 5 and 6. The genetic modifications set forth in the disclosure that provide the desired balance can be made in native E. coli, which is regulated by NADH levels, and take into account the use of exogenous methanol dehydrogenase which generates NADH (and which otherwise presents additional challenges to attain this balance). In embodiments, the use of GapN therefore not only facilitates synthetic methylotrophy by its kinetic activity, but also by its unique property to reduce NADP to NADPH. One or more genes that are part of the lower glycolysis pathway, and that are “downstream” of GapA, can also be modified to attenuate or eliminate their activity. For example, the engineered microorganism can have one or more modification(s) that (a) attenuates or eliminates an enzyme in a pathway leading from glyceraldehyde-3-phosphate to phosphoenolpyruvate (PEP).

With reference to FIG. 2, in some embodiments, the engineered microorganism can have one or more modification(s) that (a) attenuates or eliminates a phosphoglycerate kinase activity (e.g., E. coli pgk, Uniprot P0A799). Pgk converts 3-phospho-D-glyceroyl phosphate to 3-phospho-D-glycerate, while generating an ATP from ADP via substrate level phosphorylation. In some embodiments, the engineered microorganism can have one or more modification(s) that (a) attenuates or eliminates a phosphoglycerate mutase activity.

In aspects, engineered cells of the disclosure also include exogenous enzyme B which is (bi) a hexulose-6-phosphate synthase, (bii) a 6-phospho-3-hexuloisomerase, (biii) a phosphoketolase, or any combination of (bi), (bii) and (biii).

In methanotrophic bacteria, formaldehyde made from methane and methanol oxidation is used to form metabolic intermediates in pathways leading to the formation of cellular products (Anthony, C. (1991) Biotechnology 18:79-109). The serine and D-ribulose 5-phosphate (RuMP) pathways use formaldehyde to produce carbon-containing intermediate compounds which are subsequently converted into other downstream products.

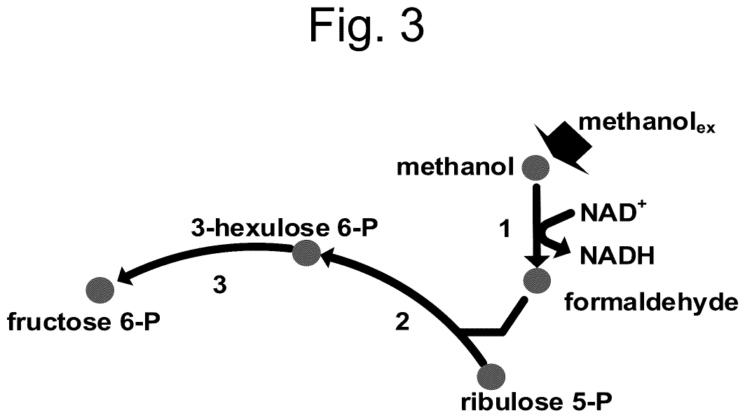

With reference to FIG. 3, the RuMP pathway hexulose-6-phosphate synthase (HPS) enzymatically condenses formaldehyde and D-ribulose 5-phosphate (RuMP) to form hexulose 6-phosphate (HuMP). 6-phospho-3-hexuloisomerase (PHI) enzymatically converts HuMP to β-D-fructofuranose 6-phosphate (F6P). HPS and PHI are unique to natural organisms that have the RuMP pathway. For every one molecule of formaldehyde assimilated, one molecule of F6P is created. F6P can then be cleaved to 3-carbon compounds by either of two routes. Enzymes of these other routes are not exclusive to those methanotrophic bacteria expressing HPS and HPI

In one route 6-phosphofructokinase (EC 2.7.1.11) phosphorylates F6P to fructose 1,6-bisphosphate (FDP). Fructose-bisphosphate aldolase (EC 4.1.2.13) then cleaves FDP into dihydroxy acetone phosphate (DHAP) and glyceraldehyde 3-phosphate (G3P).

In another route glucose-6-phosphate isomerase (EC 5.3.1.9) isomerizes FMP to glucose 6-phosphate (G6P). Glucose-6-phosphate 1-dehydrogenase (EC 1.1.1.49) then dehydrogenates G6P to D-glucono-1,5-lactone 6-phosphate which is further dehydrogenated to 6-phospho-gluconate by 6-phosphogluconolactonase (EC 3.1.1.31). Phosphogluconate dehydratase (EC 4.2.1.12) then converts 6-phospho-gluconate to 2-keto-3-deoxy-6-phospho-D-gluconate (KDPG). Subsequently, KDPG aldolase (EC 4.1.2.14) cleaves KDPG into glyceraldehyde 3-phosphate (G3P) and pyruvate. Pyruvate and DHAP formed through this pathway can be used in cellular pathways for the synthesis of biomolecules.

3-hexulose-6-phosphate synthases are of the enzyme class (EC) 4.1.2.43. The enzyme 3-hexulose-6-phosphate synthase (HPS) can carry out the fixation of formaldehyde with ribulose 5-phosphate (Ru5P) to form d-arabino-3-hexulose-6-phosphate (Hu6P). See FIG. 2.

One exemplary HPS sequence is based on Bacillus methanolicus MGA HPS (Genbank Accession number AAR39392.1; 211 amino acids long; SEQ ID NO: 2). The engineered microorganism can express a HPS sequence that is related to Bacillus methanolicus MGA HPS, such as a Bacillus methanolicus MGA HPS homolog. For example, the HPS sequence can have 25% or greater, 30% or greater, 40% or greater, 50% or greater, 60% or greater, 70% or greater, 80% or greater, 85% or greater, 90% or greater, or 95% or greater identity to SEQ ID NO: 2. Homologs of SEQ ID NO: 2 can be identified by sequence identity searching (e.g., algorithms well known to those skilled in the art, such as Align, BLAST, Clustal W, as described herein).

Exemplary HPS sequences include those found in Table 2A and 2B below. For example, 3-hexulose-6-phosphate synthase include, but are not limited to, Bacillus methanolicus PB1 HPS (EU81375.1); Methylobacillus flagellates HPS (YP 544362.1); Methylobacillus flagellatus HPS (YP 544363.1); Bacillus subtilis HPS (NP_388228.1); Methylophilus methylotrophus HPS (WP_018986666.1); Methylophilus methylotrophus ATCC 53528 HPS (WP_018985298.1); Aminomonas aminovorus HPS (AAG29505.1), Amycolatopsis methanolica 239 HPS (AJ24611.1); Geobacillus sp. GHH01 HPS (YP_007402409.1); Geobacillus sp. M10EXG HPS (AAR91478.1); Geobacillus sp. Y4.1MC1 HPS (YP_003990382.1); Geobacillus thermodenitrificans NG80-2 HPS (WP_008879217.1); Methylomonas aminofaciens HPS (BAA83096.1); Methylovorus glucosetrophus SIP3-4 HPS (YP_003050044.1); Methylovorus sp. MP688 HPS (YP_004038706.1); and Mycobacterium gastri HPS (BAA90546.1).

TABLE 2A
Pairwise alignment (% ID)

| | Geobacillus | | | | | | |
|---|---|---|---|---|---|---|---|
| | thermo- | Bacillus | Bacillus | | | | |
| | denitrificans | methanolicus | methanolicus | Geobacillus | Geobacillus | Geobacillus | Methylophilus |
| HPS | NG80-2 | MGA3 | PB1 | sp. GHH01 | sp. M10EXG | sp. Y4.1MC1 | methylotrophus |
| Geobacillus | 100 | 30.8 | 30.8 | 30.3 | 29.9 | 29.9 | 31.8 |
| thermo- | | | | | | | |
| denitrificans | | | | | | | |
| NG80-2 | | | | | | | |
| Bacillus | 30.8 | 100 | [symbol] 8.1 | 76.8 | 76.8 | 77.3 | 38.4 |
| methanolicus | | | | | | | |
| MGA3 | | | | | | | |
| Bacillus | 30.8 | 98.1 | 100 | 76.3 | 76.3 | 76.8 | 37.9 |
| methanolicus | | | | | | | |
| PB1 | | | | | | | |
| Geobacillus | 30.3 | 76.8 | 76.3 | 100 | 97.6 | 98.1 | 38.9 |
| sp. GHH01 | | | | | | | |
| Geobacillus | 29.9 | 76.8 | 76.3 | 97.6 | 100 | [symbol] .5 | 38.4 |
| sp. M10EXG | | | | | | | |
| Geobacillus | 29.9 | 77.3 | 76.8 | [symbol] 8.1 | 99.5 | 100 | 38.4 |
| sp. Y4.1MC1 | | | | | | | |
| Methylophilus | 31.8 | 38.4 | 37.9 | 38.9 | 38.4 | 38.4 | 100 |
| methylotrophus | | | | | | | |
| Methylophilus | 32.1 | 34.4 | 34 | 38.3 | 37.8 | 37.8 | 81.9 |
| methylotrophus | | | | | | | |
| ATCC 53528 | | | | | | | |
| Methylo- | 33.2 | 37.5 | 37 | 40.9 | 40.4 | 40.4 | 83.7 |
| bacillus | | | | | | | |
| flagellatus | | | | | | | |
| Methylomonas | 32.7 | 36.5 | 36.1 | 39.9 | 3 [symbol] .4 | 3 [symbol] .4 | 84.7 |
| aminofaciens | | | | | | | |
| Methylovorus | 31.5 | 37.2 | 36.7 | 38.2 | 37.7 | 37.7 | 80.4 |
| glucosetrophus | | | | | | | |
| SIP3-4 | | | | | | | |
| Aminomonas | 31.3 | 37 | 36.5 | 37.9 | 37.4 | 37.4 | 76.1 |
| aminovorus | | | | | | | |
| Methylo- | 31.3 | 37 | 36.5 | 37.9 | 37.4 | 37.4 | 76.5 |
| bacillus | | | | | | | |
| flagellatus | | | | | | | |
| Amycolatopsis | 33.8 | 40 | 38.6 | 37.6 | 38.1 | 38.1 | 46.2 |
| methanolica | | | | | | | |
| 239 | | | | | | | |
| Mycobacterium | 32.7 | 39.4 | 39.9 | 39.4 | 38.9 | 38.9 | 46.6 |
| gastri | | | | | | | |

[symbol]

indicates data missing or illegible when filed

TABLE 2B
Pairwise alignment(% ID)

| | Methylophilus | Methylo- | | Methylovorus | | Methylo- | Amycolatopsis | |
|---|---|---|---|---|---|---|---|---|
| | methylotrophus | bacillus | Methylomonas | glucosetrophus | Aminomonas | bacillus | methanolica | Mycobacterium |
| HPS | ATCC 53528 | flagellatus | aminofaciens | SIP3-4 | aminovorus | flagellatus | 239 | gastri |
| Geobacillus | 32.1 | 33.2 | 32.7 | 31.5 | 31.3 | 31.3 | 3 [symbol] .8 | 32.7 |
| thermo- | | | | | | | | |
| denitrificans | | | | | | | | |
| NG80-2 | | | | | | | | |
| Bacillus | 34.4 | 37.5 | 36.5 | 37.2 | 37 | 37 | 40 | 39.4 |
| methanolicus | | | | | | | | |
| MGA3 | | | | | | | | |
| Bacillus | 34 | 37 | 36.1 | 36.7 | 36.5 | 36.5 | 3 [symbol] . [symbol] | 3 [symbol] .9 |
| methanolicus | | | | | | | | |
| PB1 | | | | | | | | |
| Geobacillus | 38.3 | 40.9 | 38.9 | 38.2 | 37.9 | 37.9 | 37.6 | 39.4 |
| sp. GHH01 | | | | | | | | |
| Geobacillus | 37.8 | 40.4 | 39.4 | 37.7 | 37.4 | 37.4 | 38.1 | 38.9 |
| sp. M10EXG | | | | | | | | |
| Geobacillus | 37.8 | 40.4 | 39.4 | 37.7 | 37.4 | 37.4 | 38.1 | 38.9 |
| sp. | | | | | | | | |
| Y4.1MC1 | | | | | | | | |
| Methylophilus | 81.9 | 83.7 | 84.7 | 30.4 | 7 [symbol] .1 | 76.5 | 46.2 | 46.6 |
| methylotrophus | | | | | | | | |
| Methylophilus | 100 | 87.1 | 87.6 | 82 | 8 [symbol] .3 | [symbol] .8 | 47.1 | 47.1 |
| methylotrophus | | | | | | | | |
| ATCC 53528 | | | | | | | | |
| Methylo- | 87.1 | 100 | 97.1 | 38.2 | 87.6 | 86.6 | 47.4 | 47.1 |
| bacillus | | | | | | | | |
| flagellatus | | | | | | | | |
| Methylomonas | 87.6 | 97.1 | 100 | [symbol] .2 | 86.1 | 86.1 | 46.9 | 45.7 |
| aminofaciens | | | | | | | | |
| Methylovorus | 82 | 88.2 | 89.2 | 100 | 90.5 | 90.5 | 45.5 | 4 [symbol] .6 |
| glucosetrophus | | | | | | | | |
| SIP3-4 | | | | | | | | |
| Aminomonas | 83.3 | 87.6 | 86.1 | 90.5 | 100 | 97.4 | 47.6 | 47.1 |
| aminovorus | | | | | | | | |
| Methylo- | 83.8 | 86.6 | [symbol] .1 | [symbol] 0.5 | 97.4 | 100 | 47.5 | 46.2 |
| bacillus | | | | | | | | |
| flagellatus | | | | | | | | |
| Amycolatopsis | 47.1 | 47.4 | 46.9 | 46.6 | 47.6 | 47.6 | 100 | 60.1 |
| methanolica | | | | | | | | |
| 239 | | | | | | | | |
| Mycobacterium | 47.1 | 47.1 | 45.7 | 45.6 | 47.1 | 46.2 | 60.1 | 100 |
| gastri | | | | | | | | |

[symbol]

indicates data missing or illegible when filed

The disclosure also contemplates the use of variants of HPS enzymes in the engineered microorganisms of the disclosure, which include one or more amino acid substitutions, deletions, or additions. Variants can be generated either randomly, or by targeted substitution by using sequence alignments of HPS sequences, such as those described in Tables 2A and 2B. Regions that are conserved and/or important for enzymatic functioning of the HPS enzymes can be determined, with variants engineered based on this information. Therefore, the HPS enzymes can have one or more amino acid substitutions, deletions, or additions which cause the sequence to vary from a native sequence, such as SEQ ID NO: 2.

6-phospho-3-hexuloisomerases are of the enzyme class (EC) 5.3.1.27. 6-phospho-3-hexuloisomerases are of the enzyme class (EC) 5.3.1.27. 6-phospho-3-hexuloisomerase activity (PHI), can carry out the isomerization of d-arabino-3-hexulose-6-phosphate (Hu6P) to fructose 6-phosphate (F6P). One exemplary PHI sequence is based on Bacillus methanolicus MGA3 PHI (Genbank Accession number AAR39393.1, 184 amino acids long; SEQ ID NO: 3). The engineered microorganism can express a PHI sequence that is related to Bacillus methanolicus MGA3 PHI, such as a Bacillus methanolicus MGA PHI homolog. For example, the PHI sequence can be 30% or greater, 40% or greater, 50% or greater, 60% or greater, 70% or greater, 80% or greater, 85% or greater, 90% or greater, or 95% or greater identity to SEQ ID NO: 3. Homologs of SEQ ID NO: 3 can be identified by sequence identity searching (e.g., algorithms well known to those skilled in the art, such as Align, BLAST, Clustal W, as described herein).

Exemplary PHI sequences include those found in Table 3A and 3B below. For example, 6-phospho-3-hexuloisomerase sequences include, but are not limited to Bacillus methanolicus PB1 PHI (EU81376.1); Mycobacterium gastri PB1 PHI (BAA90545.1); Methylobacillus flagellatus KT PHI (YP 545762.1); Bacillus subtilis PHI (NP_388227.1); Methylophilus methylotrophus ATCC 53528 HPS (WP_018985297.1); Amycolatopsis methanolica 239 PHI (AU24609.1); Geobacillus sp. GHH01 PHI (YP_007402408.1); Geobacillus sp. Y4.1MC1 PHI (YP_003990383.1); Geobacillus thermodenitrificans NG80-2 PHI (WP_011887353.1); Methylomonas aminofaciens PHI (BAA83098.1); Methylovorus glucosetrophus SIP3-4 PHI (YP_003051269.1); and Methylovorus sp. MP688 PHI (ADQ84715.1).

TABLE 3A
Pairwise alignment (% ID)

| | | | Methylophilus | Methylovorus | Geobacillus |
|---|---|---|---|---|---|
| | Methylobacillus | Methylomonas | methylotrophus | glucosetrophus | thermodenitrificans |
| PHI | flagellatus KT | aminofaciens | ATCC 53528 | SIP3-4 | NG80-2 |
| Methylobacillus | 100 | 95.6 | 58.9 | 64.4 | 31.1 |
| flagellatus KT | | | | | |
| Methylomonas | 95.6 | 100 | 59.4 | 65 | 30.6 |
| aminofaciens | | | | | |
| Methylophilus | 58.9 | 59.4 | 100 | 86.5 | 28 |
| methylotrophus | | | | | |
| ATCC 53528 | | | | | |
| Methylovorus | 64.4 | [symbol] | 86.5 | 100 | 29.7 |
| glucosetrophus | | | | | |
| SIP3-4 | | | | | |
| Geobacillus | 31.1 | 30.6 | 28 | 29.7 | 100 |
| thermodenitrificans | | | | | |
| NG80-2 | | | | | |
| Bacillus | 32.4 | 33 | 32 | 33.1 | 37.2 |
| methanolicus MGA3 | | | | | |
| Bacillus | 32.4 | 33 | 32 | 33.1 | 37.2 |
| methanolicus PB1 | | | | | |
| Geobacillus sp. | 35 | 34.4 | 32.4 | 32.4 | 41.5 |
| GHH01 | | | | | |
| Geobacillus sp. | 3 [symbol] .9 | 34.4 | 33 | 33.5 | 39.9 |
| Y4.1MC1 | | | | | |
| Amycolatopsis | 31.3 | 31.9 | 27.1 | 27.6 | 36.6 |
| methanolica 239 | | | | | |
| Mycobacterium | 33 | [symbol] 3 | 31.5 | [symbol] .1 | 37.7 |
| gastri | | | | | |

[symbol]

indicates data missing or illegible when filed

TABLE 3B
Pairwise alignment(% ID)

| | Bacillus | Bacillus | | | Amycolatopsis | |
|---|---|---|---|---|---|---|
| | methanolicus | methanolicus | Geobacillus | Geobacillus | methanolica | Mycobacterium |
| PHI | MGA3 | PB1 | sp. GHH01 | sp. Y4.1MC1 | 239 | gastri |
| Methylobacillus | 32.4 | 32.4 | 35 | 33.9 | 31.3 | 33 |
| flagellatus KT | | | | | | |
| Methylomonas | 33 | 33 | 34.4 | 34.4 | 31.9 | 33 |
| aminofaciens | | | | | | |
| Methylophilus | 32 | 32 | 32.4 | 33 | 27.1 | 31.5 |
| methylotrophus | | | | | | |
| ATCC 53528 | | | | | | |
| Methylovorus | 33.1 | 33.1 | 32.4 | 33.5 | 27.6 | 33.1 |
| glucosetrophus | | | | | | |
| SIP3-4 | | | | | | |
| Geobacillus | 37.2 | 37.2 | 41.5 | 39.9 | 36.6 | 37.7 |
| thermo- | | | | | | |
| denitrificans | | | | | | |
| NG80-2 | | | | | | |
| Bacillus | 100 | 98.9 | 75.1 | 76.8 | 32.6 | 37.5 |
| methanolicus MGA3 | | | | | | |
| Bacillus | 98.9 | 100 | 74.6 | 76.2 | 32.6 | 38.6 |
| methanolicus PB1 | | | | | | |
| Geobacillus | 75.1 | 74.6 | 100 | 33 | 33 | 36.2 |
| sp. GHH01 | | | | | | |
| Geobacillus | 76.8 | 76.2 | 99 | 100 | 32.6 | 38 |
| sp. Y4.1MC1 | | | | | | |
| Amycolatopsis | 32.6 | 32.6 | 33 | 32.6 | 100 | 47.7 |
| methanolica 239 | | | | | | |
| Mycobacterium | 37. [symbol] | 38.6 | 36.2 | 38 | 47.7 | 100 |
| gastri | | | | | | |

[symbol]

indicates data missing or illegible when filed

The disclosure also contemplates the use of variants of PHI enzymes in the engineered microorganisms of the disclosure, which include one or more amino acid substitutions, deletions, or additions. Variants can be generated either randomly, or by targeted substitution by using sequence alignments of PHI sequences, such as those described in Tables 3A and 3B. Regions that are conserved and/or important for enzymatic functioning of the PHI enzymes can be determined, with variants engineered based on this information. Therefore, the PHI enzymes 10 can have one or more amino acid substitutions, deletions, or additions which cause the sequence to vary from a native sequence, such as SEQ ID NO: 3.

Engineered cells of the disclosure can also express HPS-PHI fusion protein, or variants thereof. The engineered fusion protein can include a polypeptide sequence based on natural HPS-PHI fusions, such as, a fusion from Methylococcus capsulatas (YP_115138.1); Methylomicrobium album BG8 (EIC30826.1); Pyrococcus abyssi (NP_127388.1); Pyrococcus furiosus (NP_577949.1); Pyrococcus horikoshii OT3 (NP_143767.1), or Thermococcus kodakaraensis (YP_182888.1). Exemplary fusion proteins including HPS-PHI sequences are described in WO2017/075208 (Barton et at) the disclosure of which is incorporated herein by reference.

The engineered microorganism can include an exogenous enzyme (“enzyme B”) that is a phosphoketolase, such as fructose-6-phosphate phosphoketolase or xylulose-5-phosphate phosphoketolase.

Conversion of fructose-6-phosphate and phosphate to acetyl-phosphate and erythrose-4-phosphate (E4P) can be carried out by fructose-6-phosphate phosphoketolase (EC 4.1.2.22) Conversion of fructose-6-phosphate and phosphate to acetyl-phosphate and erythrose-4-phosphate is one of the key reactions in the Bifidobacterium shunt. There is evidence for the existence of two distinct phosphoketolase enzymes in bifidobacteria (Sgorbati et al, 1976, Antonie Van Leeuwenhoek, 42(1-2) 49-57; Grill et al, 1995, Curr Microbiol, 31(1);49-54). The enzyme from Bifidobacterium dentium appeared to be specific solely for fructose-6-phosphate (EC: 4.1.2.22) while the enzyme from Bifidobacterium pseudolongum subsp. globosum is able to utilize both fructose-6-phosphate and D-xylulose 5-phosphate (EC: 4.1.2.9) (Sgorbati et al, 1976, Antonie Van Leeuwenhoek, 42(1-2) 49-57). The enzyme encoded by the xfp gene, originally discovered in Bifidobacterium animalis lactis, is the dual-specificity enzyme (Meile et al., 2001, J Bacteriol, 183, 2929-2936; Yin et al, 2005, FEMS Microbiol Lett, 246(2); 251-257). Additional phosphoketolase enzymes can be found in Leuconostoc mesenteroides (Le et al, Biotechnol Lett. 2005 June; 27(12):853-8), Clostridium acetobutylicum ATCC 824 (Servinsky et al, Journal of Industrial Microbiology & Biotechnology, 2012, 39, 1859-1867), Aspergillus nidulans (Kocharin et al, 2013, Biotechnol Bioeng, 110(8), 2216-2224; Papini, 2012, Appl Microbiol Biotechnol, 95 (4), 1001-1010), Bifidobacterium breve (Suziki et al, 2010, Acta Crystallogr Sect F Struct Biol Cryst Commun., 66 (Pt 8):941-3), Lactobacillus paraplantarum (Jeong et al, 2007, J Microbiol Biotechnol, 17(5), 822-9).

Conversion of xylulose-5-phosphate and phosphate to acetyl-phosphate and glyceraldehyde-3-phosphate can be carried out by xylulose-5-phosphate phosphoketolase (EC 4.1.2.9). There is evidence for the existence of two distinct phosphoketolase enzymes in bifidobacteria (Sgorbati et al, 1976, Antonie Van Leeuwenhoek, 42(1-2) 49-57; Grill et al, 1995, Curr Microbiol, 31(1); 49-54). The enzyme from Bifidobacterium dentium appeared to be specific solely for fructose-6-phosphate (EC: 4.1.2.22) while the enzyme from Bifidobacterium pseudolongum subsp. globosum is able to utilize both fructose-6-phosphate and D-xylulose 5-phosphate (EC: 4.1.2.9) (Sgorbati et al, 1976, Antonie Van Leeuwenhoek, 42(1-2) 49-57). Many characterized enzymes have dual-specificity for xylulose-5-phosphate and fructose-6-phosphate. The enzyme encoded by the xfp gene, originally discovered in Bifidobacterium animalis lactis, is the dual-specificity enzyme (Meile et al., 2001, J Bacteriol, 183, 2929-2936; Yin et al, 2005, FEMS Microbiol Lett, 246(2); 251-257). Additional phosphoketolase enzymes can be found in Leuconostoc mesenteroides (Lee et al, Biotechnol Lett. 2005 June; 27(12):853-8), Clostridium acetobutylicum ATCC 824 (Servinsky et al, Journal of Industrial Microbiology & Biotechnology, 2012, 39, 1859-1867), Aspergillus nidulans (Kocharin et al, 2013, Biotechnol Bioeng, 110(8), 2216-2224; Papini, 2012, Appl Microbiol Biotechnol, 95 (4), 1001-1010), Bifidobacterium breve (Suziki et al, 2010, Acta Crystallogr Sect F Struct Biol Cryst Commun., 66 (Pt 8):941-3), and Lactobacillus paraplantarum (Jeong et al, 2007, J Microbiol Biotechnol, 17(5), 822-9).

Exemplary phosphoketolase enzymes include those found in Table 4 below.

TABLE 4

| Protein | GENBANK ID | GI NUMBER | ORGANISM |
|---|---|---|---|
| Xfp | YP_006280131.1 | 386867137 | Bifidobacterium |
| | | | animalis lactis |
| Xfp | AAV66077.1 | 55818565 | Leuconostoc |
| | | | mesenteroides |
| CAC1343 | NP_347971.1 | 15894622 | Clostridium |
| | | | acetobutylicum |
| | | | ATCC 824 |
| XpkA | CBF76492.1 | 259482219 | Aspergillus nidulans |
| Xfp | AAR98788.1 | 41056827 | Bifidobacterium |
| | | | pseudolongum subsp. |
| | | | globosum |
| Xfp | WP_022857642.1 | 551237197 | Bifidobacterium |
| | | | pseudolongum subsp. |
| | | | globosum |
| Xfp | ADF97524.1 | 295314695 | Bifidobacterium breve |
| Xfp | AAQ64626.1 | 34333987 | Lactobacillus |
| | | | paraplantarum |

In some embodiments, the engineered organism includes a fructose-6-phosphate phosphoketolase and optionally a phosphotransacetylase. In some embodiments, the engineered organism includes a fructose-6-phosphate phosphoketolase and optionally an acetyl-CoA transferase, an acetyl-CoA synthetase, or an acetyl-CoA ligase.

The formation of acetyl-CoA from acetyl-phosphate can be catalyzed by phosphotransacetylase (EC 2.3.1.8). The pta gene from E. coli encodes an enzyme that reversibly converts acetyl-CoA into acetyl-phosphate (Suzuki, T., Biochim. Biophys. Acta 191:559-569 (969)). Additional acetyltransferase enzymes have been characterized in Bacillus subtilis (Rado and Hoch, Biochim. Biophys. Acta 321:114-125 (1973), Clostridium kluyveri (Stadtman, E., Methods Enzymol. 1:5896-599 (1955), and Thermotoga maritima (Bock et. al.., J. Bacteriol. 181:1861-1867 (1999)). This reaction can also be catalyzed by some phosphotransbutyrylase enzymes (EC 2.3.1.19), including the ptb gene products from Clostridium acetobutylicum (Wiesenborn et al., App. Environ. Microbiol. 55:317-322 (1989); Walter et al., Gene 134:107-111 (1993)). Additional ptb genes are found in butyrate-producing bacterium L2-50 (Louis et al., J. Bacteriol. 186:2099-2106 (2004) and Bacillus megaterium (Vazquez et al., Curr. Microbiol. 42:345-349 (2001). Homologs to the E. coli pta gene exist in several other organisms including Salmonella enterica and Chlamydomonas reinhardtii.

Exemplary phosphotransacetylase enzymes include those found in Table 5 below.

TABLE 5

| Protein | GenBank ID | GI Number | Organism |
|---|---|---|---|
| Pta | NP_416800.1 | 71152910 | Escherichia coli |
| Pta | P39646 | 730415 | Bacillus subtilis |
| Pta | A5N801 | 146346896 | Clostridium kluyveri |
| Pta | Q9X0L4 | 6685776 | Thermotoga maritime |
| Ptb | NP_349676 | 34540484 | Clostridium acetobutylicum |
| Ptb | AAR19757.1 | 38425288 | butyrate-producing bacterium |
| | | | L2-50 |
| Ptb | CAC07932.1 | 10046659 | Bacillus megaterium |
| Pta | NP_461280.1 | 16765665 | Salmonella enterica subsp. |
| | | | enterica serovar Typhimurium |
| | | | str. LT2 |
| PAT2 | XP_001694504.1 | 159472743 | Chlamydomonas reinhardtii |
| PAT1 | XP_001691787.1 | 159467202 | Chlamydomonas reinhardtii |

The acylation of acetate to acetyl-CoA can be catalyzed by enzymes with acetyl-CoA synthetase, ligase or transferase activity. Two enzymes that can catalyze this reaction are AMP-forming acetyl-CoA synthetase or ligase (EC 6.2.1.1) and ADP-forming acetyl-CoA synthetase (EC 6.2.1.13). AMP-forming acetyl-CoA synthetase (ACS) is the predominant enzyme for activation of acetate to acetyl-CoA. Exemplary ACS enzymes are found in E. coli (Brown et al., J. Gen. Microbiol. 102:327-336 (1977)), Ralstonia eutropha (Priefert and Steinbuchel, J. Bacteriol. 174:6590-6599 (1992)), Methanothermobacter thermautotrophicus (Ingram-Smith and Smith, Archaea 2:95-107 (2007)), Salmonella enterica (Gulick et al., Biochemistry 42:2866-2873 (2003)) and Saccharomyces cerevisiae (Jogl and Tong, Biochemistry 43:1425-1431 (2004)). ADP-forming acetyl-CoA synthetases are reversible enzymes with a generally broad substrate range (Musfeldt and Schonheit, J. Bacteriol. 184:636-644 (2002)). Two isozymes of ADP-forming acetyl-CoA synthetases are encoded in the Archaeoglobus fulgidus genome by are encoded by AF1211 and AF1983 (Musfeldt and Schonheit, supra (2002)). The enzyme from Haloarcula marismortui (annotated as a succinyl-CoA synthetase) also accepts acetate as a substrate and reversibility of the enzyme was demonstrated (Brasen and Schonheit, Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetate, isobutyryl-CoA (preferred substrate) and phenylacetyl-CoA (Brasen and Schonheit, supra (2004)). Directed evolution or engineering can be used to modify this enzyme to operate at the physiological temperature of the host organism. The enzymes from A.fulgidus, H. marismortui and P. aerophilum have all been cloned, functionally expressed, and characterized in E. coli (Brasen and Schonheit, supra (2004); Musfeldt and Schonheit, supra (2002)). Additional candidates include the succinyl-CoA synthetase encoded by sucCD in E. coli (Buck et al., Biochemistry 24:6245-6252 (1985)) and the acyl-CoA ligase from Pseudomonas putida (Fernandez-Valverde et al., Appl. Environ. Microbiol. 59:1149-1154 (1993)). The aforementioned proteins are shown in Table 6 below.

TABLE 6

| Protein | GenBank ID | GI Number | Organism |
|---|---|---|---|
| Acs | AAC77039.1 | 1790505 | Escherichia coli |
| AcoE | AAA21945.1 | 141890 | Ralstonia eutropha |
| Acs1 | ABC87079.1 | 86169671 | Methanothermobacter |
| | | | thermautotrophicus |
| Acs1 | AAL23099.1 | 16422835 | Salmonella enterica |
| ACS1 | Q01574.2 | 257050994 | Saccharomyces cerevisiae |
| AF1211 | NP_070039.1 | 11498810 | Archaeoglobus fulgidus |
| AF1983 | NP_070807.1 | 11499565 | Archaeoglobus fulgidus |
| Scs | YP_135572.1 | 55377722 | Haloarcula marismortui |
| PAE3250 | NP_560604.1 | 18313937 | Pyrobaculum aerophilum |
| | | | str. IM2 |
| SucC | NP_415256.1 | 16128703 | Escherichia coli |
| SucD | AAC73823.1 | 1786949 | Escherichia coli |
| PaaF | AAC24333.2 | 22711873 | Pseudomonas putida |

In some embodiments, the engineered organism includes an exogenous alcohol dehydrogenase, such as an exogenous methanol dehydrogenase.

Alcohol dehydrogenases (ADHs; EC 1.1.1.1) promote the conversion of alcohols to and aldehydes or ketones, typically along with the reduction of nicotinamide adenine dinucleotide (NAD⁺ to NADH). ADHs are instrumental in the generation of important compounds having aldehyde, ketone, and alcohol groups during biosynthesis of various metabolites.

One class of alcohol dehydrogenase is methanol dehydrogenases (MDHs). MDHs, converts methanol (MeOH) to formaldehyde (Fald), may be used in an enzymatic pathway engineered into microorganisms of the disclosure to enable MeOH as a sole carbon source or as a co-carbon source with other feed stocks. See FIG. 3. Engineered cells of the disclosure can include a natural or a non-natural NAD⁺-dependent methanol dehydrogenases (MDHs), in particular enzymes of the class EC 1.1.1.244.

One exemplary MDH sequence is an NAD(P)+-dependent methanol dehydrogenase from Bacillus methanolicus MGA3 (Genbank Accession number EU77596.1, GI number: 387585261; designated herein as MDH 2315, 382 amino acids long; SEQ ID NO: 4). MDH 2315 is reported in the literature as an NAD(P)-dependent methanol dehydrogenase from Bacillus methanolicus MGA3 and its sequence was described in Brautaset et al., “Plasmid-Dependent Methylotrophy in Thermotolerant Bacillus methnolicus”, Journal of Bacteriology, vol. 186, pp 1229-1238 (2004). It is also referred to as MDH MGA3 in WO2013/110797 to Brautaset and MDH “M” in Krog et al., “Methylotrophic Bacillus methanolicus Encodes Two Chromosomal and One Plasmid Born NAD+ Dependent Methanol Dehydrogenase Paralogs with Different Catalytic and Biochemical Properties”, PLOS ONE, pp. 1-11, (2013), which report additional wild-type Bacillus MDHs.

The engineered microorganism can express a MDH sequence that is related to Bacillus methanolicus MGA3 MDH, such as a Bacillus methanolicus MGA MDH homolog. For example, the PHI sequence can be 20% or greater, 30% or greater, 40% or greater, 50% or greater, 60% or greater, 70% or greater, 80% or greater, or 90% or greater identity to SEQ ID NO: 4. Homologs of SEQ ID NO: 4 can be identified by sequence identity searching (e.g., algorithms well known to those skilled in the art, such as Align, BLAST, Clustal W, as described herein).

Exemplary MDH enzymes include those found in Table 7 below.

TABLE 7

| | | | AA | % Identity |
|---|---|---|---|---|
| GenBankID | GI No. | Organism | length | (global) |
| EIJ77596.1 | 387585261 | Bacillus methanolicus MGA3 | 382 | 100 |
| AAA22593.1 | 143175 | Bacillus methanolicus C1 | 381 | 97 |
| EIJ77618.1 | 387585284 | Bacillus methanolicus PB1 | 383 | 93 |
| EIJ78790.1 | 387586466 | Bacillus methanolicus PB1 | 383 | 90 |
| EIJ80770.1 | 387588449 | Bacillus methanolicus MGA3 | 385 | 62 |
| EIJ78397.1 | 387586073 | Bacillus methanolicus PB1 | 385 | 61 |
| EIJ83020.1 | 387590701 | Bacillus methanolicus MGA3 | 385 | 61 |
| EFI69743.1 | 298729190 | Lysinibacillus fusiformis | 401 | 56 |
| YP_004860127.1 | 347752562 | Bacillus coagulans 36D1 | 386 | 56 |
| YP_001699778.1 | 169829620 | Lysinibacillus sphaericus | 402 | 54 |
| ZP_11313277.1 | 410459529 | Bacillus azotoformans LMG | 386 | 54 |
| | | 9581 | | |
| ZP_05587334.1 | 257139072 | Burkholderia thailandensis | 390 | 54 |
| | | E264 | | |
| YP_004681552.1 | 339322658 | Cupriavidus necator N-1 | 390 | 53 |
| AGF87161 | 451936849 | uncultured organism | 393 | 53 |
| YP_002138168.1 | 197117741 | Geobacter bemidjiensis Bem | 387 | 52 |
| YP_359772.1 | 78043360 | Carboxydothermus | 383 | 52 |
| | | hydrogenoformans Z-2901 | | |
| YP_001343716.1 | 152978087 | Actinobacillus succinogenes | 385 | 51 |
| | | 130Z | | |
| ZP_16224338.1 | 421788018 | Acinetobacter baumannii Naval- | 390 | 51 |
| | | 82 | | |
| AAC45651.1 | 2393887 | Clostridium pasteurianum DSM | 385 | 51 |
| | | 525 | | |
| YP_007491369.1 | 452211255 | Methanosarcina mazei Tuc01 | 386 | 51 |
| YP_002434746 | 218885425 | Desulfovibrio vulgaris str. | 393 | 50 |
| | | ‘Miyazaki F’ | | |
| YP_005052855 | 374301216 | Desulfovibrio africanus str. | 393 | 49 |
| | | Walvis Bay | | |
| NP_561852.1 | 18309918 | Clostridium perfringens str. 13 | 385 | 49 |
| YP_001447544 | 156976638 | Vibrio campbellii ATCC BAA- | 382 | 49 |
| | | 1116 | | |
| YP_001113612.1 | 134300116 | Desulfotomaculum reducens MI- | 388 | 49 |
| | | 1 | | |
| YP_011618 | 46580810 | Desulfovibrio vulgaris str. | 393 | 49 |
| | | Hildenborough | | |
| ZP_01220157.1 | 90412151 | Photobacterium profundum | 382 | 48 |
| | | 3TCK | | |
| YP_003990729.1 | 312112413 | Geobacillus sp. Y4.1MC1 | 384 | 48 |
| ZP_07335453.1 | 303249216 | Desulfovibrio fructosovorans | 393 | 48 |
| | | JJ | | |
| NP_717107 | 24373064 | Shewanella oneidensis MR-1 | 382 | 48 |
| YP_003310546.1 | 269122369 | Sebaldella termitidis ATCC | 384 | 48 |
| | | 33386 | | |
| ZP_10241531.1 | 390456003 | Paenibacillus peoriae KCTC | 384 | 47 |
| | | 3763 | | |
| YP_001337153.1 | 152972007 | Klebsiella pneumoniae subsp. | 387 | 47 |
| | | pneumoniae MGH 78578 | | |
| YP_026233.1 | 49176377 | Escherichia coli | 383 | 46 |
| YP_694908 | 110799824 | Clostridium perfringens ATCC | 382 | 46 |
| | | 13124 | | |
| YP_725376.1 | 113866887 | Ralstonia eutropha H16 | 366 | 46 |
| YP_001663549 | 167040564 | Thermoanaerobacter sp. X514 | 389 | 45 |
| EKC54576 | 406526935 | human gut metagenome | 384 | 37 |
| YP_001126968.1 | 138896515 | Geobacillus themodenitrificans | 387 | 27 |
| | | NG80-2 | | |

Engineered cells of the disclosure can also express MDH variants. In some cases, variants having less than 100% identity of SEQ ID NO:4 can be generated by sequence alignment of SEQ ID NO:4 with other known methanol dehydrogenases or alcohol dehydrogenases to identify regions that are conserved and/or important for enzymatic functioning of the protein. Once these regions are identified, the methanol dehydrogenase can be modified at one or more amino acid locations outside of these conserved regions. Therefore, the methanol dehydrogenase can have one or more amino acid substitutions, deletions, or additions which cause the sequence to vary from a native methanol dehydrogenase while retaining certain sequence features. Table 8 is a table of pairwise sequence identity of various methanol dehydrogenases including Bacillus methanolicus MGA3 MeDH (SEQ ID NO: 4).

TABLE 8
Pairwise alignment (% ID)

| | Bacillus | Bacillus | Bacillus | | |
|---|---|---|---|---|---|
| | methanolicus | methanolicus | methanolicus | Lysinibacillus | Clostridium |
| PHI | MGA3¹ | MGA3² | PB1 | fusiformis | perfringens str. 13 |
| Bacillus methanolicus MGA3¹ | 100 | 62 | 60.7 | 58.4 | 48.7 |
| Bacillus methanolicus MGA3² | 62 | 100 | 92.7 | 72.2 | 53.2 |
| Bacillus methanolicus PB1 | 60.7 | 92.7 | 100 | 72.2 | 53.5 |
| Lysinibacillus fusiformis | 58.4 | 72.2 | 72.2 | 100 | 51.2 |
| Clostridium perfringens str. 13 | 48.7 | 53.2 | 53.5 | 51.2 | 100 |

¹EIJ77596.1
²EIJ83020.1

Such variants may provide increased catalytic activity, such as increased conversion of methanol to formaldehyde. Exemplary variants of Bacillus methanolicus MGA3 MeDH (SEQ ID NO: 1) are described in International Patent Application No. PCT/US2014/059135, the disclosure of which is incorporated herein. Exemplary amino acid substitutions of SEQ ID NO: 4 include, but are not limited to, those as follows: S11T, D38N, H42Q, E48D, N53I, E56K, D60E, V61A, I63F, P65Q, D70N, P71I, P71T, P71V, T74S, D81G, K84R, E86K, N87K, I94V, S99P, S99T, A103V, I106L, G107S, L108V, L108W, V109Y, N112K, N112R, R115H, I116F, N117D, N117Q, N117Y, Q120H, Q120R, G121A, G121D, G121E, G121L, G121M, G121R, G121S, G121T, G121V, G121W, G121Y, V122A, V122P, N123D, N123I, N123L, N123R, N123Y, S124I, S124L, S124R, V125C, V125G, V125W, E126G, E126V, K127C, K127R, P128A, P128R, P128S, V129A, V129M, V129P, V129S, V130F, V130I, V130Y, A134T, S143T, T145M, T146N, S147R, L148A, L148F, L148G, L148I, L148T, L148V, L148W, A149L, A149M, A149T, A149V, V150A, V150I, T152M, A155V, K157N, V158E, V158H, V158K, V158W, P161A, P161G, P161Q, P161S, P161V, I163F, I163N, I163Q, I163T, D164G, D164N, E165G, K181R, A184T, L186M, T190A, T190S, I199V, Q217K, L226M, G256C, Q267H, G269S, G270M, G270S, G270Y, T296S, R298H, A300T, I302V, G312V, A316V, I323M, F333L, P336L, S337C, G343D, V344A, V344G, K345E, E350K, K354M, N355D, N355I, N355K, E358G, V360A, V360G, V360K, V360R, V360S, C361N, C361R, Q363K, and K379M.

Other exemplary amino acid substitutions of SEQ ID NO: 1 include: D38N, D60E, P71I, P71V, N87K, S99T, A103V, G107S, L108V, L108W, V109Y, R115H, I116F, N117D, N117Q, G121D, G121E, G121L, G121M, G121R, G121S, G121T, G121V, G121W, G121Y, V122P, N123D, N123I, N123L, N123R, N123Y, S124I, S124L, V125C, V125G, V125W, E126G, K127C, K127R, P128A, P128R, P128S, V129A, V129M, V129P, V129S, V130F, V130I, V130Y, A134T, S143T, T146N, A149L, A149M, A149T, A149V, V150A, K157N, V158E, V158H, V158K, V158W, I163Q, D164N, Q267H, G270M, G270S, G270Y, K345E, N355D, V360G, V360K, V360R, V360S, C361R.

Engineered cells of the disclosure can also express a MeDH activator protein. A MeDH activator protein can activate a MeDH enzyme by providing hydrolytic removal of a nicotinamide mononucleotide (NMN) moiety of the NAD cofactor. MeDH activator is active in the presence of magnesium ions and is also able to use ADP-ribose. (Kloosterman, H., et al. (2002) J Biol Chem. 277:34785-34792).

The engineered fusion protein can include a polypeptide sequence based on MeDH activator proteins, such as, activators of Bacillus methanolicus MGA3 (WP_004435441.1) and Bacillus methanolicus PB1 (WP_004437560.1).

Engineered cells of the disclosure can also express a fusion protein that includes methanol dehydrogenase activity and at least one other activity that promotes formaldehyde fixation. For example, the fusion can include activities which promote the conversion of methanol to formaldehyde and then from formaldehyde to a ketose phosphate such as hexulose 6-phosphate, or fructose-6-phosphate. Alternatively, for example, the fusion can include activities which promote the conversion of methanol to formaldehyde and then from formaldehyde to dihydroxyacetone (DHA) and glycerladehyde-3-phosphate (G3P), and then to fructose-6-phosphate. MeDH-containing fusion protein that includes one or more of the following sequences: a HPS sequence, a PHI sequence, and/or an MeDH activator sequence (ACT). Use of such fusions can promote methanol uptake, such as by increased efficiency of fixation of formaldehyde into ketose phosphate compounds as a result of the fusion.

Exemplary fusion proteins include those designated MeDH-ACT-PHI, MeDH-ACT-HPS, HPS-MeDH-ACT, ACT-MeDH-PHI-HPS, ACT-MeDH-HPS-PHI, HPS-PHI-MeDH-ACT, PHI-HPS-MeDH-ACT, and ACT-MeDH(P1)-HPS-MeDH(P2). Such fusion proteins are described in WO2017/075208 (Barton et al.) the disclosure of which is incorporated herein by reference.

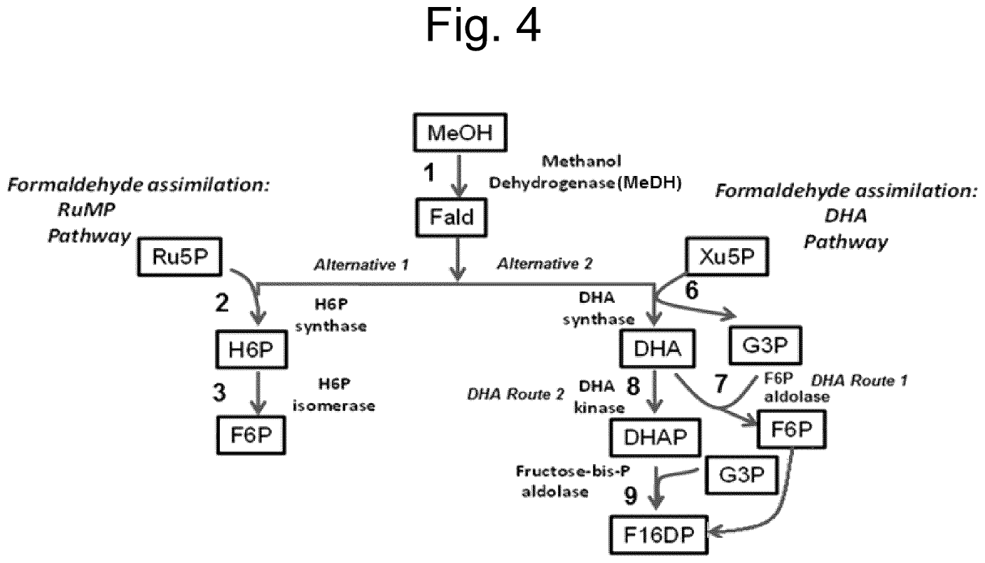

With reference to FIG. 4, another exemplary pathway that involves the detoxification and assimilation of formaldehyde produced from the oxidation of methanol proceeds through dihydroxyacetone. Dihydroxyacetone synthase is a transketolase that first transfers a glycoaldehyde group from xylulose-5-phosphate to formaldehyde, resulting in the formation of dihydroxyacetone (DHA) and glyceraldehyde-3-phosphate (G3P), which is an intermediate in glycolysis. The DHA obtained from DHA synthase can be then further phosphorylated to form DHA phosphate by a DHA kinase. DHAP can be assimilated into glycolysis, e.g. via isomerization to G3P, and several other pathways. Alternatively, DHA and G3P can be converted by fructose-6-phosphate aldolase to form fructose-6-phosphate (F6P)

The dihydroxyacetone synthase enzyme in Candida boidinii uses thiamine pyrophosphate and Mg²⁺ as cofactors and is localized in the peroxisome. The enzyme from the methanol-growing carboxydobacterium, Mycobacter sp. strain JC1 DSM 3803, was also found to have DHA synthase and kinase activities (Ro et al., 1997, JBac 179(19):6041-7). DHA synthase from this organism also has similar cofactor requirements as the enzyme from C. boidinii. The Kₘs for formaldehyde and xylulose 5-phosphate were reported to be 1.86 mM and 33.3 microM, respectively. Several other mycobacteria, excluding only Mycobacterium tuberculosis, can use methanol as the sole source of carbon and energy and are reported to use dihydroxyacetone synthase (Part et al., 2003, JBac 185(1):142-7.

TABLE 9

| Protein | GenBank ID | GI number | Organism |
|---|---|---|---|
| DAS1 | AAC83349.1 | 3978466 | Candida boidinii |
| HPODL_2613 | EFW95760.1 | 320581540 | Ogataea |
| | | | parapolymorpha DL-1 |
| | | | (Hansenula polymorpha |
| | | | DL-1) |
| | AAG12171.2 | 18497328 | Mycobacter sp. strain |
| | | | JC1 DSM 3803 |

Fructose-6-phosphate aldolase (F6P aldolase) can catalyze the combination of dihydroxyacetone (DHA) and glyceraldehyde-3-phosphate (G3P) to form fructose-6-phosphate. This activity was recently discovered in E. coli and the corresponding gene candidate has been termed fsa (Schurmann and Sprenger, J. Biol. Chem., 2001, 276(14), 11055-11061). The enzyme has narrow substrate specificity and cannot utilize fructose, fructose 1-phosphate, fructose 1,6-bisphosphate, or dihydroxyacetone phosphate. It can however use hydroxybutanone and acetol instead of DHA. The purified enzyme displayed a Vₘₐₓ of 7 units/mg of protein for fructose 6-phosphate cleavage (at 30 degrees C., pH 8.5 in 50 mm glycylglycine buffer). For the aldolization reaction a Vₘₐₓ of 45 units/mg of protein was found; Kₘ values for the substrates were 9 mM for fructose 6-phosphate, 35 mM for dihydroxyacetone, and 0.8 mM for glyceraldehyde 3-phosphate. The enzyme prefers the aldol formation over the cleavage reaction.

The selectivity of the E. coli enzyme towards DHA can be improved by introducing point mutations. For example, the mutation A129S improved reactivity towards DHA by over 17 fold in terms of Kcat/Km (Gutierrez et al., Chem Commun (Camb), 2011, 47(20), 5762-5764). The same mutation reduced the catalytic efficiency on hydroxyacetone by more than 3 fold and reduced the affinity for glycoaldehyde by more than 3 fold compared to that of the wild type enzyme (Castillo et al., Advanced Synthesis & Catalysis, 352(6), 1039-1046). Genes similar to fsa have been found in other genomes by sequence homology. Some exemplary gene candidates have been listed in Table 10 below.

TABLE 10

| | Protein | | |
|---|---|---|---|
| Gene | accession no. | GI number | Organism |
| fsa | AAC73912.2 | 87081788 | Escherichia coli K12 |
| talC | AAC76928.1 | 1790382 | Escherichia coli K12 |
| fsa | WP_017209835.1 | 515777235 | Clostridium beijerinckii |
| DR_1337 | AAF10909.1 | 6459090 | Deinococcus radiodurans |
| | | | R1 |
| talC | NP_213080.1 | 15605703 | Aquifex aeolicus VF5 |
| MJ_0960 | NP_247955.1 | 15669150 | Methanocaldococcus |
| | | | janaschii |
| mipB | NP_993370.2 | 161511381 | Yersinia pestis |

As described below, there is an energetic advantage to using F6P aldolase in the DHA pathway. The assimilation of formaldehyde formed by the oxidation of methanol can proceed either via the dihydroxyacetone (DHA) pathway or the Ribulose monophosphate (RuMP) pathway. In the RuMP pathway, formaldehyde combines with ribulose-5-phosphate to form F6P. F6P is then either metabolized via glycolysis or used for regeneration of ribulose-5-phosphate to enable further formaldehyde assimilation. Notably, ATP hydrolysis is not required to form F6P from formaldehyde and ribulose-5-phosphate via the RuMP pathway.

In contrast, in the DHA pathway, formaldehyde combines with xylulose-5-phosphate (X5P) to form dihydroxyacetone (DHA) and glyceraldehyde-3-phosphate (G3P). Some of the DHA and G3P must be metabolized to F6P to enable regeneration of xylulose-5-phosphate. In the standard DHA pathway, DHA and G3P are converted to F6P by three enzymes: DHA kinase, fructose bisphosphate aldolase, and fructose bisphosphatase. The net conversion of DHA and G3P to F6P requires ATP hydrolysis as described below. First, DHA is phosphorylated to form DHA phosphate (DHAP) by DHA kinase at the expense of an ATP. DHAP and G3P are then combined by fructose bisphosphate aldolase to form fructose-1,6-diphosphate (FDP). FDP is converted to F6P by fructose bisphosphatase, thus wasting a high energy phosphate bond.

A more ATP efficient sequence of reactions is enabled if DHA synthase functions in combination with F6P aldolase as opposed to in combination with DHA kinase, fructose bisphosphate aldolase, and fructose bisphosphatase. F6P aldolase enables direct conversion of DHA and G3P to F6P, bypassing the need for ATP hydrolysis. Overall, DHA synthase when combined with F6P aldolase is identical in energy demand to the RuMP pathway. Both of these formaldehyde assimilation options (i.e., RuMP pathway, DHA synthase+F6P aldolase) are superior to DHA synthase combined with DHA kinase, fructose bisphosphate aldolase, and fructose bisphosphatase (exogenous glpX) in terms of ATP demand.

Transaldolase (EC 2.2.1.2) plays a role in the balance of metabolites in the pentose-phosphate pathway. There are two closely related transaldolase genes in E. coli, encoded by talA and ta/B. In E. coli, TalB (Transaldolase B; Uniprot P0A867) catalyzes the interconversion of sedoheptulose 7-phosphate and D-glyceraldehyde 3-phosphate to D-erythrose 4-phosphate+D-fructose 6-phosphate. Homologues of these genes can be found in other microbes including C. glutamicum, S. cerevisiae, Pseudomonas putida, Bacillus subtilis. In embodiments of the disclosure, a component of autocatalytic RuMP cycle for synthetic methylotrophy includes regeneration of C5 from C7 in the form of sedoheptulose-7-phosphate (S7P) and C3 glyceraldehyde-3-phosphate (G3P). There can be variants of the RuMP cycle based on how the C7 compound S7P is generated. One is a transaldolase variant of the RuMP cycle where the transaldoalse genes talA, talB, and/or talC is used to generate S7P. Another variant is the sedoheptulose bisphosphatase (SBPase) variant where a fructose bisphosphate aldolase (Qba) is used to condense DHAP and E4P into sedoheptulose-1,7-bisphosphate (SBP) which is then dephosphorylated to S7P using the SBPase enzyme encoded by the gene glpX. See FIGS. 5 and 6. While the transaldolase variant of the RuMP cycle provides an energetic advantage over the SBPase variant of the RuMP cycle, the SBPase variant provides a substantial thermodynamic advantage with the glpX reaction being highly thermodynamically favourable. Exemplary candidates for the SBPase variant of the RuMP cycle include the fba (EU77593.1, EU77616.1) and glpX genes (ZP_11548894, WP_003352248.1) from Bacillus methanolicus.

Due to this thermodynamic advantage, and in preferred aspects of the disclosure, an engineered microorganism includes the SBPase variant of the RuMP cycle wherein exogenous fba and glpX genes are overexpressed and the native transalodolase genes (ta/A, talB, talC) are attenuated or eliminated. See FIGS. 5 and 6. This can also be combined with the introduction and activity of the exogenous gapN expressed in the cells, and optionally the deletion or attenuation of GapA activity. In such engineered cells, the introduction of gapN can improve pool sizes of the RuMP cycle metabolites and the SBPase variant of the RuMP cycle in the form of exogenous fba and glpX with the deletion or attenuation of talA, talB, talC provides the thermodynamic driving force to achieve an autocatalytic RuMP cycle.

In embodiments, the engineered microorganism can include a modification that attenuates or eliminates an endogenous activity of a fructose-bisphosphate aldolase combined with the overexpression of exogenous fructose-bisphosphate aldolase (fba2).

The enzyme fructose-bisphosphate aldolase catalyzes the conversion of fructose 1,6-bisphosphate to two C3 phosphate compounds which are dihydroxyacetone phosphate (DHAP or glycerone-phosphate) and glyceraldehyde 3-phosphate (G3P). In E. coli. this activity is catalyzed by either one of the two genes fbaA or fbaB. The E. coli fbaA and fbaB are known to be inhibited by high levels of its C3 products G3P and DHAP. However, according to the disclosure, high pool sizes of G3P and DHAP are desirably maintained for synthetic methylotrophy. In order to achieve this balance, embodiments of engineered microorganisms can include modifications to delete or attenuate native fructose-bisphosphate aldolase activity and introduction of an exogenous fructose-bisphosphate aldolase, such as one from Bacillus.

For example, the fructose-bisphosphate aldolase fba2 from Bacillus methanolicus PB1, or a homolog or variant thereof, can be introduced into the engineered microorganism. The engineered microorganism can express a fba sequence that is related to Bacillus methanolicus PB1 fba2, such as a Bacillus methanolicus PB1 fba2 homolog or variant. For example, the fba sequence can be 50% or greater, 60% or greater, 70% or greater, 80% or greater, 85% or greater, 90% or greater, or 95% or greater identity to Bacillus methanolicus PB1 fba2 (SEQ ID NO:9).

This can be combined with a microorganism with exogenous GapN and attenuated/deleted GapA activity.

Other exemplary fructose-bisphosphate aldolase genes that can be introduced into the engineered microorganism are listed in Table 11 below:

TABLE 11

| | | | |
|---|---|---|---|
| WP_003346852.1 | WP_066310121.1 | WP_042354391.1 | WP_098260191.1 |
| WP_003351726.1 | WP_095243208.1 | WP_019153989.1 | WP_045515920.1 |
| WP_101661177.1 | WP_079506262.1 | WP_101178886.1 | WP_076759362.1 |
| WP_038537846.1 | WP_066205853.1 | WP_098571546.1 | WP_063254575.1 |
| WP_057772794.1 | WP_053433445.1 | WP_070877989.1 | WP_044392746.1 |
| WP_053368407.1 | WP_101548883.1 | WP_066073012.1 | WP_088090227.1 |
| WP_026585103.1 | WP_102264606.1 | WP_090634787.1 | WP_016202508.1 |
| WP_102273593.1 | WP_063389040.1 | WP_029283302.1 | WP_098932372.1 |
| WP_101665800.1 | WP_053403739.1 | WP_066056862.1 | WP_098795425.1 |
| WP_066367433.1 | WP_066389558.1 | WP_071393265.1 | WP_046130283.1 |
| WP_101579374.1 | WP_009792596.1 | WP_069938500.1 | WP_075983208.1 |
| WP_077215087.1 | WP_066234213.1 | WP_047940761.1 | WP_078432014.1 |
| WP_053478524.1 | WP_053597680.1 | WP_107920563.1 | WP_071461009.1 |
| WP_027323492.1 | WP_009332285.1 | WP_098527749.1 | WP_031538951.1 |
| WP_066398310.1 | WP_101581996.1 | WP_066235121.1 | WP_026563084.1 |
| WP_075686757.1 | WP_090832678.1 | WP_059352025.1 | WP_006639440.1 |
| WP_024031049.1 | WP_046588513.1 | WP_046179059.1 | WP_006837435.1 |
| WP_080843816.1 | WP_043930083.1 | WP_102230461.1 | WP_050183379.1 |
| WP_057760136.1 | WP_015596100.1 | WP_066267408.1 | WP_046174604.1 |
| WP_066296675.1 | SHP69806.1 | WP_044894355.1 | |
| WP_095312127.1 | WP_058005248.1 | WP_101567317.1 | |
| WP_048827259.1 | WP_095371563.1 | WP_101356082.1 | |
| WP_059171825.1 | WP_072578145.1 | WP_066248707.1 | |
| WP_066093948.1 | WP_090762628.1 | WP_059283615.1 | |
| WP_048009440.1 | WP_066189121.1 | WP_041964633.1 | |
| WP_019380596.1 | WP_007087445.1 | WP_023613550.1 | |
| WP_066442299.1 | WP_095301761.1 | WP_101648384.1 | |

In embodiments, the engineered microorganism includes modifications that attenuates or eliminates an endogenous activity of a fructose-bisphosphate aldolase, and that introduces an exogenous fructose-bisphosphate aldolase.

In embodiments, the engineered microorganism can include a modification that introduces an exogenous activity of a triose-phosphate isomerase (tpi, such as tpiA). Exemplary triose-phosphate isomerase genes that can be introduced into the engineered microorganism are listed in Table 22 below:

TABLE 22
Exemplary tpiA homologs - Accession Number

| | | | |
|---|---|---|---|
| WP_049625147.1 | WP_048622612.1 | WP_119544996.1 | WP_061465550.1 |
| WP_057984053.1 | WP_066442636.1 | WP_098797852.1 | WP_048688041.1 |
| NP_418354.1 | WP_066258742.1 | WP_099352518.1 | WP_066290710.1 |
| WP_003352090.1 | WP_080844080.1 | WP_133312649.1 | WP_016203861.1 |
| WP_003349425.1 | WP_071460731.1 | WP_028403379.1 | WP_106027029.1 |
| WP_125927893.1 | WP_066065633.1 | WP_113969130.1 | WP_120034658.1 |
| WP_101658881.1 | WP_079532208.1 | WP_053403564.1 | WP_130156825.1 |
| WP_101664621.1 | WP_090857112.1 | WP_029284585.1 | WP_110928513.1 |
| WP_101575971.1 | WP_098530337.1 | WP_048015738.1 | WP_033021492.1 |
| WP_026582029.1 | WP_113926855.1 | WP_091704422.1 | WP_096339383.1 |
| WP_101580490.1 | WP_098930974.1 | WP_078414242.1 | WP_046131103.1 |
| WP_110064628.1 | WP_098352031.1 | WP_121663490.1 | PGZ97936.1 |
| PWW29569.1 | WP_058002855.1 | WP_010175280.1 | WP_081160821.1 |
| WP_090833149.1 | WP_040204128.1 | WP_131235750.1 | WP_088008494.1 |
| WP_125481486.1 | WP_071976496.1 | WP_019242991.1 | WP_104058107.1 |
| WP_113883700.1 | WP_126863303.1 | WP_124564487.1 | WP_003397292.1 |
| WP_044390541.1 | WP_108671576.1 | WP_089098086.1 | WP_114896973.1 |
| HAQ06002.1 | WP_075689781.1 | WP_081189305.1 | OUM91325.1 |
| WP_066230398.1 | WP_088089551.1 | WP_015865021.1 | WP_066141854.1 |
| WP_102264288.1 | WP_126405446.1 | WP_097159265.1 | WP_097961009.1 |
| WP_041967473.1 | WP_121617301.1 | WP_095480439.1 | WP_020155099.1 |
| WP_107920638.1 | WP_061809791.1 | WP_062677276.1 | WP_063233964.1 |
| WP_108069989.1 | WP_101635495.1 | WP_133376450.1 | WP_116516504.1 |
| WP_079506869.1 | WP_034764215.1 | WP_101568078.1 | WP_098864843.1 |
| WP_053433745.1 | WP_044893559.1 | WP_028396129.1 | WP_013085385.1 |
| WP_066366023.1 | WP_060672585.1 | WP_066419543.1 | WP_089361938.1 |
| WP_095310191.1 | WP_101643715.1 | WP_125907547.1 | WP_095371908.1 |
| WP_061791816.1 | WP_080860079.1 | WP_041113450.1 | WP_061141152.1 |
| WP_076258936.1 | WP_041087339.1 | WP_100332287.1 | WP_097899256.1 |
| WP_023613939.1 | WP_042353961.1 | WP_136378456.1 | WP_098060328.1 |
| WP_035329301.1 | WP_059172080.1 | WP_116353411.1 | WP_098308454.1 |
| WP_046522451.1 | WP_119709961.1 | WP_041094811.1 | WP_035430455.1 |
| WP_066387219.1 | WP_003353516.1 | WP_077619443.1 | WP_098779423.1 |
| WP_043930314.1 | WP_117306959.1 | WP_102232454.1 | WP_035066254.1 |
| WP_026575982.1 | WP_028393606.1 | WP_059283432.1 | WP_057957776.1 |
| WP_053597872.1 | WP_136832879.1 | WP_042350706.1 | WP_057215857.1 |
| WP_009331815.1 | WP_132092731.1 | GAE47365.1 | WP_026684780.1 |
| WP_127488577.1 | WP_119113030.1 | WP_063384768.1 | WP_063193279.1 |
| WP_053360533.1 | WP_100333252.1 | WP_066175408.1 | WP_013860187.1 |
| WP_050616384.1 | WP_095246679.1 | WP_085787813.1 | WP_098185504.1 |
| WP_009793077.1 | WP_048013384.1 | WP_110112470.1 | WP_113803922.1 |
| WP_114746353.1 | WP_044741433.1 | WP_026559622.1 | WP_116366259.1 |
| WP_118921487.1 | WP_101647122.1 | WP_134375855.1 | WP_001231047.1 |
| WP_032086617.1 | SHT19397.1 | WP_004892489.1 | WP_098684976.1 |
| WP_034674239.1 | WP_115451363.1 | WP_066329182.1 | WP_002174167.1 |
| WP_071354652.1 | WP_066322819.1 | WP_066249154.1 | WP_033843086.1 |
| WP_038537244.1 | WP_117322511.1 | WP_012096134.1 | WP_002144919.1 |
| WP_095243508.1 | TMU84398.1 | WP_121446321.1 | WP_001231042.1 |
| WP_048827021.1 | WP_060665410.1 | WP_053536810.1 | WP_098135924.1 |
| WP_019383298.1 | WP_048006747.1 | WP_021093689.1 | WP_070807150.1 |
| WP_121611084.1 | WP_066058820.1 | WP_025727119.1 | WP_049166282.1 |
| WP_027409935.1 | WP_101592182.1 | REJ24619.1 | WP_064467230.1 |
| WP_133333986.1 | WP_075982879.1 | WP_094245233.1 | EEL91210.1 |
| WP_132003505.1 | WP_072578458.1 | GAJ43858.1 | WP_098645443.1 |
| WP_027320678.1 | WP_123919364.1 | WP_099361576.1 | WP_098870165.1 |
| WP_126649451.1 | WP_010196828.1 | WP_076368239.1 | WP_001231046.1 |
| WP_066096909.1 | WP_098439087.1 | WP_078378974.1 | WP_128267196.1 |
| WP_090743218.1 | WP_066149736.1 | WP_003248110.1 | WP_018660881.1 |
| WP_055738838.1 | WP_047969990.1 | WP_041060902.1 | WP_060788254.1 |
| WP_019153676.1 | WP_045518085.1 | WP_128356640.1 | EEK76346.1 |
| WP_078543970.1 | WP_096156668.1 | WP_090948441.1 | WP_002112798.1 |
| WP_090632599.1 | WP_044338273.1 | WP_034309988.1 | WP_061187910.1 |
| WP_071619677.1 | WP_040375829.1 | KYD07778.1 | WP_001231036.1 |
| WP_044892996.1 | WP_095297184.1 | WP_062109228.1 | OUB37012.1 |
| WP_101352804.1 | WP_088019477.1 | SLL35741.1 | WP_003235214.1 |
| WP_066200502.1 | WP_066224972.1 | WP_064552345.1 | WP_120667089.1 |
| TCL47385.1 | WP_071393617.1 | WP_026694507.1 | WP_001231034.1 |
| WP_132949072.1 | WP_046514094.1 | WP_063386958.1 | WP_017153354.1 |
| WP_111645022.1 | WP_064093006.1 | WP_077429731.1 | WP_100062914.1 |
| WP_117327782.1 | WP_094833549.1 | WP_006322696.1 | WP_035190571.1 |
| WP_007083466.1 | WP_015595638.1 | WP_066269699.1 | WP_076541647.1 |
| WP_049683119.1 | WP_121679697.1 | WP_042410586.1 | WP_065224384.1 |
| WP_062185738.1 | WP_064098916.1 | WP_017435719.1 | WP_069150333.1 |
| WP_066314500.1 | WP_070878079.1 | WP_095258588.1 | WP_053348617.1 |
| WP_026567800.1 | WP_102273864.1 | WP_043906652.1 | WP_137016647.1 |
| WP_077215437.1 | WP_100401758.1 | WP_078431648.1 | WP_019394944.1 |
| WP_098260028.1 | WP_039230741.1 | WP_111617058.1 | WP_016079761.1 |
| WP_042462893.1 | WP_035404544.1 | WP_057776373.1 | WP_013059700.1 |
| WP_053368188.1 | WP_057763397.1 | WP_101223713.1 | WP_098836424.1 |
| WP_098572329.1 | WP_090761376.1 | WP_047943786.1 | WP_063670679.1 |
| WP_024031175.1 | WP_053477630.1 | WP_136358937.1 | WP_016718893.1 |
| WP_098312709.1 | WP_006838605.1 | WP_054398720.1 | WP_081207163.1 |
| WP_063263115.1 | WP_098492711.1 | WP_050820775.1 | WP_044743781.1 |
| WP_098906044.1 | WP_033012090.1 | WP_098874391.1 | WP_134975096.1 |
| WP_039073025.1 | WP_098563453.1 | WP_098123602.1 | WP_001231039.1 |
| WP_124051235.1 | WP_018783331.1 | WP_014478011.1 | WP_071392302.1 |
| WP_074553722.1 | WP_043977318.1 | WP_063164796.1 | WP_098758980.1 |
| WP_001231038.1 | WP_010899687.1 | AFQ59244.1 | WP_001990120.1 |
| WP_061574301.1 | WP_057239856.1 | WP_048566659.1 | WP_016937890.1 |
| WP_017561245.1 | WP_003201936.1 | WP_098437038.1 | WP_098909730.1 |
| WP_010676552.1 | WP_088113869.1 | WP_128747234.1 | WP_001231044.1 |
| WP_015375781.1 | WP_077671068.1 | WP_026580128.1 | WP_098191861.1 |
| WP_009362287.1 | WP_134378576.1 | WP_033883443.1 | WP_065410424.1 |
| WP_031407162.1 | WP_066106223.1 | WP_079288779.1 | WP_044439339.1 |
| WP_066192014.1 | WP_098163180.1 | ABK87818.1 | WP_002139168.1 |
| WP_017726328.1 | WP_016085605.1 | WP_098149639.1 | WP_105585595.1 |
| WP_098881737.1 | WP_086404129.1 | WP_098009310.1 | WP_098224919.1 |
| WP_061912389.1 | WP_001231041.1 | WP_094911130.1 | WP_098582415.1 |
| WP_014097474.1 | WP_071708738.1 | WP_043925539.1 | WP_073543968.1 |
| WP_031540679.1 | WP_002159914.1 | WP_100664262.1 | WP_124047914.1 |
| WP_057244845.1 | WP_137023276.1 | ANC33038.1 | WP_060749269.1 |
| WP_061570307.1 | WP_074601757.1 | WP_069838710.1 | WP_081133124.1 |
| WP_055441732.1 | KZM58399.1 | WP_059037336.1 | WP_025949885.1 |
| WP_098087536.1 | WP_113303501.1 | WP_071728240.1 | WP_042513393.1 |
| WP_128805263.1 | WP_098578860.1 | WP_098946395.1 | WP_033673190.1 |
| WP_053430308.1 | WP_016112483.1 | WP_001231037.1 | WP_098562107.1 |
| WP_057912038.1 | WP_025148228.1 | WP_001231043.1 | WP_071710099.1 |
| WP_118043288.1 | WP_088024791.1 | WP_020756339.1 | WP_098481312.1 |
| WP_129447198.1 | WP_071389124.1 | REJ13911.1 | WP_049664732.1 |
| WP_098053973.1 | WP_024713486.1 | WP_057998856.1 | WP_097794489.1 |
| WP_113769676.1 | WP_002124085.1 | WP_002016069.1 | AFJ63586.1 |
| WP_097895391.1 | WP_137011592.1 | WP_097840938.1 | WP_071736583.1 |
| WP_056524524.1 | WP_003185464.1 | WP_129705348.1 | WP_039075461.1 |
| WP_061686728.1 | WP_020453003.1 | WP_131887487.1 | WP_003151618.1 |
| WP_049669517.1 | WP_046218016.1 | WP_057275308.1 | WP_087991169.1 |
| WP_118498271.1 | WP_038413800.1 | WP_039810889.1 | WP_045385091.1 |
| WP_116821155.1 | WP_003243394.1 | WP_098094278.1 | REJ30438.1 |
| WP_129507564.1 | WP_019715758.1 | WP_061679291.1 | WP_098544968.1 |
| WP_016136934.1 | WP_034634933.1 | WP_020961189.1 | WP_130572861.1 |
| WP_076788798.1 | WP_075421549.1 | WP_102956614.1 | WP_098374188.1 |
| WP_070170654.1 | WP_010328643.1 | TKH99978.1 | WP_105981242.1 |
| WP_001231040.1 | WP_003219962.1 | EDR92067.1 | WP_088232033.1 |
| WP_106073398.1 | WP_103749369.1 | WP_033015090.1 | WP_017418891.1 |
| WP_061047067.1 | WP_104849558.1 | WP_010331914.1 | WP_001231045.1 |
| WP_025907462.1 | WP_063094158.1 | WP_106074644.1 | WP_070082401.1 |
| WP_082998444.1 | | | |

Methylglyoxal synthase (EC 4.2.3.3), also known as glycerone-phosphate phospho-lyase, is an enzyme that catalyzes the formation of methylglyoxal and phosphate from dihydroxyacetone phosphate. In E. coli, methylglyoxal synthase is encoded by mgsA (Uniprot P0A731).

Optionally, the engineered microorganism can include a modification that can attenuate or eliminate the activity of a methylglyoxal synthase, such as E. coli mgsA. In turn this can result in an increase in the pools of dihydroxyacetone phosphate, which in turn can be converted to G3P or sedoheptulose 1,7, which can complement the introduction and activity of the exogenous gapN expressed in the cells, and optionally the deletion or attenuation of gapA activity.

Deoxyribose phosphate aldolase (EC 4.1.2.4) catalyzes a reversible aldol reaction between acetaldehyde and D-glyceraldehyde 3-phosphate to generate 2-deoxy-D-ribose 5-phosphate. In E. coli, Doxyribose phosphate aldolase is encoded by deoC (Uniprot P0A6L0).

Optionally, the engineered microorganism can include a modification that can attenuate or eliminate the activity of the deoxyribose phosphate aldolase, such as E. coli deoC. In turn this can result in an increase in the pools of G3P, which can complement the introduction and activity of the exogenous gapN expressed in the cells, and optionally the deletion or attenuation of gapA activity.

With reference to FIG. 5, in embodiments, the engineered microorganism can include a modification that attenuates or eliminates an endogenous activity of an ATP-dependent 6-phosphofructokinase. This enzyme can catalyze the phosphorylation of D-fructose 6-phosphate to fructose 1,6-bisphosphate by ATP. In E. coli, the ATP-dependent 6-phosphofructokinase isozyme 1 is encoded by pfkA, which can be modified to delete or attenuate phosphofructokinase enzymatic activity.

In embodiments, the engineered microorganism can include a modification that introduces an exogenous ATP-dependent 6-phosphofructokinase, such as one from Bacillus. For example, the ATP-dependent 6-phosphofructokinase Pfk2 from Bacillus methanolicus MGA3, or a homolog or variant thereof, can be introduced into the engineered microorganism. The engineered microorganism can express a pfk sequence that is related to Bacillus methanolicus MGA3 pfk2, such as a Bacillus methanolicus MGA pfk2 homolog or variant. For example, the pfk sequence can be 50% or greater, 60% or greater, 70% or greater, 80% or greater, 85% or greater, 90% or greater, or 95% or greater identity to Bacillus methanolicus MGA3 pfk2 (SEQ ID NO:5).

Other exemplary ATP-dependent 6-phosphofructokinase genes that can be introduced into the engineered microorganism are listed in Table 12 below.

TABLE 12

| | | NCBI accession |
|---|---|---|
| Gene | Organism | number |
| pfk | Amycolatopsis methanolica 239 | AIJ24607.1 |
| pfk | Geobacillus thermodenitrificans NG80-2 | WP_008880861.1 |
| pfk2 | Bacillus methanolicus MGA3 | WP_003347446.1 |
| pfkB | Escherichia coli K-12 MG1655 | NP_416237.3 |

In embodiments, the engineered microorganism includes modifications that attenuates or eliminates an endogenous activity of an ATP-dependent 6-phosphofructokinase, and that introduces an exogenous ATP-dependent 6-phosphofructokinase. Optionally, these modifications can be combined with the overexpression of exogenous GapN and the attenuation/deletion of GapA.

With reference to FIG. 5, in embodiments, the engineered microorganism can include a modification that attenuates or eliminates an endogenous activity of a ribulose-phosphate 3-epimerase.

This enzyme can catalyze the reversible epimerization of D-ribulose 5-phosphate to D-xylulose 5-phosphate. In E. coli, ribulose-phosphate 3-epimerase is encoded by rpe, which can be modified to delete or attenuate ribulose-phosphate 3-epimerase activity.

In embodiments, the engineered microorganism can include a modification that introduces an exogenous ribulose-phosphate 3-epimerase, such as one from Bacillus. For example, the ribulose-phosphate 3-epimerase rpe from Bacillus methanolicus PB1, or a homolog or variant thereof, can be introduced into the engineered microorganism. The engineered microorganism can express a rpe sequence that is related to Bacillus methanolicus MGA3 rpe, such as a Bacillus methanolicus MGA rpe homolog or variant. For example, the rpe sequence can be 30% or greater, 40% or greater, 50% or greater, 60% or greater, 70% or greater, 80% or greater, 85% or greater, 90% or greater, or 95% or greater identity to Bacillus methanolicus MGA3 rpe (SEQ ID NO:6).

Exemplary ribulose-phosphate 3-epimerase genes that can be introduced into the engineered microorganism are listed in Table 13 below.

TABLE 13

| | | NCBI accession |
|---|---|---|
| Gene | Organism | number |
| rpe | Amycolatopsis methanolica 239 | AIJ24612.1 |
| rpe | Amycolatopsis methanolica 239 | AIJ24222.1 |
| rpe | Bacillus methanolicus MGA3 plasmid | WP_003349832.1 |
| | pBM19 | |
| rpe | Bacillus methanolicus PB1 | WP_003352245.1 |
| rpe | Geobacillus thermodenitrificans NG80-2 | WP_008878632.1 |
| rpe | Methylobacillus flagellatus | ABE50737.1 |
| rpe | Methylophilus methylotrophus ATCC | WP_018987244.1 |
| | 53528 | |

In embodiments, the engineered microorganism includes modifications that attenuates or eliminates an endogenous activity of a ribulose-phosphate 3-epimerase, and that introduces an exogenous ribulose-phosphate 3-epimerase.

With reference to FIG. 5, in embodiments, the engineered microorganism can include a modification that attenuates or eliminates an endogenous activity of a ribose-5-phosphate isomerase.

This enzyme can catalyze the reversible conversion of ribose-5-phosphate to ribulose 5-phosphate. In E. coli, the ribose-5-phosphate isomerase A enzyme is encoded by rpiA, and ribose-5-phosphate isomerase B enzyme is encoded by rpiB, and either or both rpiA and/or rpiB can be modified to delete or attenuate ribose-5-phosphate isomerase activity.

In embodiments, the engineered microorganism can include a modification that introduces an exogenous ribose-5-phosphate isomerase, such as one from Bacillus. For example, the ribose-5-phosphate isomerase rpiB from Bacillus methanolicus PB1, or a homolog or variant thereof, can be introduced into the engineered microorganism. The engineered microorganism can express a rpi sequence that is related to Bacillus methanolicus MGA3 rpiB, such as a Bacillus methanolicus MGA rpiB homolog or variant. For example, the rpi sequence can be 30% or greater, 40% or greater, 50% or greater, 60% or greater, 70% or greater, 80% or greater, 85% or greater, 90% or greater, or 95% or greater identity to Bacillus methanolicus MGA3 rpiB (SEQ ID NO:7). Other exemplary ribose-5-phosphate isomerase genes that can be introduced into the engineered microorganism are listed in Table 14 below.

TABLE 14

| | | NCBI accession |
|---|---|---|
| Gene | Organism | number |
| rpi | Amycolatopsis methanolica 239 | AIJ26616.1 |
| rpi | Geobacillus thermodenitrificans NG80-2 | WP_008880705.1 |
| rpi | Methylobacillus flagellatus | ABE48400.1 |
| rpi | Methylophilus methylotrophus ATCC 53528 | WP_018987608.1 |
| rpiB | Amycolatopsis methanolica 239 | AIJ24621.1 |
| rpiB | Bacillus methanolicus MGA3 | WP_003346829.1 |
| rpiB | Bacillus methanolicus PB1 | WP_003351746.1 |
| rpiB | Methylobacillus flagellatus | ABE49230.1 |

In embodiments, the engineered microorganism includes modifications that attenuates or eliminates an endogenous activity of a ribose-5-phosphate isomerase, and that introduces an exogenous ribose-5-phosphate isomerase.

With reference to FIG. 5, in embodiments, the engineered microorganism can include a modification that attenuates or eliminates an endogenous activity of a transketolase. This enzyme can catalyze the reversible transfer of a two-carbon ketol group from sedoheptulose-7-phosphate to glyceraldehyde-3-phosphate, producing xylulose-5-phosphate and ribose-5-phosphate. In E. coli, the transketolase 1 enzyme is encoded by tktA, and the transketolase 2 enzyme is encoded by tktB, and either or both tktA and/or tktB can be modified to delete or attenuate transketolase activity.

In embodiments, the engineered microorganism can include a modification that introduces an exogenous transketolase, such as one from Bacillus. For example, the transketolase tkt2 from Bacillus methanolicus PB1, or a homolog or variant thereof, can be introduced into the engineered microorganism. The engineered microorganism can express a tkt sequence that is related to Bacillus methanolicus PB) tkt2, such as a Bacillus methanolicus PB) tkt2 homolog or variant. For example, the tkt sequence can be 50% or greater, 60% or greater, 70% or greater, 80% or greater, 85% or greater, 90% or greater, or 95% or greater identity to Bacillus methanolicus PB) tkt2 (SEQ ID NO:8). Other exemplary transketolase genes that can be introduced into the engineered microorganism are listed in Table 15 below

TABLE 15

| | | NCBI accession |
|---|---|---|
| Gene | Organism | number |
| tkt | Amycolatopsis methanolica 239 | AIJ24610.1 |
| tkt2 | Bacillus methanolicus MGA3 | WP_003349240.1 |
| tkt | Bacillus methanolicus MGA3 plasmid | WP_003349838.1 |
| | pBM19 | |
| tkt2 | Bacillus methanolicus PB1 | WP_003350079.1 |
| tkt | Bacillus methanolicus PB1 plasmid pBM20 | WP_003352246.1 |
| tk | Geobacillus thermodenitrificans NG80-2 | WP_008879527.1 |
| tkt | Methylobacillus flagellatus | ABE50516.1 |
| tkt | Methylophilus methylotrophus ATCC 53528 | WP_018987341.1 |
| tkt | Pichia pastoris GS115 | XP_002490261.1 |
| tktA | Amycolatopsis methanolica 239 | AIJ24184.1 |
| tktA1 | Amycolatopsis methanolica 239 | AIJ22337.1 |
| tktA2 | Amycolatopsis methanolica 239 | AIJ22338.1 |

In embodiments, the engineered microorganism includes modifications that attenuates or eliminates an endogenous activity of a transketolase, and that introduces an exogenous transketolase.

In one route 6-phosphofructokinase (EC 2.7.1.11) phosphorylates FMP to fructose 1,6-bisphosphate (FDP). Fructose-bisphosphate aldolase (EC 4.1.2.13) then cleaves FDP into dihydroxy acetone phosphate (DHAP) and glyceraldehyde 3-phosphate.

In another route glucose-6-phosphate isomerase (EC 5.3.1.9) isomerizes FMP to glucose 6-phosphate (GMP). Glucose-6-phosphate 1-dehydrogenase (EC 1.1.1.49) then dehydrogenates GMP to D-glucono-1,5-lactone 6-phosphate which is further dehydrogenated to 6-phospho-gluconate by 6-phosphogluconolactonase (EC 3.1.1.31). Phosphogluconate dehydratase (EC 4.2.1.12) then converts 6-phospho-gluconate to 2-keto-3-deoxy-6-phospho-D-gluconate (KDPG). Subsequently, KDPG aldolase (EC 4.1.2.14) cleaves KDPG into glyceraldehyde 3-phosphate and pyruvate. Pyruvate and DHAP formed through this pathway can be used in cellular pathways for the synthesis of biomolecules.

Optionally, the engineered microorganism can include a modification that can attenuate or eliminate the activity of a KHG/KDPG aldolase. In E. coli KHG/KDPG aldolase is encoded by the eda gene (Uniprot P0A955). Optionally, the engineered microorganism can include a modification that can attenuate or eliminate the activity of a glucose-6-phosphate 1-dehydrogenase. In E. coli glucose-6-phosphate 1-dehydrogenase is encoded by the zwf gene (Uniprot P0AC53). The glucose-6-phosphate 1-dehydrogenase reaction encoded by the zwf gene can provide a dissimilatory pathway due to the decarboxylation catalyzed by the GND reaction, and in turn result in suboptimal yield of biosynthetic pathways from methanol. However, the dissimilatory RuMP pathway via zwf may provide a route for formaldehyde detoxification to balance formaldehyde assimilation via assimilatory RuMP. In addition to formaldehyde dissimilation via pentose phosphate pathway (zwf), bacteria also possess a linear formaldehyde dissimilation pathway, where formaldehyde is oxidized to formate and then to CO₂. This pathway is encoded by endogenous genes frmR, frmA, frmB in E. coli. Additionally, some methylotrophs also possess genes such as folD, fhs, and fdhA to encode for this linear dissimilation pathway. The advantage of the formaldehyde dissimilation via pentose phosphate pathway is that it regenerates ribulose-5-phosphate for the RuMP cycle and relies on RuMP pathway (Hps and Phi activity). Synthetic methylotrophy can be facilitated by balancing formaldehyde dissimilation and assimilation via the RuMP cycle. This can be achieved by deletion or attenuation of the linear pathway of formaldehyde dissimilation encoded by the endogenous genes frmR, frmA, frmB in E. coli or the introduction of an exogenous formaldehyde dissimilation pathway encoded by folD, fhs, and fdhA from B. methanolicus.

Alternatively, modifications can include the attenuation or deletion of endogenous zwf gene in E. coli. The endogenous zwf in E. coli is inhibited by high NADH levels and high pool sizes of F6P. Inhibition can be addressed by replacing the endogenous zwf of E. coli with an exogenous zwf2 that is non-naturally regulated and responsive to formaldehyde concentrations. An exemplary exogenous gene candidate is the zwf2 from B. methanolicus PB1 and MGA3. The engineered microorganism can express a tkt sequence that is related to Bacillus methanolicus PB1 or MGA3 zwf2, such as a Bacillus methanolicus PB1 or MGA3 zwf2 homolog or variant. For example, the zwf sequence can be 50% or greater, 60% or greater, 70% or greater, 80% or greater, 85% or greater, 90% or greater, or 95% or greater identity to Bacillus methanolicus PB1 or MGA3 zwf2 (SEQ ID NO:10).

Optionally, the engineered microorganism can include a modification that can attenuate or eliminate the activity of an endogenous zwf and introduce the activity of an exogenous zwf2 from B. methanolicus. This can also be combined with the introduction of gapN and the attenuation or deletion of endogenous GapA activity.

TABLE 16

| | | |
|---|---|---|
| zwf2 | Bacillus methanolicus MGA3 | fba2 |
| zwf2 | Bacillus methanolicus PB1 | WP_003350053.1 |

Candidate exogenous genes for introduction to balance formaldehyde dissimilation and assimilation include those in Table 17.

TABLE 17

| Gene | Organism | NCBI accession number |
|---|---|---|
| fdhA | Bacillus methanolicus MGA3 | WP_004434290.1 |
| fdhD | Bacillus methanolicus MGA3 | WP_004434293.1 |
| fhs | Bacillus methanolicus MGA3 | WP_004435057.1 |
| folD | Bacillus methanolicus MGA3 | WP_004435562.1 |

Acetate kinase catalyzes the formation of acetyl phosphate from acetate and ATP. The E. coli acetate kinase is encoded by ackA (Skarstedt and Silverstein, J. Biol. Chem. 251:6775-6783 (1976)), and phosphorylates propionate in addition to acetate (Hesslinger et al., wMol. Microbiol. 27:477-492 (1998)).Optionally, the engineered microorganism can include a modification that can attenuate or eliminate the activity of an acetate kinase. For example, the E. coli acetate ackA (Uniprot P0AC53) can be deleted or its activity attenuated. Deletion or attenuation of ackA can be beneficial in various strain embodiments, such as when phosphoketolase and/or and phosphotransacetylase are overexpressed. Since phosphoketolase forms acetyl_phosphate which can be converted to acetate, deletion of ackA and overexpression phosphotransacetylase can promote conversion of the resulting acetyl_phosphate to acetyl CoA.

In an embodiment, the engineered microorganism includes the following modifications: (1) exogenous enzyme A that (ai) is capable of converting glyceraldehyde 3-phosphate (G3P) to 3-phosphoglycerate (3PG), or (aii) has at least 50% sequence identity to SEQ ID NO:1 (B. methanolicus gapN), wherein enzyme A is capable of reducing NADP to NADPH, (2) exogenous hexulose-6-phosphate synthase (hps), (3) exogenous 6-phospho-3-hexuloisomeras (phi), (4) exogenous methanol dehydrogenase (MeDH), and (5) exogenous fructose-1,6-bisphosphatase (glpX).

In an embodiment, the engineered microorganism includes the following modifications: (1) exogenous enzyme A (e.g., gapN), (2) exogenous hps, (3) exogenous phi, (4) exogenous MeDH, (5) exogenous glpX, and (6) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, and (6) exogenous PK, and (7) endogenous methyl glyoxal synthase (mgsA) deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, and (6) endogenous NAD-dependent glyceraldehyde-3-phosphate dehydrogenase (gapA) deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous transaldolase activity (talB, talA, and/or talC) deletion or attenuation, (8) exogenous ATP-dependent 6-phosphofructokinase (pfk2), and (9) exogenous fructose-bisphosphate aldolase (fba).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), and (11) exogenous fructose-bisphosphate aldolase (fba).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), and (16) endogenous zwf deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, and (7) endogenous methyl glyoxal synthase (mgsA) deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, and (8) endogenous mgsA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), and (12) endogenous mgsA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, and (17) endogenous mgsA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, and (20) endogenous mgsA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous mgsA deletion or attenuation, and (8) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, (8) endogenous mgsA deletion or attenuation, (9) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous transaldolase activity (talB, talA, and/or talC) deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, and (13) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, and (18) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gapA deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, and (21) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous phosphoglycerate kinase (pgk) deletion or attenuation, (7) endogenous mgsA deletion or attenuation, and (8) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous pgk deletion or attenuation, (7) endogenous talB, talA, and/or talC deletion or attenuation, (7) endogenous mgsA deletion or attenuation, (8) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous pgk deletion or attenuation, (7) endogenous transaldolase activity (talB, talA, and/or talC) deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, and (13) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous pgk deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, and (18) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous phosphoglycerate kinase (pgk) deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, and (21) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous phosphoglycerate mutase (gpm) deletion or attenuation, (7) endogenous mgsA deletion or attenuation, and (8) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gpm deletion or attenuation, (7) endogenous talB, talA, and/or talC deletion or attenuation, (8) endogenous mgsA deletion or attenuation, (9) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gpm deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, and (13) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gpm deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, and (18) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous gpm deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, and (21) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous enolase (eno) deletion or attenuation, (7) endogenous mgsA deletion or attenuation, and (8) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous eno deletion or attenuation, (7) endogenous talB, talA, and/or talC deletion or attenuation, (8) endogenous mgsA deletion or attenuation, (9) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous eno deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, and (13) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous eno deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, and (18) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous eno deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, and (21) exogenous phosphoketolase (PK).

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoxyribose phosphate aldolase (deoC) deletion or attenuation, (7) endogenous mgsA deletion or attenuation, (8) exogenous phosphoketolase (PK), and (9) endogenous gapA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) endogenous mgsA deletion or attenuation, (9) exogenous phosphoketolase (PK), and (10) endogenous gapA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, (13) exogenous phosphoketolase (PK), and (14) endogenous gapA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, (18) exogenous phosphoketolase (PK), and (19) endogenous gapA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, (21) exogenous phosphoketolase (PK), and (22) endogenous gapA deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoxyribose phosphate aldolase (deoC) deletion or attenuation, (7) endogenous mgsA deletion or attenuation, (8) exogenous phosphoketolase (PK), and (9) endogenous pgk deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) endogenous mgsA deletion or attenuation, (9) exogenous phosphoketolase (PK), and (10) endogenous pgk deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, (13) exogenous phosphoketolase (PK), and (14) endogenous pgk deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, (18) exogenous phosphoketolase (PK), and (19) endogenous pgk deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, (21) exogenous phosphoketolase (PK), and (22) endogenous pgk deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoxyribose phosphate aldolase (deoC) deletion or attenuation, (7) endogenous mgsA deletion or attenuation, (8) exogenous phosphoketolase (PK), and (9) endogenous gpm deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) endogenous mgsA deletion or attenuation, (9) exogenous phosphoketolase (PK), and (10) endogenous gpm deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) exogenous ribulose-phosphate 3-epimerase (rpe), (9) exogenous ribose-5-phosphate isomerase (rpi), (10) exogenous transketolase (tkt), (11) exogenous fructose-bisphosphate aldolase (fba), (12) endogenous mgsA deletion or attenuation, (13) exogenous phosphoketolase (PK), and (14) endogenous gpm deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) exogenous ribulose-phosphate 3-epimerase (rpe), (8) endogenous rpe deletion or attenuation, (9) exogenous ribose-5-phosphate isomerase (rpi), (10) endogenous rpi deletion or attenuation, (11) endogenous transketolase (tkt) deletion or attenuation, (12) exogenous transketolase (tkt), (13) exogenous fructose-bisphosphate aldolase (fba), (14) endogenous fba deletion or attenuation, (15) exogenous glucose-6-phosphate 1-dehydrogenase (zwf), (16) endogenous zwf deletion or attenuation, (17) endogenous mgsA deletion or attenuation, (18) exogenous phosphoketolase (PK), and (19) endogenous gpm deletion or attenuation.

In an embodiment, the engineered microorganism includes the following modifications: (1) exogenous enzyme A (e.g., gapN), (2) exogenous hps, (3) exogenous phi, (4) exogenous MeDH, (5) exogenous glpX, (6) endogenous deoC deletion or attenuation, (7) endogenous talB, talA, and/or talC deletion or attenuation, (8) exogenous ATP-dependent 6-phosphofructokinase (pfk), (9) endogenous pfk deletion or attenuation, (10) exogenous ribulose-phosphate 3-epimerase (rpe), (11) endogenous rpe deletion or attenuation, (12) exogenous ribose-5-phosphate isomerase (rpi), (13) endogenous rpi deletion or attenuation, (14) endogenous transketolase (tkt) deletion or attenuation, (15) exogenous transketolase (tkt), (16) exogenous fructose-bisphosphate aldolase (fba), (17) endogenous fba deletion or attenuation, (18) exogenous glucose-6-phosphate 1-dehydrogenase (zwf), and (19) endogenous zwf deletion or attenuation, (20) endogenous mgsA deletion or attenuation, (21) exogenous phosphoketolase (PK), and (22) endogenous gpm deletion or attenuation. In an embodiment, the engineered microorganism includes the following modifications: (1) exogenous hps, (2) exogenous phi, (3) exogenous MeDH, (4) exogenous glpX, (5) endogenous transaldolase activity (talB, talA, and/or talC) deletion or attenuation, (6) exogenous ATP-dependent 6-phosphofructokinase (pfk2), and (7) exogenous fructose-bisphosphate aldolase (fba).

In an embodiment, the engineered microorganism has the following modifications: (1) exogenous hps, (2) exogenous phi, (3) exogenous MeDH, (4) exogenous glpX, (5) endogenous talB, talA, and/or talC deletion or attenuation, (6) exogenous ribulose-phosphate 3-epimerase (rpe), (7) exogenous ribose-5-phosphate isomerase (rpi), (8) exogenous transketolase (tkt), and (9) exogenous fructose-bisphosphate aldolase (fba).

In an embodiment, the engineered microorganism includes the following modifications: (1) exogenous hps, (2) exogenous phi, (3) exogenous MeDH, (4) exogenous glpX, (5) endogenous talB, talA, and/or talC deletion or attenuation, (6) exogenous ATP-dependent 6-phosphofructokinase (pfk), (7) endogenous pfk deletion or attenuation, (8) exogenous ribulose-phosphate 3-epimerase (rpe), (9) endogenous rpe deletion or attenuation, (10) exogenous ribose-5-phosphate isomerase (rpi), (11) endogenous rpi deletion or attenuation, (12) endogenous transketolase (tkt) deletion or attenuation, (13) exogenous transketolase (tkt), (14) exogenous fructose-bisphosphate aldolase (fba), (15) endogenous fba deletion or attenuation, (16) exogenous glucose-6-phosphate 1-dehydrogenase (zwf), and (17) endogenous zwf deletion or attenuation.

In an embodiment, the engineered microorganism includes the following modifications: (1) exogenous hps, (2) exogenous phi, (3) exogenous MeDH, (4) exogenous glpX, (5) endogenous talB, talA, and/or talC deletion or attenuation, (6) exogenous ATP-dependent 6-phosphofructokinase (pfk), (7) endogenous pfk deletion or attenuation, (8) exogenous ribulose-phosphate 3-epimerase (rpe), (9) endogenous rpe deletion or attenuation, (10) exogenous ribose-5-phosphate isomerase (rpi), (11) endogenous rpi deletion or attenuation, (12) endogenous transketolase (tkt) deletion or attenuation, (13) exogenous transketolase (tkt), (14) exogenous fructose-bisphosphate aldolase (fba), (15) endogenous fba deletion or attenuation, (16) exogenous glucose-6-phosphate 1-dehydrogenase (zwf), and (17) endogenous zwf deletion or attenuation, (18) endogenous mgsA deletion or attenuation, and (19) exogenous phosphoketolase (PK).

In an embodiment, the engineered microorganism includes the following modifications: (1) exogenous hps, (2) exogenous phi, (3) exogenous MeDH, (4) exogenous glpX, (5) endogenous talB, talA, and/or talC deletion or attenuation, (6) endogenous mgsA deletion or attenuation, (7) exogenous phosphoketolase (PK).

In an embodiment, the engineered microorganism includes the following modifications: (1) exogenous hps, (2) exogenous phi, (3) exogenous MeDH, (4) exogenous glpX, (5) endogenous transaldolase activity (talB, talA, and/or talC) deletion or attenuation, (6) exogenous ribulose-phosphate 3-epimerase (rpe), (7) exogenous ribose-5-phosphate isomerase (rpi), (8) exogenous transketolase (tkt), (9) exogenous fructose-bisphosphate aldolase (fba), (10) endogenous mgsA deletion or attenuation, and (11) exogenous phosphoketolase (PK).

In an embodiment, the engineered microorganism has the following modifications: (1) exogenous enzyme A (e.g., gapN), (2) exogenous hps, (3) exogenous phi, (4) exogenous MeDH, (5) exogenous glpX, (6) endogenous talB, talA, and/or talC deletion or attenuation, (7) exogenous ATP-dependent 6-phosphofructokinase (pfkA and pfkB), (8) exogenous ribulose-phosphate 3-epimerase (rpe), (9) endogenous rpe deletion or attenuation, (10) exogenous ribose-5-phosphate isomerase (rpi), (11) endogenous rpi deletion or attenuation, (12) endogenous transketolase (tkt) deletion or attenuation, (13) exogenous transketolase (tkt), (14) exogenous fructose-bisphosphate aldolase (fba), (15) endogenous fba deletion or attenuation, (15) exogenous glucose-6-phosphate 1-dehydrogenase (zwf), and (16) endogenous zwf deletion or attenuation, (17) endogenous mgsA deletion or attenuation, (18) exogenous phosphoketolase (PK).

Given the teachings and guidance provided herein, those skilled in the art also will understand that enzymatic activity or expression can be attenuated using well known methods. Reduction of the activity or amount of an enzyme can mimic complete disruption of a gene if the reduction causes activity of the enzyme to fall below a critical level that is normally required for a pathway to function. Reduction of enzymatic activity by various techniques rather than use of a gene disruption can be important for an organism's viability. Methods of reducing enzymatic activity that result in similar or identical effects of a gene disruption include, but are not limited to: reducing gene transcription or translation; destabilizing mRNA, protein or catalytic RNA; and mutating a gene that affects enzyme activity or kinetics (See, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999). Natural or imposed regulatory controls can also accomplish enzyme attenuation including: promoter replacement (See, Wang et al., Mol. Biotechnol. 52(2):300-308 (2012)); loss or alteration of transcription factors (Dietrick et al., Annu. Rev. Biochem. 79:563-590 (2010); and Simicevic et al., Mol. Biosyst. 6(3):462-468 (2010)); introduction of inhibitory RNAs or peptides such as siRNA, antisense RNA, RNA or peptide/small-molecule binding aptamers, ribozymes, aptazymes and riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); and addition of drugs or other chemicals that reduce or disrupt enzymatic activity such as an enzyme inhibitor, an antibiotic or a target-specific drug.

One skilled in the art will also understand and recognize that attenuation of an enzyme can be done at various levels. For example, at the gene level, a mutation causing a partial or complete null phenotype, such as a gene disruption, or a mutation causing epistatic genetic effects that mask the activity of a gene product (Miko, Nature Education 1(1) (2008)), can be used to attenuate an enzyme. At the gene expression level, methods for attenuation include: coupling transcription to an endogenous or exogenous inducer, such as isopropylthio-β-galactoside (IPTG), then adding low amounts of inducer or no inducer during the production phase (Donovan et al., J. Ind. Microbiol. 16(3):145-154 (1996); and Hansen et al., Curr. Microbiol. 36(6):341-347 (1998)); introducing or modifying a positive or a negative regulator of a gene; modify histone acetylation/deacetylation in a eukaryotic chromosomal region where a gene is integrated (Yang et al., Curr. Opin. Genet. Dev. 13(2):143-153 (2003) and Kurdistani et al., Nat. Rev. Mol. Cell Biol. 4(4):276-284 (2003)); introducing a transposition to disrupt a promoter or a regulatory gene (Bleykasten-Brosshans et al., C. R. Biol. 33(8-9):679-686 (2011); and McCue et al., PLoS Genet. 8(2):e1002474 (2012)); flipping the orientation of a transposable element or promoter region so as to modulate gene expression of an adjacent gene (Wang et al., Genetics 120(4):875-885 (1988); Hayes, Annu. Rev. Genet. 37:3-29 (2003); in a diploid organism, deleting one allele resulting in loss of heterozygosity (Daigaku et al., Mutation Research/Fundamental and Molecular Mechanisms of Mutagenesis 600(1-2)177-183 (2006)); introducing nucleic acids that increase RNA degradation (Houseley et al., Cell, 136(4):763-776 (2009); or in bacteria, for example, introduction of a transfer-messenger RNA (tmRNA) tag, which can lead to RNA degradation and ribosomal stalling (Sunohara et al., RNA 10(3):378-386 (2004); and Sunohara et al., J. Biol. Chem. 279:15368-15375 (2004)). At the translational level, attenuation can include: introducing rare codons to limit translation (Angov, Biotechnol. J. 6(6):650-659 (2011)); introducing RNA interference molecules that block translation (Castel et al., Nat. Rev. Genet. 14(2):100-112(2013); and Kawasaki et al., Curr. Opin. Mol. Ther. 7(2):125-131 (2005); modifying regions outside the coding sequence, such as introducing secondary structure into an untranslated region (UTR) to block translation or reduce efficiency of translation (Ringnr et al., PLoS Comput. Biol. 1(7):e72 (2005)); adding RNAase sites for rapid transcript degradation (Pasquinelli, Nat. Rev. Genet. 13(4):271-282 (2012); and Arraiano et al., FEMS Microbiol. Rev. 34(5):883-932 (2010); introducing antisense RNA oligomers or antisense transcripts (Nashizawa et al., Front. Biosci. 17:938-958 (2012)); introducing RNA or peptide aptamers, ribozymes, aptazymes, riboswitches (Wieland et al., Methods 56(3):351-357 (2012); O'Sullivan, Anal. Bioanal. Chem. 372(1):44-48 (2002); and Lee et al., Curr. Opin. Biotechnol. 14(5):505-511 (2003)); or introducing translational regulatory elements involving RNA structure that can prevent or reduce translation that can be controlled by the presence or absence of small molecules (Araujo et al., Comparative and Functional Genomics, Article ID 475731, 8 pages (2012)). At the level of enzyme localization and/or longevity, enzyme attenuation can include: adding a degradation tag for faster protein turnover (Hochstrasser, Annual Rev. Genet. 30:405-439 (1996); and Yuan et al., PLoS One 8(4):e62529 (2013)); or adding a localization tag that results in the enzyme being secreted or localized to a subcellular compartment in a eukaryotic cell, where the enzyme would not be able to react with its normal substrate (Nakai et al. Genomics 14(4):897-911 (1992); and Russell et al., J. Bact. 189(21)7581-7585 (2007)). At the level of post-translational regulation, enzyme attenuation can include: increasing intracellular concentration of known inhibitors; or modifying post-translational modified sites (Mann et al., Nature Biotech. 21:255-261 (2003)). At the level of enzyme activity, enzyme attenuation can include: adding an endogenous or an exogenous inhibitor, such as an enzyme inhibitor, an antibiotic or a target-specific drug, to reduce enzyme activity; chelating a metal ion that is required for enzyme activity; or introducing a dominant negative mutation. The applicability of a technique for attenuation described above can depend upon whether a given host microbial organism is prokaryotic or eukaryotic, and it is understand that a determination of what is the appropriate technique for a given host can be readily made by one skilled in the art.

An expression vector or vectors can be constructed to include one or more exogenous protein-encoding nucleic acids as exemplified herein, operably linked to expression control sequences functional in the host organism. Expression vectors applicable for use in the microbial host organisms provided include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art.

The exogenous protein(s) can be co-expressed with one or more additional nucleic acids that may encode enzyme(s) useful for converting intermediates. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism, the more than one exogenous nucleic acid refers to the referenced encoding nucleic acid or biosynthetic activity, as discussed above. It is further understood, as disclosed herein, that more than one exogenous nucleic acid can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein, such as an exogenous nucleic acid that expresses an exogenous protein of the disclosure, and one or more other enzymes that convert an intermediate generated from the methylotrophic pathway to a desired bioproduct.

In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

Exogenous enzyme-encoding nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As discussed herein, a microbial organism can be described in terms of its inability or ability to utilize C1 compounds such as methanol as a source of energy and cellular carbon. A host cell in which exogenous enzymes are introduced to generate synthetic methylotrophy can be described as a “non-methylotrophic,” that is, the host cell without engineering is unable to utilize a 1C carbon as an energy source. Microorganisms that are non-methylotrophic and that can be engineered with exogenous enzyme(s) of the disclosure include E. coli and other prokaryotic and eukaryotic organisms as described herein.

The term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments.

In some aspects a nucleic acid encoding an exogenous enzyme of the disclosure is introduced into a cell with a gene disruption. The term “gene disruption,” or grammatical equivalents thereof, is intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. One particularly useful method of gene disruption is complete gene deletion because it reduces or eliminates the occurrence of genetic reversions. The phenotypic effect of a gene disruption can be a null mutation, which can arise from many types of mutations including inactivating point mutations, entire gene deletions, and deletions of chromosomal segments or entire chromosomes. Specific antisense nucleic acid compounds and enzyme inhibitors, such as antibiotics, can also produce null mutant phenotype, therefore being equivalent to gene disruption.

A metabolic modification refers to a biochemical reaction that is altered from its naturally occurring state. Therefore, microorganisms may have genetic modifications to nucleic acids encoding metabolic polypeptides, or functional fragments thereof. Exemplary metabolic modifications are disclosed herein.

The microorganisms provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

A variety of microorganism may be suitable for incorporating one or more nucleic acid encoding protein(s) which allow the cell to have synthetic or enhanced methylotrophy. Such organisms include both prokaryotic and eukaryotic organisms including, but not limited to, bacteria, including archaea and eubacteria, and eukaryotes, including yeast, plant, insect, animal, and mammal, including human. Exemplary species are reported in U.S. Patent Pub No. 2014/0058056 (Burgard et al.), which is incorporated herein by reference, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium dificile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrifcans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In certain embodiments, suitable organisms include Acinetobacter baumannii Naval-82, Acinetobacter sp. ADP1, Acinetobacter sp. strain M-1, Actinobacillus succinogenes 130Z Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB), Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium dificile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfltobacterium hafniense, Desulfltobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM), Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. L72, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Tsukamurella paurometabola DSM 20162, Vibrio harveyi ATCC BAA-1116, Xanthobacter autotrophicus Py2, and Yersinia intermedia.

Therefore, an engineered cell having synthetic or enhanced methylotrophy including (a) exogenous enzyme A that (ai) is capable of converting glyceraldehyde 3-phosphate (G3P) to 3-phosphoglycerate (3PG), that (aii) has at least 50% sequence identity to SEQ ID NO:1 (B. methanolicus gapN), wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), can include one or more further genetic alterations, such as inserted transgenes, deletions, attenuation, mutations, etc., desired to increase levels of one or more intermediates or a product thereof, and include those genetic modifications as described in U.S. Patent Pub No. 2014/0058056 (Burgard et at), and include those genetic modifications as described in WO2009/135074 (Burk et al.), the disclosures of which are incorporated herein by reference.

Of particular interest are target products obtained using pyruvate and acetyl-CoA as entry point or precursor to its product pathway(s), in part because the methanol metabolic pathway using the novel enzymes enables fixing the carbon of methanol into pathways to pyruvate and acetyl-CoA. Target products include (a) 1,4-butanediol and intermediates thereto, such as 4-hydroxybutanoic acid (4-hydroxybutanoate, 4-hydroxybutyrate, 4-HB), (b) butadiene and intermediates thereto, such as 1,4-butanediol, 1,3-butanediol, crotyl alcohol, 3-buten-2-ol (methyl vinyl carbinol) and 3-buten-1-ol, (c) 1,3-butanediol and intermediates thereto, such as 2,4-pentadienoate, crotyl alcohol or 3-buten-1-ol, (d) adipate, 6-aminocaproic acid, caprolactam, hexamethylenediamine and levulinic acid and their intermediates, e.g. 4-aminobutyryl-CoA, (e) methacrylic acid (2-methyl-2-propenoic acid) and its esters known collectively as methacrylates, such as methyl methacrylate, methyl methacrylate, 3-hydroxyisobutyrate and/or 2-hydroxyisobutyrate and their intermediates, (f) 1,2-propanediol (propylene glycol), n-propanol, 1,3-propanediol and glycerol, and their intermediates and (g) succinic acid and intermediates thereto.

In some aspects, an engineered cell having synthetic or enhanced methylotrophy including (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), is used with a product pathway for increased levels of 1,4-butanediol (BDO) or hydroxylbutyrate (4-HB). Those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

With the complete genome sequence available for now more than 550 species (with more than half of these available on public databases such as the NCBI), including 395 microorganism genomes and a variety of yeast, fungi, plant, and mammalian genomes, the identification of genes encoding the requisite BDO or 4-HB biosynthetic pathway as well as other known biosynthetic pathways for 1,3-butanediol (13BDO), butadiene, 6-amino caproic acid (6ACA), hexamethylenediamine (HMDA), adipic acid or derivatives thereof, croytl alcohol, methyl vinyl carbinol, 3-buten-1-ol, succinic acid or derivatives thereof, n-propanol, isopropanol, propylene, methacrylic acid or derivatives thereof, methanol metabolic and/or formaldehyde assimilation activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of various target products including 1,3-butanediol (13BDO), 1, 4-butanediol (BDO), 4-HB, butadiene, 6-amino caproic acid (6ACA), hexamethyldiamine (HMDA), adipic acid or derivatives thereof, croytl alcohol, methyl vinyl carbinol, 3-buten-1-ol, succinic acid or derivatives thereof, n-propanol, isopropanol, propylene, methacrylic acid or derivatives thereof, metabolism of methanol and/or assimilation of formaldehyde described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

Exemplary alcohol metabolic pathway gene(s), such as described in U.S. Patent Pub No. 2014/0058056, encode a protein selected from the group consisting of: a formate dehydrogenase, a formaldehyde activating enzyme, a formaldehyde dehydrogenase, a S-(hydroxymethyl)glutathione synthase, a glutathione-dependent formaldehyde dehydrogenase, a S-formylglutathione hydrolase, a formate hydrogen lyase, and a hydrogenase, any or more can be coexpressed with (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), in the engineered cell.

Other exemplary alcohol metabolic pathway gene(s), such as described in U.S. Patent Pub No. 2014/0058056, encode an alcohol metabolic pathway gene(s) encoding a protein selected from the group consisting of a succinyl-CoA reductase (aldehyde forming), a 4-hydroxybutyrate (4-HB) dehydrogenase, a 4-HB kinase, a phosphotrans-4-hydroxybutyrylase, a 4-hydroxybutyryl-CoA reductase (aldehyde forming), a 1,4-butanediol dehydrogenase; a succinate reductase, a succinyl-CoA reductase (alcohol forming), 4-hydroxybutyryl-CoA transferase, a 4-hydroxybutyryl-CoA synthetase, a 4-HB reductase, and a 4-hydroxybutyryl-CoA reductase (alcohol forming), a succinyl-CoA transferase, and a succinyl-CoA synthetase, any or more can be co-expressed with (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), in the engineered cell.

1,4-butanediol and intermediates thereto, such as 4-hydroxybutanoic acid (4-hydroxybutanoate, 4-hydroxybutyrate, 4-HB), are target products that can be made by co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described herein as well as in the following documents. Suitable product pathways and enzymes, methods for screening and methods for isolating are found herein as well as in the following documents, incorporated herein by reference: WO2008115840A2 published 25 Sep. 2008 entitled “Compositions and Methods for the Biosynthesis of 1, 4-Butanediol and Its Precursors”; WO2010141780A1 published 9 Dec. 2010 entitled “Process of Separating Components of A Fermentation Broth”; WO2010141920A2 published 9 Dec. 2010 entitled “Microorganisms for the Production of 1, 4-Butanediol and Related Methods”; WO2010030711A2 published 18 Mar. 2010 entitled “Microorganisms for the Production of 1, 4-Butanediol”; WO2010071697A1 published 24 Jun. 2010 entitled “Microorganisms and Methods for Conversion of Syngas and Other Carbon Sources to Useful Products”; WO2009094485A1 published 30 Jul. 2009 entitled “Methods and Organisms for Utilizing Synthesis Gas or Other Gaseous Carbon Sources and Methanol”; WO2009023493A1 published 19 Feb. 2009 entitled “Methods and Organisms for the Growth-Coupled Production of 1,4-Butanediol”; WO2008115840A2 published 25 Sep. 2008 entitled “Compositions and Methods for the Biosynthesis of 1,4-Butanediol and Its Precursors”; and International Application No. PCT/US13/56725 filed 27 Aug. 2013 entitled “Microorganisms an Methods for Enhancing the Availability of Reducing Equivalents in the Presence of Methanol, and for Producing 1,4-Butanediol Related Thereto”.

Butadiene and intermediates thereto, such as 1,4-butanediol, 1,3-butanediol, crotyl alcohol, 3-buten-2-ol (methyl vinyl carbinol) and 3-buten-1-ol, are target products that can be made by co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described in the following documents. In addition to direct fermentation to produce butadiene, 1,3-butanediol, 1,4-butanediol, crotyl alcohol, 3-buten-2-ol (methyl vinyl carbinol) and 3-buten-1-ol can be separated, purified (for any use), and then dehydrated to butadiene in a second step involving metal-based catalysis. Suitable product pathways and enzymes, methods for screening and methods for isolating are found in the following documents, incorporated herein by reference: WO2011140171A2 published 10 Nov. 2011 entitled “Microorganisms and Methods for the Biosynthesis of Butadiene”; WO2012018624A2 published 9 Feb. 2012 entitled “Microorganisms and Methods for the Biosynthesis of Aromatics, 2,4-Pentadienoate and 1,3-Butadiene”; WO2011140171A2 published 10 Nov. 2011 entitled “Microorganisms and Methods for the Biosynthesis of Butadiene”; WO2013040383A1 published 21 Mar. 2013 entitled “Microorganisms and Methods for Producing Alkenes”; WO2012177710A1 published 27 Dec. 2012 entitled “Microorganisms for Producing Butadiene and Methods Related thereto”; WO2012106516A1 published 9 Aug. 2012 entitled “Microorganisms and Methods for the Biosynthesis of Butadiene”; WO2013028519A1 published 28 Feb. 2013 entitled “Microorganisms and Methods for Producing 2,4-Pentadienoate, Butadiene, Propylene, 1,3-Butanediol and Related Alcohols”; and U.S. Patent Pub No. 2015/0050708. 1,3-butanediol and intermediates thereto, such as 2,4-pentadienoate, crotyl alcohol or 3-buten-1-ol, are target products that can be made by co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described herein as well as in the following documents. Suitable product pathways and enzymes, methods for screening and methods for isolating are found herein as well as in the following documents, incorporated herein by reference: WO2011071682A1 published 16 Jun. 2011 entitled “Methods and Organisms for Converting Synthesis Gas or Other Gaseous Carbon Sources and Methanol to 1, 3-Butanediol”; WO2011031897A published 17 Mar. 2011 entitled “Microorganisms and Methods for the Co-Production of Isopropanol with Primary Alcohols, Diols and Acids”; WO2010127319A2 published 4 Nov. 2010 entitled “Organisms for the Production of 1,3-Butanediol”; WO2013071226A1 published 16 May 2013 entitled “Eukaryotic Organisms and Methods for Increasing the Availability of Cytosolic Acetyl-CoA, and for Producing 1,3-Butanediol”; WO2013028519A1 published 28 Feb. 2013 entitled “Microorganisms and Methods for Producing 2,4-Pentadienoate, Butadiene, Propylene, 1,3-Butanediol and Related Alcohols”; WO2013036764A1 published 14 Mar. 2013 entitled “Eukaryotic Organisms and Methods for Producing 1,3-Butanediol”; WO2013012975A1 published 24 Jan. 2013 entitled “Methods for Increasing Product Yields”; WO2012177619A2 published 27 Dec. 2012 entitled “Microorganisms for Producing 1, 3-Butanediol and Methods Related Thereto”; and U.S. Patent Pub No. 2015/0050708.

Adipate, 6-aminocaproic acid, caprolactam, hexamethylenediamine and levulinic acid, and their intermediates, e.g. 4-aminobutyryl-CoA, are target products, useful for example for making nylon polymers, that can be made by co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described herein as well as in the following documents. Suitable product pathways and enzymes, methods for screening and methods for isolating are found herein as well as in the following documents, incorporated herein by reference: WO2010129936A1 published 11 Nov. 2010 entitled “Microorganisms and Methods for the Biosynthesis of Adipate, Hexamethylenediamine and 6-Aminocaproic Acid”; WO2013012975A1 published 24 Jan. 2013 entitled “Methods for Increasing Product Yields”; WO2012177721A1 published 27 Dec. 2012 entitled “Microorganisms for Producing 6-Aminocaproic Acid”; WO2012099621A1 published 26 Jul. 2012 entitled “Methods for Increasing Product Yields”; and U.S. Patent Pub No. 2014/0329916 entitled “Microorganisms an Methods for Enhancing the Availability of Reducing Equivalents in the Presence of Methanol, and for Producing Adipate, 6-Aminocaproate, Hexamethylenediamine or Caprolactam Related Thereto”.

Methacrylic acid (2-methyl-2-propenoic acid; used in the preparation of its esters known collectively as methacrylates, such as methyl methacrylate, which is used most notably in the manufacture of polymers), methacrylate ester such as methyl methacrylate, 3-hydroxyisobutyrate and/or 2-hydroxyisobutyrate and their intermediates are target products, useful for example for making polymers, that can be made by co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described herein as well as in the following documents. Suitable product pathways and enzymes, methods for screening and methods for isolating are found herein as well as in the following documents, incorporated herein by reference: WO2012135789A2 published 4 Oct. 2012 entitled “Microorganisms for Producing Methacrylic Acid and Methacrylate Esters and Methods Related Thereto”; WO2009135074A2 published 5 Nov. 2009 entitled “Microorganisms for the Production of Methacrylic Acid”; and U.S. Patent Pub No. 2014/0288254 entitled “Microorganisms an Methods for Enhancing the Availability of Reducing Equivalents in the Presence of Methanol, and for Producing 3-Hydroxyisobutyate or Methacrylic Acid Related Thereto”.

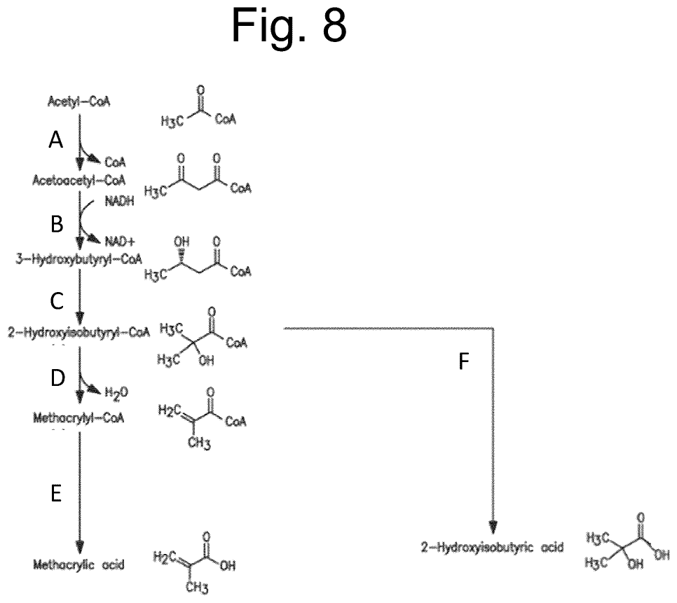

FIG. 8 illustrates exemplary target product pathways, 2-hydroxyisobutyrate and methacrylic acid from acetyl-CoA/methyl-methacrylate (MMA), which can exploit acetyl-CoA available from methanol assimilation as disclosed herein, 2-Hydroxyisobutyrate and methacrylic acid production can be carried out by the following enzymes: A) acetyl-CoA:acetyl-CoA acyltransferase, B) acetoacetyl-CoA reductase (ketone reducing), C) 3-hydroxybutyrl-CoA mutase, D) 2-hydroxyisobutyryl-CoA dehydratase, E) methacrylyl-CoA synthetase, hydrolase, or transferase, F) 2-hydroxyisobutyryl-CoA synthetase, hydrolase, or transferase.

1,2-propanediol (propylene glycol), n-propanol, 1,3-propanediol and glycerol, and their intermediates are target products, useful for example for making polymers, that can be made by co-expressing the (a) exogenous enzyme A that (ai) is capable of converting G3P to 3PG. that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described herein as well as in the following documents. Suitable product pathways and enzymes, methods for screening and methods for isolating are found herein as well as in the following documents, incorporated herein by reference: WO2009111672A1 published 9 Nov. 2009 entitled “Primary Alcohol Producing Organisms”; WO2011031897A1 17 Mar. 2011 entitled “Microorganisms and Methods for the Co-Production of Isopropanol with Primary Alcohols, Diols and Acids”; WO2012177599A2 published 27 Dec. 2012 entitled “Microorganisms for Producing N-Propanol 1, 3-Propanediol, 1, 2-Propanediol or Glycerol and Methods Related Thereto”; and U.S. Patent Pub No. 2014/0302575 entitled “Microorganisms an Methods for Enhancing the Availability of Reducing Equivalents in the Presence of Methanol, and for Producing 1,2-Propanediol, n-Propanol, 1,3-Propanediol, or Glycerol Related Thereto”.

Succinic acid and intermediates thereto (useful to produce products including polymers, e.g. PBS, 1,4-butanediol, tetrahydrofuran, pyrrolidone, solvents, paints, deicers, plastics, fuel additives, fabrics, carpets, pigments, and detergents) are target products that can be made by co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein with a product pathway described herein as well as in the following documents. Suitable product pathways and enzymes, methods for screening and methods for isolating are found herein as well as in the following documents, incorporated herein by reference: EP1937821A2 published 2 Jul. 2008 entitled “Methods and Organisms for the Growth-Coupled Production of Succinate”; and U.S. Patent Pub No. 2014/0302575 entitled “Microorganisms and Methods for Enhancing the Availability of Reducing Equivalents in the Presence of Methanol, and for Producing Succinate Related Thereto”.

Target products obtained from, and product pathways suitable for producing in, host cells co-expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), described herein include the following. Butadiene and intermediates thereto, such as 1,4-butanediol, 1,3-butanediol, crotyl alcohol, 3-buten-2-ol (methyl vinyl carbinol) and 3-buten-1-ol, are target products that can be made by co-expressing the expressing the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), with a product pathway described in the following documents. In addition to direct fermentation to produce butadiene, 1,3-butanediol, 1,4-butanediol, crotyl alcohol, 3-buten-2-ol (methyl vinyl carbinol) and 3-buten-1-ol can be separated, purified (for any use), and then dehydrated to butadiene in a second step involving metal-based catalysis. Suitable product pathways and enzymes, methods for screening and methods for isolating are found in: WO2011140171A2 published 10 Nov. 2011 entitled “Microorganisms and Methods for the Biosynthesis of Butadiene”; WO2012018624A2 published 9 Feb. 2012 entitled “Microorganisms and Methods for the Biosynthesis of Aromatics, 2,4-Pentadienoate and 1,3-Butadiene”; WO2011140171A2 published 10 Nov. 2011 entitled “Microorganisms and Methods for the Biosynthesis of Butadiene”; WO2013040383A1 published 21 Mar. 2013 entitled “Microorganisms and Methods for Producing Alkenes”; WO2012177710A1 published 27 Dec. 2012 entitled “Microorganisms for Producing Butadiene and Methods Related thereto”; WO2012106516A1 published 9 Aug. 2012 entitled “Microorganisms and Methods for the Biosynthesis of Butadiene”; WO2013028519A1 published 28 Feb. 2013 entitled “Microorganisms and Methods for Producing 2,4-Pentadienoate, Butadiene, Propylene, 1,3-Butanediol and Related Alcohols”; and U.S. Patent Pub No. 2015/0050708.

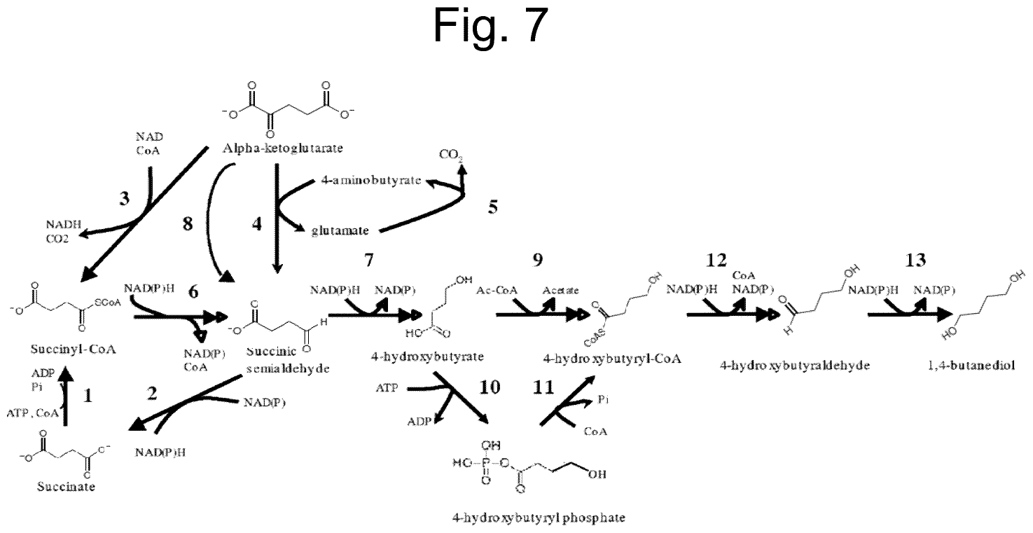

Enzymes, genes and methods for engineering pathways from acetyl-CoA, succinate and succinyl-CoA to various products, such as BDO, into a microorganism, are now known in the art (see, e.g., U.S. Publ. No. 2011/0201089). A set of BDO pathway enzymes represents a group of enzymes that can convert succinate or alpha-ketoglutarate to BDO as shown in FIG. 7. For example, BDO can be produced from succinyl-CoA via previously disclosed pathways (see for example, Burk et al., WO 2008/115840). FIG. 7 presents exemplary pathways which can use the primary metabolites, e.g. acetyl-CoA, made available by the use of methanol as a carbon source as described herein. In FIG. 7, the organism comprises at least one exogenous nucleic acid encoding a BDO pathway enzyme expressed in a sufficient amount to produce BDO. In certain embodiments, the BDO pathway enzyme is selected from the group consisting of (1) a succinyl-CoA synthetase; (2) a CoA-independent succinic semialdehyde dehydrogenase; (3) a α-ketoglutarate dehydrogenase; (4) a glutamate:succinate semialdehyde transaminase; (5) a glutamate decarboxylase; (6) a CoA-dependent succinic semialdehyde dehydrogenase; (7) a 4-hydroxybutanoate dehydrogenase; (8) a a-ketoglutarate decarboxylase; (9) a 4-hydroxybutyryl CoA:acetyl-CoA transferase; (10) a butyrate kinase; (11) a phosphotransbutyrylase; (12) an aldehyde dehydrogenase; and (13) an alcohol dehydrogenase. Preferred pathways include those from alpha-ketoglutarate, e.g. steps 8, 7, 9, 12 and 13; steps 3, 6, 7, 9, 12 and 13; and steps 1, 6, 7, 9, 12, 13. In an alternative, a single protein can comprise the activities of steps 12 and 13. Specific enzymes, classes of enzymes and sources of enzymes and their genes can be found in WO2008115840, for example.

In some embodiments, the disclosure provides organisms comprising (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), and that are engineered to improve the availability of reducing equivalents, which can be used for the production of target product molecules. It will be recognized by one skilled in the art that any product molecule that utilizes reducing equivalents in its production can exhibit enhanced production through other biosynthetic pathways.

In numerous engineered pathways, realization of maximum product yields based on carbohydrate feedstock is hampered by insufficient reducing equivalents or by loss of reducing equivalents to byproducts. Methanol is a relatively inexpensive organic feedstock that can be used to generate reducing equivalents by using the (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), and one or more metabolic enzymes. The reducing equivalents produced by the metabolism of methanol can then be used to power the glucose to BDO production pathways, for example, as shown in FIG. 7.

FIG. 7 presents exemplary pathways which can use the primary metabolites, e.g. acetyl-CoA, made available by the use of methanol as a carbon source as described herein. In FIG. 7, the organism comprises at least one exogenous nucleic acid encoding a BDO pathway enzyme expressed in a sufficient amount to produce BDO. In certain embodiments, the BDO pathway enzyme is selected from the group consisting of (1) a succinyl-CoA synthetase; (2) a CoA-independent succinic semialdehyde dehydrogenase; (3) a a-ketoglutarate dehydrogenase; (4) a glutamate:succinate semialdehyde transaminase; (5) a glutamate decarboxylase; (6) a CoA-dependent succinic semialdehyde dehydrogenase; (7) a 4-hydroxybutanoate dehydrogenase; (8) a a-ketoglutarate decarboxylase; (9) a 4-hydroxybutyryl CoA:acetyl-CoA transferase; (10) a butyrate kinase; (11) a phosphotransbutyrylase; (12) an aldehyde dehydrogenase; and (13) an alcohol dehydrogenase. Preferred pathways include those from alpha-ketoglutarate, e.g. steps 8, 7, 9, 12 and 13; steps 3, 6, 7, 9, 12 and 13; and steps 1, 6, 7, 9, 12, 13. In an alternative, a single protein can comprise the activities of steps 12 and 13. Specific enzymes, classes of enzymes and sources of enzymes and their genes can be found in WO2008115840A2, for example.

Enzymes, genes and methods for engineering pathways from succinate and succinyl-CoA to various products, such as BDO, into a microorganism, are now known in the art (see, e.g., U.S. Publ. No. 2011/0201089). A set of BDO pathway enzyme s represents a group of enzymes that can convert succinate or alpha-ketoglutarate to BDO as shown in FIG. 7. The additional reducing equivalents obtained from the MeDH pathway, as disclosed herein, improve the yields of all these products when utilizing carbohydrate-based feedstock. For example, BDO can be produced from succinyl-CoA via previously disclosed pathways (see for example, Burk et al., WO 2008/115840).

In other embodiments, the organism having (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), either alone or in combination with a BDO or a methacrylic acid pathway, pathway, as provided herein, may further comprise a second formaldehyde assimilation pathway (FAP). The second FAP can also utilize formaldehyde, for example, formaldehyde that not utilized by the primary pathway, in the formation of intermediates of certain central metabolic pathways that can be used, for example, in the formation of biomass.

With reference to FIG. 4, in the second formaldehyde assimilation pathway, the organism comprises at least one exogenous nucleic acid encoding a formaldehyde assimilation pathway enzyme that is different than the gapN, hexulose-6-phosphate synthase, 6-phospho-3-hexuloisomerase, and phosphoketolase proteins. Enzymes of the second formaldehyde assimilation pathway can be expressed in a sufficient amount to produce an intermediate of glycolysis and/or a metabolic pathway that can be used in the formation of biomass. In one embodiment, the second formaldehyde assimilation pathway enzyme is expressed in a sufficient amount to produce an intermediate of glycolysis. In another embodiment, the second formaldehyde assimilation pathway enzyme is expressed in a sufficient amount to produce an intermediate of a metabolic pathway that can be used in the formation of biomass. In some of the embodiments, the second formaldehyde assimilation pathway comprises a dihydroxyacetone (DHA) synthase or a DHA kinase. The preferred DHA pathway is DHA Route 1 in FIG. 4, which is a combination of DHA (dihydroxyacetone) synthase, e.g. EC 2.2.1.3 (Step 6) and F6P (fructose-6-phosphate) aldolase (Step 7). In one embodiment, the intermediate is a DHA, a DHA phosphate, or a combination thereof. In certain embodiments, the organism comprises two exogenous nucleic acids, each encoding a second formaldehyde assimilation pathway enzyme.

In addition to engineered microbial cell having at (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), the cell can also possess a pathway that proceeds through dihydroxyacetone (DHA). Both the enzyme of the RuMP pathway and the DHA pathway can be for the detoxification and assimilation of formaldehyde. As shown in FIG. 4, a transketolase first transfers a glycoaldehyde group from xylulose-5-phosphate to formaldehyde (Step 6, FIG. 4), resulting in the formation of DHA and G3P, which is an intermediate in glycolysis. The DHA obtained from DHA synthase is then further phosphorylated to form DHA phosphate (DHAP) by a DHA kinase. DHAP can be assimilated into glycolysis and several other pathways. Alternatively, DHA and G3P can be converted by fructose-6-phosphate aldolase to form fructose-6-phosphate (F6P) (FIG. 4, step 7).

In some embodiments, in addition to the cell having at least (a) exogenous enzyme A that (ai) is capable of converting G3P to 3PG, that (aii) has at least 50% sequence identity to SEQ ID NO:1, wherein enzyme A is capable of reducing NADP to NADPH, or (aiii) a fructose-1,6-bisphosphatase, or (ai) and (aiii), or (aii) and (aiii); and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), the cell can also possess a pathway that proceeds through hexose-6-phosphate (H6P) as depicted in FIG. 4. The pathway that proceeds through hexose-6-phosphate (H6P) can optionally use MeDH. For example, an engineered cell of the disclosure can include a MeDH enzyme of the DHA pathway and a MeDH enzyme of the RuMP pathway, which can be for the detoxification and assimilation of formaldehyde.

Those skilled in the art will understand that an organism can be engineered that secretes the biosynthesized compounds when grown on a carbon source such as a methanol alone or combined with other carbohydrates. Such compounds include, for example, methacrylic acid, BDO, and any of the intermediate metabolites in the methacrylic acid or BDO pathways. All that is required is to engineer in one or more of the required enzyme or protein activities to achieve biosynthesis of the desired compound or intermediate including, for example, inclusion of some or all of the methacrylic acid or BDO biosynthetic pathways. Accordingly, provided herein is an organism that produces and/or secretes methacrylic acid or BDO when grown on a carbohydrate or other carbon source and produces and/or secretes any of the intermediate metabolites shown in the product pathways when grown on a carbohydrate or other carbon source. The methacrylic acid or BDO producing microbial organisms provided herein can initiate synthesis from an intermediate. The same holds true for intermediates in the formaldehyde assimilation.

In one embodiment, the carbon source is methanol or formate. In certain embodiments, methanol is used as a carbon source. In other embodiments, formate is used as a carbon source. In specific embodiments, methanol is used as a carbon source in the organisms provided herein, either alone or in combination with the product pathways provided herein.

In one embodiment, the carbon source comprises methanol, and sugar (e.g., glucose) or a sugar-containing biomass. In another embodiment, the carbon source comprises formate, and sugar (e.g., glucose) or a sugar-containing biomass. In one embodiment, the carbon source comprises methanol, formate, and sugar (e.g., glucose) or a sugar-containing biomass. In specific embodiments, the methanol or formate, or both, in the fermentation feed is provided as a mixture with sugar (e.g., glucose) or sugar-comprising biomass. In certain embodiments, sugar is provided for sufficient strain growth. In some embodiments, the sugar (e.g., glucose) is provided at a molar concentration ratio of methanol to sugar of from 200:1 to 1:200. In certain embodiments, the carbon source comprises formate and a sugar (e.g., glucose). In some embodiments, the sugar (e.g., glucose) is provided at a molar concentration ratio of formate to sugar of from 200:1 to 1:200. In certain embodiments, the carbon source comprises a mixture of methanol and formate, and a sugar (e.g., glucose). In certain embodiments, sugar is provided for sufficient strain growth. In some embodiments, the sugar (e.g., glucose) is provided at a molar concentration ratio of methanol and formate to sugar of from 200:1 to 1:200.

Suitable purification and/or assays to test, e.g., for the production of methacrylic acid or BDO can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The methacrylic acid or BDO or other target molecules may be separated from other components in the culture using a variety of methods well known in the art. Such separation methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. All of the above methods are well known in the art.

Examples of target molecule isolation processes include distillation for 13BDO, 14BDO, butadiene, methyl vinyl carbinol, 3-buten-1-ol, n-propanol, isopropanol, propylene, and crotyl alcohol; crystallization for 6ACA (alternatively it can be converted to caprolactam and then purified via distillation as a final step), HMDA, adipic acid or derivatives thereof, succinic acid or derivatives thereof, or any of crystallization, distillation, or extraction for methacrylic acid or derivatives thereof.

Target molecules such as 13BDO, 14BDO, butadiene, methyl vinyl carbinol n-propanol, isopropanol, propylene, crotyl alcohol; 3-buten-1-ol, 6ACA, HMDA, adipic acid or derivaties thereof, succinic acid or derivatives thereof, or methacrylic acid or derivatives thereof are chemicals used in commercial and industrial applications. In some embodiments, BDO and/or 4-HB are used in various commercial and industrial applications. Non-limiting examples of such applications include production of plastics, elastic fibers, polyurethanes, polyesters, including polyhydroxyalkanoates such as P4HB or co-polymers thereof, PTMEG and polyurethane-polyurea copolymers, referred to as spandex, elastane or Lycra™, nylons, and the like.

Moreover, BDO and/or 4-HB are also used as a raw material in the production of a wide range of products including plastics, elastic fibers, polyurethanes, polyesters, including polyhydroxyalkanoates such as P4HB or co-polymers thereof, PTMEG and polyurethane-polyurea copolymers, referred to as spandex, elastane or Lycra™, nylons, and the like.

Accordingly, in some embodiments, provided are biobased plastics, elastic fibers, polyurethanes, polyesters, including polyhydroxyalkanoates such as P4HB or co-polymers thereof, PTMEG and polyurethane-polyurea copolymers, referred to as spandex, elastane or Lycra™, nylons, and the like, comprising one or more bioderived BDO and/or 4-HB or bioderived BDO and/or 4-HB intermediate thereof produced by an organism provided herein or produced using a method disclosed herein.

As used herein, the term “bioderived” means derived from or synthesized by a biological organism and can be considered a renewable resource since it can be generated by a biological organism. Such a biological organism, in particular the microbial organisms disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the disclosure. A biobased or bioderived product is in contrast to a petroleum derived product, wherein such a product is derived from or synthesized from petroleum or a petrochemical feedstock.

In some embodiments, provided herein is a culture medium comprising bioderived BDO. In some embodiments, the bioderived BDO is produced by culturing an organism having enzyme A is capable of reducing NADP to NADPH, and (b) an exogenous enzyme B which is (bi) a hexulose-6-phosphate synthase, (bii) a 6-phospho-3-hexuloisomerase, (biii) a phosphoketolase, or any combination of (bi), (bii) and (biii), and BDO pathway, as provided herein. In certain embodiments, the bioderived BDO has a carbon-12, carbon-13 and carbon-14 isotope ratio that reflects an atmospheric carbon dioxide uptake source. In one embodiment, the culture medium is separated from an organism having a fusion protein and BDO pathway.

In other embodiments, provided herein is a bioderived BDO. In some embodiments, the bioderived BDO is produced by culturing an organism having a fusion protein and BDO pathway, as provided herein. In some embodiments, the bioderived BDO has an Fm value of at least 80%, at least 85%, at least 90%, at least 95% or at least 98%. In certain embodiments, the bioderived BDO is a component of culture medium.

In certain embodiments, provided herein is a composition comprising a bioderived BDO provided herein, for example, a bioderived BDO produced by culturing an organism having a MeDH fusion protein and BDOP (BDO pathway), as provided herein. In some embodiments, the composition further comprises a compound other than said bioderived BDO. In certain embodiments, the compound other than said bioderived BDO is a trace amount of a cellular portion of an organism having a fusion protein and a BDO pathway, as provided herein.

In some embodiments, provided herein is a biobased product comprising a bioderived BDO provided herein. In certain embodiments, the biobased product is a plastic, elastic fiber, polyurethane, polyester, polyhydroxyalkanoate, poly-4-HB, co-polymer of poly-4-HB, poly(tetramethylene ether) glycol, polyurethane-polyurea copolymer, spandex, elastane, Lycra™, or nylon. In certain embodiments, the biobased product comprises at least 5% bioderived BDO. In certain embodiments, the biobased product is (i) a polymer, THF or a THF derivative, or GBL or a GBL derivative; (ii) a plastic, elastic fiber, polyurethane, polyester, polyhydroxyalkanoate, poly-4-HB, co-polymer of poly-4-HB, poly(tetramethylene ether) glycol, polyurethane-polyurea copolymer, spandex, elastane, Lycraim, or nylon; (iii) a polymer, a resin, a fiber, a bead, a granule, a pellet, a chip, a plastic, a polyester, a thermoplastic polyester, a molded article, an injection-molded article, an injection-molded part, an automotive part, an extrusion resin, an electrical part and a casing; and optionally where the biobased product is reinforced or filled and further where the biobased product is glass-reinforced or -filled or mineral-reinforced or -filled; (iv) a polymer, wherein the polymer comprises polybutylene terephthalate (PBT); (v) a polymer, wherein the polymer comprises PBT and the biobased product is a resin, a fiber, a bead, a granule, a pellet, a chip, a plastic, a polyester, a thermoplastic polyester, a molded article, an injection-molded article, an injection-molded part, an automotive part, an extrusion resin, an electrical part and a casing; and optionally where the biobased product is reinforced or filled and further where the biobased product is glass-reinforced or -filled or mineral-reinforced or -filled; (vi) a THF or a THF derivative, wherein the THF derivative is polytetramethylene ether glycol (PTMEG), a polyester ether (COPE) or a thermoplastic polyurethane; (viii) a THF derivative, wherein the THF derivative comprises a fiber; or (ix) a GBL or a GBL derivative, wherein the GBL derivative is a pyrrolidone. In certain embodiments, the biobased product comprises at least 10% bioderived BDO. In some embodiments, the biobased product comprises at least 20% bioderived BDO. In other embodiments, the biobased product comprises at least 30% bioderived BDO. In some embodiments, the biobased product comprises at least 40% bioderived BDO. In other embodiments, the biobased product comprises at least 50% bioderived BDO. In one embodiment, the biobased product comprises a portion of said bioderived BDO as a repeating unit. In another embodiment, provided herein is a molded product obtained by molding the biobased product provided herein. In other embodiments, provided herein is a process for producing a biobased product provided herein, comprising chemically reacting said bioderived-BDO with itself or another compound in a reaction that produces said biobased product. In certain embodiments, provided herein is a polymer comprising or obtained by converting the bioderived BDO. In other embodiments, provided herein is a method for producing a polymer, comprising chemically or enzymatically converting the bioderived BDO to the polymer. In yet other embodiments, provided herein is a composition comprising the bioderived BDO, or a cell lysate or culture supernatant thereof.

BDO is a valuable chemical for the production of high performance polymers, solvents, and fine chemicals. It is the basis for producing other high value chemicals such as tetrahydrofuran (THF) and gamma-butyrolactone (GBL). The value chain is comprised of three main segments including: (1) polymers, (2) THF derivatives, and (3) GBL derivatives. In the case of polymers, BDO is a comonomer for polybutylene terephthalate (PBT) production. PBT is a medium performance engineering thermoplastic used in automotive, electrical, water systems, and small appliance applications. Conversion to THF, and subsequently to polytetramethylene ether glycol (PTMEG), provides an intermediate used to manufacture spandex products such as LYCRA® fibers. PTMEG is also combined with BDO in the production of specialty polyester ethers (COPE). COPEs are high modulus elastomers with excellent mechanical properties and oil/environmental resistance, allowing them to operate at high and low temperature extremes. PTMEG and BDO also make thermoplastic polyurethanes processed on standard thermoplastic extrusion, calendaring, and molding equipment, and are characterized by their outstanding toughness and abrasion resistance. The GBL produced from BDO provides the feedstock for making pyrrolidones, as well as serving the agrochemical market. The pyrrolidones are used as high performance solvents for extraction processes of increasing use, including for example, in the electronics industry and in pharmaceutical production. Accordingly, provided herein is bioderived BDO produced according to the methods described herein and biobased products comprising or obtained using the bioderived BDO.

In some embodiments, the carbon feedstock and other cellular uptake sources such as phosphate, ammonia, sulfate, chloride and other halogens can be chosen to alter the isotopic distribution of the atoms present in BDO and/or 4-HB or any BDO and/or 4-HB pathway intermediate. The various carbon feedstock and other uptake sources enumerated above will be referred to herein, collectively, as “uptake sources.” Uptake sources can provide isotopic enrichment for any atom present in the product BDO and/or 4-HB or BDO and/or 4-HB pathway intermediate, or for side products generated in reactions diverging away from a BDO and/or 4-HB pathway. Isotopic enrichment can be achieved for any target atom including, for example, carbon, hydrogen, oxygen, nitrogen, sulfur, phosphorus, chloride or other halogens. The same holds true for the MMPs and FAPs, as well as intermediates thereof, provided herein.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target isotopic ratio of an uptake source can be obtained by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

Isotopic enrichment is readily assessed by mass spectrometry using techniques known in the art such as Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC) and/or high performance liquid chromatography (HPLC).

Accordingly, in some embodiments, provided are BDO and/or 4-HB or a BDO and/or 4-HB pathway intermediate thereof that has a carbon-12, carbon-13, and carbon-14 ratio that reflects an atmospheric carbon, also referred to as environmental carbon, uptake source.

Further, the disclosure relates, in part, to biologically produced BDO and/or 4-HB or BDO and/or 4-HB intermediate thereof as disclosed herein, and to the products derived therefrom, wherein the BDO and/or 4-HB or a BDO and/or 4-HB intermediate thereof has a carbon-12, carbon-13, and carbon-14 isotope ratio of about the same value as the CO₂ that occurs in the environment.

Those skilled in the art will understand that an organism can be engineered that secretes the biosynthesized compounds when grown on a carbon source such as a methanol alone or combined with other carbohydrates. Such compounds include, for example, BDO and any of the intermediate metabolites in the BDOP. All that is required is to engineer in one or more of the required enzyme or protein activities to achieve biosynthesis of the desired compound or intermediate including, for example, inclusion of some or all of the BDO biosynthetic pathways. Accordingly, provided herein is an organism that produces and/or secretes BDO when grown on a carbohydrate or other carbon source and produces and/or secretes any of the intermediate metabolites shown in the BDOP when grown on a carbohydrate or other carbon source. The BDO producing microbial organisms provided herein can initiate synthesis from an intermediate. The same holds true for intermediates in the formaldehyde assimilation.

### Example 1—GapN Versus GapA Kinetic Comparison

Catalytic constants for E. coli GapA and B. methanolicus GapN were determined and are shown in Table 18.

TABLE 18
GapN vs. GapA kinetic comparison

| | | | k_{cat} | Kₘ | k_{cat}/K_{M} |
|---|---|---|---|---|---|
| | organism/ | | (s⁻¹) | (mM) | (s⁻¹mM⁻¹) |
| cds | enzyme | fixed | G3P | G3P | G3P |
| 87A | EC GapA | NAD | 19 ± 1 | 2.0 ± 0.3 | 9.5 |
| 10662A | BM PB1 | NADP | 8.9 ± 0.8 | 1.1 ± 0.3 | 7.6 |
| | GapN | | | | |
| 10663A | BM MGA3 | NADP | 9.5 ± 0.3 | 1.6 ± 0.1 | 6.0 |
| | GapN | | | | |
| | | | NADP | NADP | NADP |
| 10662A | BM PB1 GapN | G3P | 9.7 ± 0.8 | 0.32 ± 0.07 | 31 |
| 10663A | BM MGA3 | G3P | 8.2 ± 0.5 | 0.27 ± 0.04 | 30 |
| | GapN | | | | |

### Example 2—Intracellular Metabolite Profiles are Achieved Due to GapN Expression

The intracellular metabolite profiles of a E. coli strain where B. methanolicus GapN enzyme (Accession WP_003351798; 95% ID to SEQ ID: 1) was expressed in conjunction with hexulose-6-phosphate synthase (Hps) (SEQ ID:2) and 6-phospho-3-hexuloisomerase (Phi)(SEQ ID:3) was compared with a strain having gapA expressing and expression of the same Hps and Phi enzymes. This strain also has a deletion of gapA, expresses a methanol dehydrogenase, and expresses fba, glpX, rpe and tkt from B. methanolicus. Expression of gapN resulted in increased metabolite levels for the relevant RuMP cycle metabolites compared to the strain with gapA expression without gapN. This indicates the benefit of gapN expression for RuMP cycle activity and methylotrophy. Table 19 shows the fold-change in increase of key RuMP cycle metabolites involved in and needed for methylotrophy.

TABLE 19
Fold change increase of RuMP cycle metabolites
in gapN strain compared to gapA strain

| | Fold change increase in gapN |
|---|---|
| Metabolite | strain compared to gapA strain |
| Fructose-6-phosphate | 1.83 |
| Fructose-1,6-bisphosphate | 14.86 |
| Glyceraldehyde-3-phosphate | 1.41 |
| Dihydroxyacetone phosphate | 1.41 |
| Sedoheptulose-1,7- | 13.93 |
| bisphosphate | |
| Sedoheptulose-7-phosphate | 2.07 |
| Ribulose-5-phosphate | 1.85 |

### Example 3—Synthetic Methylotrophy Demonstrated in Cultures

E. coli strain (overexpression of gapN (Accession WP_003351798; 95% ID to SEQ ID: 1), deletion of gapA, expression of other enzymes, methanol dehydrogenase, Hps (SEQ ID:2), Phi (SEQ ID:3), Fba, GlpX, Tkt) and its parent with gapA expressing were cultivated in a chemostat culture with glucose and methanol. The chemostat bioreactor was initiated as batch cultivation mode and operated at aerobic conditions with glucose as carbon source, the temperature controlled at 35 degrees C. and dissolved oxygen controlled above 20% while changing airflow, and agitation. pH was controlled constant at 6.95 by automatic addition of base or acid when necessary. Transition into methanol growth phase was performed operating fermenters as continuous by feeding medium containing methanol as sole carbon source targeting a dilution rate of 0.1 h-1. The methanol feed started before glucose depletion and the methanol growth kinetics were measured only after residual glucose was completely consumed. Cell growth during fermentation was monitored by measuring optical density of the culture at 600 nm using spectrophotometer.

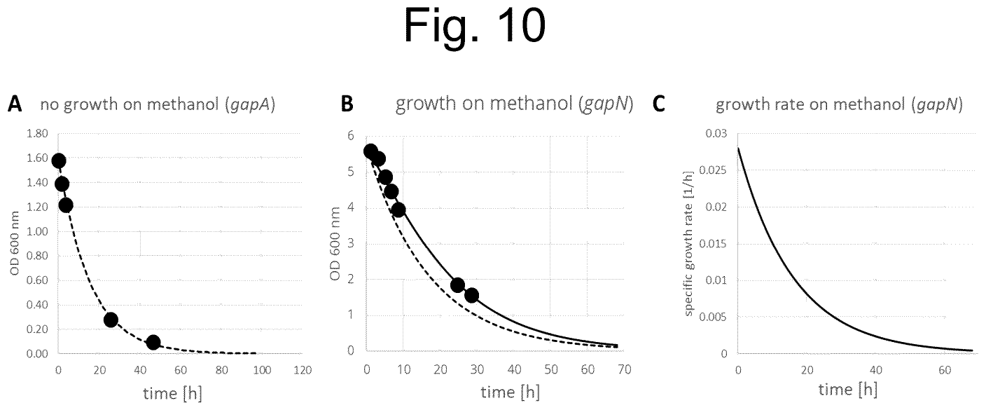

Upon transition of feed to sole carbon source of methanol, the strain with gapA but not gapN did not show growth whereas the strain with the expression of gapN exhibited growth on methanol for 30 hours. The dilution patterns for the strain with gapA fits the expectation for no growth (FIG. 10A), whereas the gapN strain exhibits synthetic methylotrophy (FIG. 10B). The observed growth rate for this strain is shown in FIG. 10C. Furthermore, the intracellular flux distribution calculated with ¹³C-methanol confirms the activity of a complete RuMP cycle through the presence of multiple fully ¹³C-labeled RuMP cycle metabolites (data not shown).

Additionally, the above GapN-expressing E. coli strains expressing an alternative Hps (41% ID to SEQ ID:2; Accession. WP_054009748.1) and Phi (33% ID to SEQ ID:3; Accession. WP_012298822.1) also demonstrated complete RuMP cycle activity evidenced by formaldehyde consumption as well as fully labeled RuMP cycle metabolites from ¹³C-methanol.

### Example 4—Overexpression of Fba, Fba2, GlpX

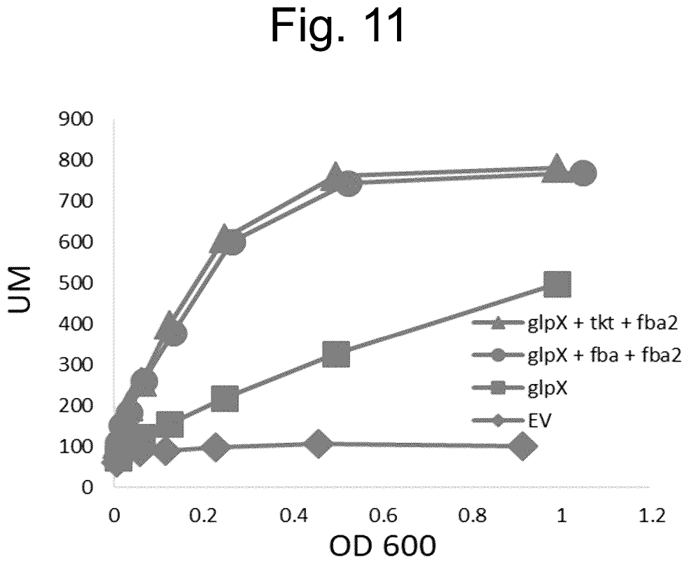

The effect of overexpression of GlpX (Accession. WP_003352248.1) on synthetic methylotrophy is demonstrated in FIG. 11 wherein strains with additional GlpX expressed on a plasmid compared to the parent strain exhibited increased formaldehyde consumption in the absence of other carbon sources. This formaldehyde consumption rate is further enhanced by the additional expression of Fba (Accession. EU77616.1), Fba2 (Accession. EU80286.1), and Tkt (Accession. EU77615.1). Formaldehyde consumption measured using NASH assay serve as a proxy for measuring RuMP cycle activity. To measure the formaldehyde consumption, the bacterial cells were grown for 5.5 hrs in Luria Broth and 2% glucose at 35 degrees C. in a 2 mL 24-deep-well plate. The cells were subsequently transferred to minimal media with 2% glucose and 4 mM MgSO4 and grown overnight in a 2 mL 48-deep-well plate. Cells were normalized to OD 0.5 and resuspended in minimal media 2% glucose, 4 mM MgSO4 and 1 mM formaldehyde in a 1.2 mL 24-deep-well plate. Samples were taken and spun down for 10 minutes at maximum rpm. Seventy-five uL of NASH B reagent was added to 75 μL of supernatant and mixed in a 96 well Costar plate and incubated at 37 degrees C. for 40 mins. Absorbance values were measured at 412 nM to obtain formaldehyde concentration.

### Example 5—Deletion of TalABC

There are two possible routes for carbon flux through the RuMP cycle, either through transaldolase (talABC) or SBPase encoded by fba and glpX as discussed herein. The transaldolase-using variant of the RuMP cycle creates a thermodynamic uphill for the generation of the critical sedoheptulose-7-phosphate with a standard delta G of 0.7 kJ/mol, whereas in contrast the SBPase-using variant uses a GlpX reaction that provides a very significant thermodynamic advantage for the generation of sedoheptulose-7-phosphate (standard delta G of −35.3 kJ/mol). To complete the RuMP cycle and regenerate ribulose-5-phosphate from glyceraldehyde-3-phosphate and dihydroxyacetone phosphate, this thermodynamic advantage of the SBPase variant provided by Fba (e.g. Accession. EU77616.1) and GlpX (e.g. Accession. WP_003352248.1) is likely critical. The importance of this is further highlighted by the calculation of delta G′ from measured intracellular metabolite levels in a natural methylotroph (B. methanolicus) as well as an engineered E. coli expressing GapN (95% ID to SEQ ID: 1; Accession WP_003351798), Hps (SEQ ID: 2), Phi (SEQ ID: 3). In the presence of transaldolase (talABC), the thermodynamics are hugely unfavorable whereas the deletion of talABC and overexpression of glpX and fba shows more favorable thermodynamics (Table 20).

TABLE 20
Gibbs free energy calculated from measured intracellular metabolite
levels in B. methanolicus as well as engineered E. coli.

| | | E. coli engineered |
|---|---|---|
| Delta G’ (kJ/mol) | B. methanolicus | with gapN |
| RuMP via transaldolase | 19 | 28 |
| RuMP via Fba & GlpX | −17 | −2 (talABC deleted) |

### Example 6—Expression of rpi, rpe, tkt, tkt2 from B. methanolicus

As shown in Table 21, transketolases (Tkt's) of B. methanolicus were determined to have significantly lower Michaelis constant Kₘ which is the substrate concentration at which the reaction rate if half of the maximum rate. As disclosed herein, B. methanolicus transketolases kinetically favor the RuMP cycle flux for ribulose 5-phosphate regeneration, which is appears essential for methanol and formaldehyde assimilation. Alternatively, other transketolases can be engineered or discovered that have the desired Km.

For other reversible enzymes of the RuMP cycle such as ribose 5-phosphate isomerase (RpiAB) and ribulose-phosphate 3-epimerase (Rpe), a similar consideration was discovered and applied, i.e., the corresponding B methanolicus enzymes have better kinetic properties than E. coli homologs. E. coli has two isozymes of ribose 5-phosphate isomerase A (RpiA) and B (RpiB), where RpiA is the major enzyme in E. coli. The Michaelis constant for its substrate ribose 5-phosphate is 4.4 mM for RpiA and 0.83 mM for RpiB [1]. Therefore, deletion of rpiA and overexpression of rpiB from either B. methanolicus or E. coli should kinetically favor the product formation which is ribulose 5-phosphate. References cited in this section and Table 21 are: [1] Essenberg and Cooper, “Two Ribose-5-Phosphate Isomerases from Escherichia coli K12: Partial Characterisation of the Enzymes and Consideration of Their Possible Physiological Roles,” European Journal of Biochemistry, vol. 55, no. 2, pp. 323-32, 1975; [2] Markert et al., “Characterization of two transketolases encoded on the chromosome and the plasmid pBM19 of the facultative ribulose monophosphate cycle methylotroph Bacillus methanolicus,” BMC Microbiology, vol. 14, no. 7, 2014; and [3] Sprenger et al., “Transketolase A of Escherichia coli K12,” European Journal of Biochemistry, vol. 230, no. 2, pp. 525-32, 1995.

TABLE 21
RpiB transketolase Michaelis constant K_{M} comparison
between B. methanolicus and E. coli for
the reaction substrates and products.

K_{M} [mM]

| | B. methanolicus [2] | E. coli [3] |
|---|---|---|
| Substrates | | |
| fructose 6-phosphate | 0.72 | 1.1 |
| glyceraldehyde 3-phosphate | 0.92 | 2.1 |
| sedoheptulose 7-phosphate | | 4 |
| Products | | |
| erythrose 5-phosphate | | 0.09 |
| ribose 5-phosphate | 0.12 | 1.4 |
| xylulose 5-phosphate | 0.15 | 0.16 |

1. An engineered microorganism having synthetic or enhanced methylotrophy comprising:
(a) exogenous enzyme A that is a NADP-dependent glyceraldehyde-3-phosphate dehydrogenase having at least 85% sequence identity to SEQ ID:1 or SEQ ID NO:11, wherein said exogenous enzyme A is capable of converting glyceraldehyde 3-phosphate (G3P) to 3-phosphoglycerate (3PG), and is capable of reducing NADP to NADPH and
(b) an exogenous enzyme B which is (bi) a phosphoketolase, (bii) a hexulose-6-phosphate synthase, (biii) 6-phospho-3-hexuloisomerase, or any combination of (bi), (bii) and (biii).

2. The engineered microorganism of claim 1 comprising the (a) exogenous enzyme A, and the (bi) exogenous phosphoketolase, and optionally the (bii) exogenous hexulose-6-phosphate synthase, and the (biii) exogenous 6-phospho-3-hexuloisomerase.

3-9. (canceled)

10. The engineered microorganism of claim 1 wherein the phosphoketolase is:
(1) an exogenous fructose-6-phosphate phosphoketolase and the microorganism further comprises (a) a phosphotransacetylase, or (b) an acetyl-CoA transferase, an acetyl-CoA synthetase or an acetyl-CoA ligase,
(2) an exogenous xylulose-5-phosphate phosphoketolase and the engineered microorganism further comprises (a) a phosphotransacetylase, or (b1) an acetate kinase deletion and (b2) an acetyl-CoA transferase, and acetyl-CoA synthetase, or an acetyl-CoA ligase, or
both (1) and (2).

11-14. (canceled)

15. The engineered microorganism of claim 1, wherein the exogenous enzyme B comprises hexulose-6-phosphate synthase having at least 85% identity to SEQ ID NO: 2 (Bacillus methanolicus MGA HPS).

16. (canceled)

17. (canceled)

18. The engineered microorganism of claim 1 further comprising a NAD⁺-dependent methanol dehydrogenase (MDH).

19-21. (canceled)

22. The engineered microorganism of claim 1 comprising a pathway from xyulose-5-phosphate (Xu5P) to fructose-6-phosphate (F6P) or from ribulose-5-phosphate (Ru5P) to F6P comprising dihydroxyacetone synthase and fructose-6-phosphate aldolase.

23.-25. (canceled)

26. The engineered microorganism of claim 1 comprising one or more modification(s) that (a) attenuates or eliminates an endogenous enzyme activity in a pathway leading from glyceraldehyde-3-phosphate to phosphoenolpyruvate (PEP), wherein the one or more modifications:
attenuates or eliminates an endogenous NAD-dependent glyceraldehyde-3-phosphate dehydrogenase activity that converts glyceraldehyde-3-phosphate to 1,3-bisphosphoglycerate wherein the modification is optionally a deletion of or mutation in gapA;
attenuates or eliminates an endogenous phosphoglycerate kinase activity (pgk) that converts 1,3-bisphosphoglycerate to 3-phosphoglycerate, wherein the modification is optionally a deletion of or mutation in pgk;
attenuates or eliminates an endogenous phosphoglycerate mutase activity that converts 3-phosphoglycerate to 2-phosphoglycerate, wherein the modification is optionally a deletion of or mutation in gpmA or gpmM; or
attenuates or eliminates an endogenous enolase activity that converts 2-phosphoglycerate to phosphoenolpyruvate, wherein the modification is optionally a deletion of or mutation in eno.

27-31. (canceled)

32. The engineered microorganism of claim 1 further comprising:
a modification that attenuates or eliminates endogenous transaldolase activity which converts glyceraldehyde-3-phosphate and sedoheptulose-7-phosphate to erythrose-4-phosphate and fructose-6-phosphate, optionally wherein the modification that attenuates or eliminates transaldolase activity is a deletion or mutation to a nucleic acid encoding at least one of talA, talB, and talc; or
a modification that attenuates or eliminates endogenous ATP-dependent 6-phosphofructokinase that converts D-fructose 6-phosphate to fructose 1,6-bisphosphate, wherein optionally the modification that attenuates or eliminates transaldolase activity is a deletion or mutation to a nucleic acid encoding pfkA.

33-35. (canceled)

36. The engineered microorganism of claim 1 further comprising:
an exogenous ATP-dependent 6-phosphofructokinase that converts D-fructose 6-phosphate to fructose 2,6-bisphosphate, optionally wherein the ATP-dependent 6-phosphofructokinase has a sequence identity of 85% or greater, 90% or greater, or 95% or greater to Bacillus methanolicus PB1 pfk2 (SEQ ID NO.:5);
an exogenous ribulose-phosphate 3-epimerase that converts D-fructose 6-phosphate to fructose 2,6-bisphosphate, wherein the ribulose-phosphate 3-epimerase optionally has a sequence identity of 85% or greater, 90% or greater, or 95% or greater to Bacillus methanolicus MGA3 re (SEQ ID NO.:6);
an exogenous ribose-5-phosphate isomerase that converts ribose-5-phosphate to ribulose 5-phosphate, wherein the ribose-5-phosphate isomerase optionally has a sequence identity of 85% or greater, 90% or greater, or 95% or greater to Bacillus methanolicus MGA3 rpiB (SEQ ID NO.:7)
an exogenous endogenous transketolase that converts sedoheptulose 7-phosphate and D-glyceraldehyde 3-phosphate to D-ribose 5-phosphate and D-xylulose 5-phosphate, wherein the transketolase optionally has a sequence identity of 85% or greater, 90% or greater, or 95% or greater to Bacillus methanolicus PB1 tkt (SEQ ID NO.:8);
an exogenous fructose-bisphosphate aldolase that converts dihydroxyacetone phosphate (DHAP) with glyceraldehyde 3-phosphate (G3P) to form fructose 1,6-bisphosphate (FBP), wherein the fructose-bisphosphate aldolase optionally has a sequence identity of 85% or greater, 90% or greater, or 95% or greater to Bacillus methanolicus PB1 fba2 (SEQ ID NO.:9);
an exogenous glucose-6-phosphate 1-dehydrogenase that catalyzes the oxidation of glucose 6-phosphate to 6-phosphogluconolactone, wherein the exogenous glucose-6-phosphate 1-dehydrogenase optionally has a sequence identity of 85% or greater, 90% or greater, or 95% or greater to Bacillus methanolicus MGA3 zwf2 (SEQ ID NO.:10);
a modification that increases acetyl coA synthetase activity, wherein the modification that increases acetyl coA synthetase activity is optionally upregulation of expression or activity of acs; or
a modification that increases phosphate acetyl transferase activity, wherein the modification that increases phosphate acetyl transferase activity is optionally upregulation of expression or activity of pta.

37. (canceled)

38. The engineered microorganism of claim 1 further comprising a modification that attenuates or eliminates:
an endogenous ribulose-phosphate 3-epimerase that converts D-ribulose 5-phosphate to D-xylulose 5-phosphate wherein optionally the modification that attenuates or eliminates ribulose-phosphate 3-epimerase activity is a deletion or mutation to a nucleic acid encoding rpe;
an endogenous ribose-5-phosphate isomerase that converts ribose-5-phosphate to ribulose 5-phosphate, wherein optionally the modification that attenuates or eliminates ribose-5-phosphate isomerase activity is preferably a deletion or mutation to a nucleic acid encoding rpiA;
an endogenous transketolase that converts sedoheptulose 7-phosphate and D-glyceraldehyde 3-phosphate to D-ribose 5-phosphate and D-xylulose 5-phosphate, wherein optionally the modification that attenuates or eliminates transketolase activity is a deletion or mutation to a nucleic acid encoding tktA, tktB, or both;
a fructose-bisphosphate aldolase that converts dihydroxyacetone phosphate (DHAP) with glyceraldehyde 3-phosphate (G3P) to form fructose 1,6-bisphosphate (FBP), wherein optionally the modification that attenuates or eliminates fructose-bisphosphate aldolase activity is a deletion or mutation to a nucleic acid encoding fbaA, fbaB, or both
a glucose-6-phosphate 1-dehydrogenase activity that catalyzes the oxidation of glucose 6-phosphate to 6-phosphogluconolactone, or to 2-keto-3-deoxy-6-phosphogluconate (KDPG) aldolase that converts 2-keto-3-deoxy-6-phosphogluconate (KDPG) to pyruvate and D-glyceraldehyde-3-phosphate, wherein optionally the modification that attenuates or eliminates glucose-6-phosphate 1-dehydrogenase activity is a deletion or mutation to a zwf gene;
a methyl glyoxal synthase activity, wherein optionally the modification that attenuates or eliminates methyl glyoxal synthase activity is a deletion or mutation to a nucleic acid encoding mgsA;
deoxyribose phosphate aldolase activity, wherein optionally the modification that attenuates or eliminates deoxyribose phosphate aldolase activity is a deletion or mutation to a deoC gene; or
acetate kinase activity, wherein the modification that attenuates or eliminates acetate kinase activity is optionally a deletion or mutation to a nucleic acid encoding ackA.

39-67. (canceled)

68. The engineered microorganism of claim 1 further comprising a pathway capable of producing a bioderived compound or a pathway to a bioderived compound that uses acetyl-CoA or an amino acid precursor as a metabolite, wherein said bioderived compound is selected from the group consisting of:
(i) 1,4-butanediol or an intermediate thereto, wherein said intermediate is optionally 4-hydroxybutanoic acid (4-HB) or gamma-butyrolactone;
(ii) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol), isoprene, or 3-buten-1-ol;
(iii) 1,3-butanediol or an intermediate thereto, wherein said intermediate is optionally 3-hydroxybutyrate (3-HB), 3-hydroxy pent-4-enoate, 2,4-pentadienoate, crotyl alcohol or 3-buten-1-ol;
(iv) adipate, 6-aminocaproic acid, caprolactam, hexamethylenediamine, levulinic acid or an intermediate thereto, wherein said intermediate is optionally adipyl-CoA or 4-aminobutyryl-CoA;
(v) methacrylic acid or an ester thereof, 3-hydroxyisobutyrate, 2-hydroxyisobutyrate, or an intermediate thereto, wherein said ester is optionally methyl methacrylate or poly(methyl methacrylate);
(vi) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto;
(vii) succinic acid or an intermediate thereto;
(viii) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol); and
(ix) an isoprenoid, optionally the isoprenoid is isoprene, or an intermediate thereto.

69-77. (canceled)

78. The engineered microorganism of claim 1 which is bacteria, fungi, or yeast.

79. The engineered microorganism of claim 78 that is Escherichia, Corynebacterium, Bacillus, Ralstonia, Staphylococcus, Pichia or Saccharomyces, wherein the engineered microorganism is optionally Escherichia coli.

80. (canceled)

81. A method for producing a bioderived compound, comprising culturing engineered microorganism of 68 under conditions and for a sufficient period of time to produce said bioderived compound.

82. A method for growing a non-natural microbial organism comprising culturing engineered microorganism of claim 1 in a medium comprising carbon-containing feedstock that comprises methanol.

83. (canceled)

84. (canceled)

85. The engineered microorganism of claim 1 wherein exogenous enzyme A has at least 90% sequence identity, or at least 95% sequence identity to SEQ ID NO:1 or SEQ ID NO:11.

86. An engineered microorganism having synthetic or enhanced methylotrophy comprising:
exogenous enzyme A that is a NADP-dependent glyceraldehyde-3-phosphate dehydrogenase having at least 85% sequence identity to one of the following amino acid sequences: SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26, wherein said exogenous enzyme A is capable of converting glyceraldehyde-3-phosphate (G3P) to 3-phosphoglycerate (3PG) and capable of reducing NADP to NADPH; and
an exogenous enzyme B which is (bi) a phosphoketolase, (bii) a hexulose-6-phosphate synthase, (biii) 6-phospho-3-hexuloisomerase, or any combination of (bi), (bii) and (biii).

87. The engineered microorganism of claim 86 wherein exogenous enzyme A has at least 90% sequence identity, or at least 95% sequence identity to one of SEQ ID NOs:12-26.

88. The engineered microorganism of claim 1 wherein exogenous enzyme B which is (bi) the phosphoketolase comprises one of the following amino acid sequences: SEQ ID NO:27, SEQ ID NO:28, SEQ ID NO:29, SEQ ID NO:30, SEQ ID NO:31, SEQ ID NO:32, SEQ ID NO:33, or SEQ ID NO:34.


SEQUENCE LISTING (34 sequences)

SEQ ID NO: 1 - protein, 481 aa, Bacillus methanolicus
MQVLEKDFKFLLNGEWSFSSSNQFINIYSPSTGEVVGRVPAMTKDEIDKAVAGARQAQKKWEKLAVSERAKILHAWADELVKMIDVIAPMIMNEVGKNISTARKEVTRTAEIIRYTAEEGVRIHGEFINGGSLDADSSIKTAIVEKKPLGVILAISPFNYPINLAATKIAPALIAGNAVVFKPATQGAISGLLMIQALENAGLPKGLVNTVTGKGSEIGDYIITHPFIDMISFTGGTGTGQNIARKASMIPLVLELGGKDPAIVLEDADLELAATEIVSGAFSYSGQRCTAIKRVLVLKDVAAPLIEKIKEKVEKLTVGRPEDDADITPLIDESSADFVQGLIDDALAKGASLITGNKREKNLIYPTVLSNVTKDMKVAWEEPFGPVLPCIIVDSVEEAIEIANESEFGLQASVFTSNIEKAFTIASSLDVGSVQINGRTERGPDHFPFSAVKNSGLGSQGIRQSIISMMRDKVTVINLKK

SEQ ID NO: 2 - protein, 211 aa, Bacillus methanolicus
MELQLALDLVNIEEAKQVVAEVQEYVDIVEIGTPVIKIWGLQAVKAVKDAFPHLQVLADMKTMDAAAYEVAKAAEHGADIVTILAAAEDVSIKGAVEEAKKLGKKILVDMIAVKNLEERAKQVDEMGVDYICVHAGYDLQAVGKNPLDDLKRIKAVVKNAKTAIAGGIKLETLPEVIKAEPDLVIVGGGIANQTDKKAAAEKINKLVKQGL

SEQ ID NO: 3 - protein, 184 aa, Bacillus methanolicus
MLTTEFLAEIVKELNSSVNQIADEEAEALVNGILQSKKVFVAGAGRSGFMAKSFAMRMMHMGIDAYVVGETVTPNYEKEDILIIGSGSGETKSLVSMAQKAKSIGGTIAAVTINPESTIGQLADIVIKMPGSPKDKSEARETIQPMGSLFEQTLLLFYDAVILRFMEKKGLDTKTMYGRHANLE

SEQ ID NO: 4 - protein, 382 aa, Bacillus methanolicus
MTTNFFIPPASVIGRGAVKEVGTRLKQIGAKKALIVTDAFLHSTGLSEEVAKNIREAGVDVAIFPKAQPDPADTQVHEGVDVFKQENCDSLVSIGGGSSHDTAKAIGLVAANGGRINDYQGVNSVEKPVVPVVAITTTAGTGSETTSLAVITDSARKVKMPVIDEKITPTVAIVDPELMVKKPAGLTIATGMDALSHAIEAYVAKGATPVTDAFAIQAMKLINEYLPKAVANGEDIEAREKMAYAQYMAGVAFNNGGLGLVHSISHQVGGVYKLQHGICNSVNMPHVCAFNLIAKTERFAHIAELLGENVAGLSTAAAAERAIVALERINKSFGIPSGYAEMGVKEEDIELLAKNAYEDVCTQSNPRVPTVQDIAQIIKNAM

SEQ ID NO: 5 - protein, 319 aa, Bacillus methanolicus
MKKIGVLTSGGDSPGMNAAIRAVVRKAIYHNLEVYGVYGGYLGLINGNIKKLELGSVGDIIHRGGTMLYSARCEEFKTKEGQLKGVEQLKKHGIDGLVVIGGDGSYRGAKALTELGFPCVGVPGTIDNDIPGTEHTIGFDTALNTVIDAIDKIRDTASSHERTFVVEVMGRNAGDIALWAGLAGGAEAIVIPEENYDINEIADRLKRSHERGKRHSIIIVAEGVCRGEELSKQITEATGFATWVTVLGHVQRGGSPSAFDRVLASRLGARAVELLIEGKGGRAVGIRKNQVVDYDFDEVFSNEHTLDLELCKLSKELSI

SEQ ID NO: 6 - protein, 214 aa, Bacillus methanolicus
MIKIAPSILSANFARLEEEIKDVERGGADYIHVDVMDGHFVPNITIGPLIVEAIRPVTNLPLDVHLMIENPDQYIGTFAKAGADILSVHVEACTHLHRTIQYIKSEGIKAGVVLNPHTPVSMIEHVIEDVDLVLLMTVNPGFGGQSFIHSVLPKIKQVANIVKEKNLQVEIEVDGGVNPETAKLCVEAGANVLVAGSAIYNQEDRSQAIAKIRN

SEQ ID NO: 7 - protein, 148 aa, Bacillus methanolicus
MKVAIASDHGGVNIRQEIINLLEEMGIEYEDFGCECTTSVDYPDYALPVAEKVANGEFDRGILICGTGIGMSIAANKVKGIRCALVHDVFSAKATREHNDSNILAMGERVIGPGLAREIAKTWLTTEFQGGRHSNRIEKICKYESVNL

SEQ ID NO: 8 - protein, 668 aa, Bacillus methanolicus
MFDKIDQLSITSIRTLSIDAIEKANSGHPGMPMGAAPMAYTLWTRYMNHNPKNPNWFNRDRFVLSAGHGSMLLYSLLHLSGYDVTMDDIKEFRQWGSKTPGHPEYGHTPGVEATTGPLGQGIAMAVGIGMAERHLAAVYNKDNYQIINHYTYSICGDGDLMEGVSAEAASLAGHLRLGRLIVLYDSNDISLDGELNRSFSESVEMRFKSYGWQYIRVEDGNDLEAIAKAIEEAKQDETRPTLIEVKTIIGYGSPNRAGTSDIHGSPLGAEERKLTKEAYKWTFEEDFYVPQEVYDHFKQNVIERGEAKEQEWNELFAQYKKEYPELGKQLEQAINGELPEGWDKDIPVYEVGKSIASRASSGEVLNAIAKNLPSFIGGSADLASSNKTTIKGAGDYSPESFEGKNIWFGVREFAMGAALNGMALHGGLKVFGGTFFVFSDYLRPAIRLAALMKLPVIYVFTHDSIAVGEDGPTHEPVEQLASLRAMPNLSVIRPADGNETAAAWRLAVESKDKPTALVLSRQNLPTLKGTAETAYDGVSKGAYVVSPADKDTPDALLLASGSEVGLAVEAQVRLAEEGIHVSVISMPSFDRFEAQSKEYKESVIPKNVKKRLVIEMASSLGLHRYAGDEGDVLAIDQFGASAPGEIIMEEYGFNVDNVVAKVKALLEK

SEQ ID NO: 9 - protein, 284 aa, Bacillus methanolicus
MPLVSMKDMLNHGKENGYAVGQFNINNLEFGQAILQAAEEEKSPVIIGVSVGAANYMGGFKLIVDMVKSSMDSYNVTVPVAIHLDHGPSLEKCVQAIHAGFTSVMIDGSHLPLEENIELTKRVVEIAHSVGVSVEAELGRIGGQEDDVVAESFYAIPSECEQLVRETGVDCFAPALGSVHGPYKGEPKLGFDRMEEIMKLTGVPLVLHGGTGIPTKDIQKAISLGTAKINVNTESQIAATKAVREVLNNDAKLFDPRKFLAPAREAIKETIKGKMREFGSSGKA

SEQ ID NO: 10 - protein, 484 aa, Bacillus methanolicus
MDSMTFVLFGASGDLAKRKIYPALYNLFLDQKMPQSFSVIGLGRRDWSDNEYQMRVEQSLKTFSRRFVIDSIKMKEFLSAFRFFRLDVNNEEGFRDLFEFVKKREEELNIPENRMFYLSVAPEFIGVIASNIKKSGLGTTKGWKRLVIEKPFGNDLKSAQELNESLRNVFEEDEIYRIDHYLGKPMVQNLEALEFANPVLQAIWNNQYIANVQITASETVGVEERAGYYDQAGAIRDMFQNHMLQLLMMTAMKMPERISSKDIRNEKIKVMEYLRPLLKEDVAKHVVRGQYGPGEINGEPVVGYREEPGVDASSTTDTFVAARLWIDVPFWSGVPFYIRTGKRMREKCTKIVIEFKNPLKDLYITDNMKTVPNLLVIEINPNERVSFQLNSKNPIDGKIEPVHVNFSASQKDAPEAYEILLYDALRGDSTYFAHWKEVELSWKWVQPILEAFEENILPLHHYTSGSMGPEASHKLLEEDGFNWW

SEQ ID NO: 11 - protein, 481 aa, Bacillus methanolicus
MQVLEKDHKFLLNGKWCYSSSNEFINIYSPSTGEIVGRVPAMTKDEIDKAVAGACQAQKNWEKLAVSDRAKILHAWADELVKMIDVIAPMIMNEVGKNISTAKREVIRTADIIRYTAEEGVRIHGEFINGGSLDADSSKKTAIVEKKPLGVILAISPFNYPINLAATKIAPALIAGNAVVFKPATQGAISGLLMIQALENAGLPEGLVNTVTGKGSEIGDYIITHPFIDMISFTGGTGTGQNIARKASMIPLVLELGGKDPAIVLEDADLELAAAEIVSGAFSYSGQRCTAIKRVIVLKDAAAPLIEKIKEKVEKLTVGKPEDDADITPLIDESSADFVQGLIDDALAKGATLITGNKREKNLIYPTVLSNVTKDMKVAWEEPFGPVLPCIIVDSAEEAIEIANESEFGLQASVFTTNIEKAFSIASSLDVGSVQINGRTERGPDHFPFSAVKNSGLGSQGIRQSIISMMRDKVTVINLKR

SEQ ID NO: 12 - protein, 482 aa, Alicyclobacillus pomorum
MQQVLDGVIDYHFLLNGNWGQSASNRFIDVVSPNDGCVAGRIPAMTKEEVDLAVQGAVQAQKQWGLLPVHERGQLLLNWADELAMMSDEIAEMIMKEVGKTYSAAKNEVLRTSDLIRYTVEEGKRIHGEVMTGDSFQGSNANKVAIVRKEPLGVILAISPFNYPVNLSAAKIAPALISGNTVILKPATQGSISALLMAKALDKVGLPKGVLNVVTGKGSEIGDYLVTHPSIRMISFTGGTKTGRDIAKKATMIPLVLELGGKDPAIVLEDANLDKAARHIVSGAFSYSGQRCTAIKRVLVMDSVADSLIEKVKQQVEKLTVGMPEQDAAITPLIDASSADFVNDLILDALNKGAIPITEYRRVGNLIYPLVLDHVTQDMKVAWEEPFGPVLPIIRVKNEQEAVEIANASEYGLQASIFTQDIDKAFHLASKLEVGSVQINGRTERGPDHFPFLGVKNSGMGVQGVRKSIESMTRDKVLVLNL

SEQ ID NO: 13 - protein, 482 aa, Virgibacillus necropolis
MKAEMIANTYQFLLNGEWRESLSGKTIENQSPSDNTPVGSVQAMTENEVNLAVTGAKAAQKDWANLSFSERAELLYAWADQLLEMKEEIAETIMKEVGKGYSSAEKEVVRTADFIKYTAEEGKRIHGELINGGSFNAGSANKLAMVQREPLGVILAISPFNYPVNLSAAKIAPALIAGNAVVFKPATQGAISGTLMVRALDKAGLPSGLVNLVTGKGSEIGDYLTTHPSIDLINFTGGSETGEYISKKASMIPVILELGGKDPAIVLNDADLEKAAGDIVGGGFSYSGQRCTAIKRVLVLDEVADALVDKLKDKIKALEVGMPEDNATVTPLINEKAADFVQQLVDDAVGKGATPLTDIRREGNLVYPVLLDNVTVDMTVAWEEPFGPVLPIIRVQNIEEAVIIANESEYGLQASVFTKNVEQAIRIGSELEVGSVQINGKTERGPDHFPFLGVKNSGLGAQGIRKSIESVTREKVTVLNMS

SEQ ID NO: 14 - protein, 479 aa, Bacillus cytotoxicus
MTTSNTYKFYLNGEWRESSSGQTIDIVSPYLHEVIGKVQAITRDEVDEAIASAKEAQKDWAAASLQDRAKFLYKWADELVNMQDEIADIVMKEVGKGHKDAKKEVVRTADLIRYTVDEALHMHGDSMMGDSFPGGSKSKLAIIQRAPLGVVLAISPFNYPVNLAAAKVAPALIMGNAVIFKPATQGAISGIKMVEALHKAGLPKGLVNVVTGRGSVIGDYLVEHPGINMVSFTGGTNTGAHLAKKAAMIPLVLELGGKDPGIVCEDADLTEAAKHIVSGAFSYSGQRCTAIKRVLVHENVADELVSLLKEQVAALSVGSPEQDSTIVPLIDDKSADFVQGLVDDAVENGATIIIGNKRERNLIYPTIIDNVTEEMKVAWEEPFGPILPIIRVSSDEEAIEIANKSEFGLQASVFTKDINKAFAIANKIDTGSVQINGRTERGPDHFPFIGVKGSGMGAQGIRKSLESMTREKVTVLNLV

SEQ ID NO: 15 - protein, 479 aa, Bacillus cytotoxicus
MTTSNTYKFYLNGEWRESSSGQTIDILSPYLHEVIGKVQAITRDEVDEAIAAAKEAQKDWAEASLQDRAKFLYKWADELVNMQDEIADIIMKEVGKGYKDAKKEVVRTADFIRYTVDEALHMHGESMMGDSFPGGTKSKLAIIQRAPLGVVLAIAPFNYPVNLSAAKLAPALIMGNAVIFKPATQGAISGIKMVEAIHKAGLPKGLVNVVTGRGSVIGDYLVEHEDINMVSFTGGTNTGKHLAKKAAMIPLVLELGGKDPGIVREDADLQDAANHIVSGAFSYSGQRCTAIKRVLVHENVADELVSLLKEKVAALSVGSPEQDSTIVPLIDDKSADFVQGLVDDAVEKGATIIIGNKRERNLIYPTIIDNVTEEMKVAWEEPFGPILPIIRVSSDEEAIEIANKSEFGLQASVFTKDINKAFAIANKIDTGSVQINGRTERGPDHFPFIGVKGSGMGAQGIRKSLESMTREKVTVLNLV

SEQ ID NO: 16 - protein, 484 aa, Lentibacillus amyloliquefaciens
MSEVETIKAYPFLLNGEWQNTTSERTIEIESPSGAETPGAVQAMTESEVNAAISGANEAQKNWARKSFDERAQVLHSWADQLLLMKDEIAEAIMKEAGKGLSSAEKEVVRTADFIKYTAEEGKRLHGELINGGSFNAGSANKFALVNRNPIGVVLAISPFNYPVNLSAAKIAPALIGGNAVVFKPATQGAISGTLMIEALDKAGLPSGLVNLVTGKGAEIGDHLITHPLIDLINFTGGSQTGKSISQKASMVPLILELGGKDPAIVLEDADLDKAAADIASGGFSYSGQRCTAIKRVLVQDEKADELVAKIKEKMNGLKVGAPEDSADVTPLINSKAADYVTGLIDDAVEKGAEVASGNQREGNLIYPTLLDSVTKDMQIAWEEPFGPVIPVIRVESVDEAINIANESEYGLQASIFTKNMEQAIQIGNELEVGSVQINGKTERGPDHFPFLGVKSSGVGGQGIRKSIESMTREKVTVLNMTQG

SEQ ID NO: 17 - protein, 481 aa, Bacillus cereus
MTTITSNTYKFYVNGEWRESSSGQTIDIPSPYLHEVIGQVQAITREEVDEAIASAKEAQKEWAEASLQDRAKYLYKWADELVNMQDEIADIVMKEVGKGYKDAKKEVVRTADLIRYTVDEALHMHGESMMGDSFPGGSKSKLAIVQRAPRGVILAIAPFNYPVNLSAAKLAPALIMGNAVIFKPATQGAISGIKMIEALHKAGLPKGLVNVATGRGSVIGDYLVEHPGVNMVSFTGGTNTGAHLAKKAAMIPLVLELGGKDPGIVREDADLQEAANHIVSGAFSYSGQRCTAIKRVLVHENVADELISLLKEQVAALTVGSPEQDSTIVPLIDDKSADFVQGLVDDAVEKGATIVIGNKRERNLIYPTLIDNVTEDMKVAWEEPFGPILPIIRVSSDEQAIEIANKSDFGLQASVFTKDINKAFAIANKIDTGSVQINGRTERGPDHFPFIGVKGSGMGAQGIRKSLESMTREKVTVLNLV

SEQ ID NO: 18 - protein, 506 aa, Bacillus cereus
  source 1..506: note = isolate Rock 3-44
MKNILIPPTITIYSLLQVAILGGQIMTTITSNTYKFYVNGEWRESSSGQTIDIPSPYLHEVIGQVQAITREEVDEAIASAKEAQKEWAEASLQDRAKYLYKWADELVNMQDEIADIVMKEVGKGYKDAKKEVVRTADLIRYTVDEALHMHGESMMGDSFPGGSKSKLAIVQRAPRGVILAIAPFNYPVNLSAAKLAPALIMGNAVIFKPATQGAISGIKMIEALHKAGLPKGLVNVATGRGSVIGDYLVEHPGVNMVSFTGGTNTGAHLAKKAAMIPLVLELGGKDPGIVREDADLQEAANHIVSGAFSYSGQRCTAIKRVLVHENVTDELISLLKEQVAALTVGSPEQDSTIVPLIDDKSADFVQGLVDDAVEKGATIVIGNKRERNLIYPTLIDNVTEDMKVAWEEPFGPILPIIRVSSDEQAIEIANKSDFGLQASVFTKDINKAFAIANKIDTGSVQINGRTERGPDHFPFIGVKGSGMGAQGIRKSLESMTREKVTVLNLV

SEQ ID NO: 19 - protein, 479 aa, Bacillus cereus
  source 1..479: note = isolate group
MTTSNTYKFYLNGEWRESSSGQTIDIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGIHMVSFTGGTNTGKHLAKKASMIPLVLELGGKDPGIVREDADLQDAANHIVSGAFSYSGQRCTAIKRVLVHENVADELVELVKAQVAKLSVGSPEQDSTIVPLIDDKSADFVQGLVDDAVEKGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 20 - protein, 484 aa, Virgibacillus siamensis
MNKVESIKAYPFLLNGEWKSSTSERTIEMQSPSGAGIPGKVQAMTESEVNTALTGAKDAQKEWGRKSFDERARLLYSWADQLLLMKDEIAETIMKEVGKGLASAEKEVIRTADFIKYTAEEGKRLHGELINGGSFNSGSANKFALVNRNPVGVVLAISPFNYPVNLAAAKIAPALIAGNAVVFKPATQGAISGTRMIEALDKAGLPSGLVNLVTGKGSEIGDYLITHPLVDLINFTGGSDTGKSISEKASMVPVILELGGKDPAIVLEDADLDKAASDIVSGGFSYSGQRCTAIKRVLVIDNKADKLVENIKEKMESLKVGAPEDNADVTPLINRKATDFVTGLIEDAIEKGARVVSGNHREGNLIYPTLLDCVTKNMAIAWEEPFGPVIPVIRVESVDEAIDIANESQYGLQASIFTKNMEQAIRIGNELEVGSVQINGKTERGPDHFPFLGVKSSGVGVQGIRKSIESMTREKVTVLNMSQG

SEQ ID NO: 21 - protein, 504 aa, Bacillus thuringiensis
  source 1..504: note = strain Al Hakam
MKNVIKPHTITLYSLLQVAILGGLIMTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKASMIPLVLELGGKDPGIVREDADLQDAANHIVSGAFSYSGQRCTAIKRVLVHENVADELVSLVKEQVAKLSVGSPEQDSTIVPLIDDKSADFVQGLVDDAVEKGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 22 - protein, 504 aa, Bacillus mycoides
  source 1..504: note = isolate Rock 1-4
MKNVLKPPTITIYSLLQVAILGGQIMTTSNTYKFYLNGEWRESSSGQTIDIPSPYLHEVIGKVQAITREEVDEAIKSAQQAQKEWAEASLQDRAKYLYKWADELVNMQDEIADIVMKEVGKGYKDAKKEVVRTADLIRYTVDEALHMHGESMMGDSFPGGSKSKLAIIQRAPRGVVLAIAPFNYPVNLSAAKLAPALIMGNAVIFKPATQGAISGIKMVEALHKAGLPKGLVNVATGRGSVIGDYLVEHPGVNMVSFTGGTNTGAHLAKKAAMIPLVLELGGKDPGIVREDADLGEAAKHIVSGAFSYSGQRCTAIKRVLVHENVADELVSLLKDQVAELTVGSPEQDSTIVPLIDDKSADFVQGLVDDAVEKGATIVIGNKRERNLIYPTLIDNVTEDMKVAWEEPFGPILPIIRVSSDEQAIEIANKSDFGLQASVFTKDINKAFAIANKIDTGSVQINGRTERGPDHFPFIGVKGSGMGAQGIRKSLESMTREKVTVLNLV

SEQ ID NO: 23 - protein, 478 aa, Bacillus cereus
MMASIYKFYLNGEWRESSSGETIEILSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKASMIPLVLELGGKDPGIVREDADLQDAANHIVSGAFSYSGQRCTAIKRVLVHENVADELVGLLKAQVAKLSVGSPEQDSTIVPLIDDKSADFVQGLVDDAVEKGATIVIGNKRERNLIYPTLIDHVTEDMTVAWEEPFGPILPIIRVSSDEQAIEIANKSEFGLQASVFTKDINKAFAIANKIETGSVQINGRTERGPDHFPFIGVKGSGMGAQGIRKSLESMTREKVTVVNFK

SEQ ID NO: 24 - protein, 479 aa, Bacillus cereus
MTTSNTYKFYLNGEWRESSSGQIIDIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVDLVKAQVAELSVGSPEQDSTIVPLIDDKSADFVQGLVDDAVEKGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 25 - protein, 479 aa, Bacillus cereus
MMMASIYKFYLNGEWRESSSGETIEILSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGIDMVSFTGGTNTGKHLAKKAAMITLVLELGGKDPGIVREDADLQDAANHIVSGAFSYSGQRCTAIKRVLVHENVADELVSLLQEQVAKLSVGSPEQDSTIVPLIDDKSADFVQGLVDDAVEKGATIVIGNKRERNLIYPTLIDHVTEDMTVAWEEPFGPILPIIRVSSDEQAIEIANKSEFGLQASVFTKDINKAFAIANKIETGSVQINGRTERGPDHFPFIGVKGSGMGAQGIRKSLESMTREKVTVVNFK

SEQ ID NO: 26 - protein, 479 aa, Bacillus pseudomycoides
MTTSNTYKFYLDGEWRESSSGQTIDIPSPYLHEVIGKVQAITREEVDEAIKSAQQAQKEWAEASLQDRAKYLYKWADELVNMQDEIADIVMKEVGKGYKDAKKEVVRTADLIRYTVDEALHMHGESMMGDSFPGGSKSKLAIIQRAPRGVVLAIAPFNYPVNLSAAKLAPALIMGNAVIFKPATQGVISGIKMVEALHKAGLPKGLVNVATGRGSVIGDYLVEHPGVNMVSFTGGTHTGAHLAKKAAMIPLVLELGGKDPGIVREDADLGEAAKHIVSGAFSYSGQRCTAIKRVLVHENVADELVSLLKDQVAELTVGSPEQDSTIVPLIDDKSADFVQGLVDDAVEKGATIVIGNKRERNLIYPTLIDNVTEDMKVAWEEPFGPILPIIRVSSDEQAIEIANKSDFGLQASVFTKDINKAFAIANKIDTGSVQINGRTERGPDHFPFIGVKGSGMGAQGIRKSLESMTREKVTVLNLV

SEQ ID NO: 27 - protein, 825 aa, Bifidobacterium animalis
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMNNPSLFVPCIIGDGEAETGPLATGWQSNKLVNPRTDGIVLPILHLNGYKIANPTLLARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKDDVTAFMPKGELRIGANPNANGGRIREDLKLPELDQYEITGVKEYGHGWGQVEAPRSLGAYCRDIIKNNPDSFRIFGPDETASNRLNATYEVTKKQWDNGYLSALVDENMAVTGQVVEQLSEHQCEGFLEAYLLTGRHGMWSTYESFAHVIDSMLNQHAKWLEATVREIPWRKPISSVNLLVSSHVWRQDHNGFSHQDPGVTSVLINKTFNNDHVTNIYFATDANMLLAIAEKCFKSTNKINAIFSGKQPAPTWITLDEARAELEAGAAEWKWASNAKSNDEVQIVLAAAGDVPTQEIMAASDALNKDGIKFKVVNVVDLLKLQSPENNDEAMSNEDFTELFTADKPVLFAYHSYAQDVRGLIYDRPNHDNFNVVGYKEQGSTTTPFDMVRVNDMDRYALEAQALELIDADKYADKIDELNAFRKTAFQFAVDNGYDIPEFTDWVYPDVKVDETQMLSATAATAGDNE

SEQ ID NO: 28 - protein, 813 aa, Leuconostoc mesenteroides
MADFDSKEYLELVDKWWRATNYLSAGMIFLKSNPLFSVTNTPIKAEDVKVKPIGHWGTISGQTFLYAHANRLINKYGLNMFYVGGPGHGGQVMVTNAYLDGAYTEDYPEITQDIEGMSHLFKRFSFPGGIGSHMTAQTPGSLHEGGELGYSLSHAFGAVLDNPDQVAFAVVGDGEAETGPSMASWHSIKFLNAKNDGAVLPVLDLNGFKISNPTIFSRMSDEEITKFFEGLGYSPRFIENDDIHDYATYHQLAANILDQAIEDIQAIQNDARENGKYQDGEIPAWPVIIARLPKGWGGPTHDASNNPIENSFRAHQVPLPLEQHDLATLPEFEDWMNSYKPEELFNADGSLKDELKAIAPKGDKRMSANPITNGGADRSDLKLPNWREFANDINDDTRGKEFADSKRNMDMATLSNYLGAVSQLNPTRFRFFGPDETMSNRLWGLFNVTPRQWMEEIKEPQDQLLSPTGRIIDSQLSEHQAEGWLEGYTLTGRVGIFASYESFLRVVDTMVTQHFKWLRHASEQAWRNDYPSLNLIATSTAFQQDHNGYTHQDPGMLTHLAEKKSNFIREYLPADGNSLLAVQERAFSERHKVNLLIASKQPRQQWFTVEEAEVLANEGLKIIDWASTAPSSDVDITFASAGTEPTIETLAALWLINQAFPDVKFRYVNVVELLRLQKKSEPNMNDERELSAEEFNKYFQADTPVIFGFHAYENLIESFFFERKFTGDVYVHGYREDGDITTTYDMRVYSHLDRFHQAKEAAEILSANGKIDQAAADTFIAKMDDTLAKHFQVTRNEGRDIEEFTDWTWSPLK

SEQ ID NO: 29 - protein, 796 aa, Clostridium acetobutylicum
  source 1..796: note = strain ATCC 824
MQSIIGKHKDEGKITPEYLKKIDAYWRAANFISVGQLYLLDNPLLREPLKPEHLKRKVVGHWGTIPGQNFIYAHLNRVIKKYDLDMIYVSGPGHGGQVMVSNSYLDGTYSEVYPNVSRDLNGLKKLCKQFSFPGGISSHMAPETPGSINEGGELGYSLAHSFGAVFDNPDLITACVVGDGEAETGPLATSWQANKFLNPVTDGAVLPILHLNGYKISNPTVLSRIPKDELEKFFEGNGWKPYFVEGEDPETMHKLMAETLDIVTEEILNIQKNARENNDCSRPKWPMIVLRTPKGWTGPKFVDGVPNEGSFRAHQVPLAVDRYHTENLDQLEEWLKSYKPEELFDENYRLIPELEELTPKGNKRMAANLHANGGLLLRELRTPDFRDYAVDVPTPGSTVKQDMIELGKYVRDVVKLNEDTRNFRIFGPDETMSNRLWAVFEGTKRQWLSEIKEPNDEFLSNDGRIVDSMLSEHLCEGWLEGYLLTGRHGFFASYEAFLRIVDSMITQHGKWLKVTSQLPWRKDIASLNLIATSNVWQQDHNGYTHQDPGLLGHIVDKKPEIVRAYLPADANTLLAVFDKCLHTKHKINLLVTSKHPRQQWLTMDQAVKHVEQGISIWDWASNDKGQEPDVVIASCGDTPTLEALAAVTILHEHLPELKVRFVNVVDMMKLLPENEHPHGLSDKDYNALFTTDKPVIFAFHGFAHLINQLTYHRENRNLHVHGYMEEGTITTPFDMRVQNKLDRFNLVKDVVENLPQLGNRGAHLVQLMNDKLVEHNQYIREVGEDLPEITNWQWHV

SEQ ID NO: 30 - protein, 780 aa, Aspergillus nidulans
MPGEVIERPNPAPKPSHVPDLVEKLIIPAQKTKLEKSDCDALHKYRRAAAYIAAGHWGTCPGLILVYSHLNYLIKKQNLDMLYVVGPGHGAPGLLASLWLEGSLGKFYPQYTKDKEGLHNLISTFSTSAGLPSHINAETPGAIHEGGELGYALSVSFGAVMDNPDLIVTCVVGDGEAETGPTATSWHAIKYIDPAESGAVLPILHVNGFKISERTIFGCMDNREIVCLFTGYGYQVRIVEDLEDIDNDLHSAMSWAVEEIRNIQKAARSGKPIMKPQWPMIVLRTPKGWSGPKELHGQFIEGSFHSHQVPLPNAKKDDEELQALQKWLSSYKPDELFTESGDVIDEILSIIPSDDKKLGMRPEAYKTHLPPDLPDWRQFCVKKGDQFSAMKAIGSFIDQVFVKNPHTVRLFSPDELESNKLSAALSHTGRNFQWDEFSNAKGGRVIEVLSEHLCQGFMQGYTLTGRTGIFPSYESFLGIIHTMMVQYAKFAKMAKETAWHHDVSSINYIETSTWARQEHNGFSHQNPSFIGAVLKLKPYAARVYLPPDANTFLTTLHHCLKSKNYINLMVGSKQPTPVYLSPEEAESHCRAGASIFKFCSTDGGLRPDVVLVGIGVEVMFEVIKAAAILRERCPELRVRVVNVTDLFILENEGAHPHALKHEAFDNLFTEDRSIHFNYHGYVNELQGLLFGRPRLDRATIKGYKEEGSTTTPFDMMLVNEVSRYHVAKAAVTGGARFNEKVKLRHQELCSEFDHNIAETRKYIMNNHQDPEDTYNMPSFN

SEQ ID NO: 31 - protein, 825 aa, Bifidobacterium pseudolongum
  source 1..825: note = sub_species globosum
MTNPVIGTPWQKLDRPVSEEAIEGMDKYWRVTNYMSIGQIYLRSNPLMKEPFTRDDVKHRLVGHWGTTPGLNFLLAHINRLIADHQQNTVFIMGPGHGGPAGTSQSYVDGTYTEYYPNITKDEAGLQKFFRQFSYPGGIPSHFAPETPGSIHEGGELGYALSHAYGAVMNNPSLFVPCIIGDGEAETGPLATGWQSNKLVNPRTDGIVLPILHLNGYKIANPTILARISDEELHDFFRGMGYHPYEFVAGFDNEDHMSIHRRFAELFETIFDEICDIKAAAQTDDMTRPFYPMLIFRTPKGWTCPKFIDGKKTEGSWRAHQVPLASARDTEEHFEVLKGWLESYKPEELFNADGSIKDDVTAFMPKGDLRIGANPNANGGVIREDLKLPELDQYEVTGVKEYGHGWGQVEAPRSLGAYCRDIIKNNPDSFRIFGPDETASNRLNATYEVTDKQWDNGYLSSLVDEHMAVTGQVTEQLSEHQCEGFLEAYLLTGRHGIWSSYESFVHVIDSMLNQHAKWLEATVREIPWRKPISSVNLLVSSHVWRQDHNGFSHQDPGVTSVLINKTFNNDHVTNIYFATDANMLLAISEKCFKSTNKINAIFAGKQPAPTWITLDEARAELEAGAAEWKWASNAENNDEVQVVLAAAGDVPTQEIMAASDALNKMGIKFKVVNVVDLLKLQSRENNDEALTDEEFTDLFTADKPVLFAYHSYAQDVRGLIYDRPNHDNFNVVGYKEQGSTTTPFDMVRVNDMDRYALQAAALKMIDADKYADKIDELNAFRQKAFQFAVDNGYDIPEFTDWVYPDVKVDETQMLSATAATAGDNE

SEQ ID NO: 32 - protein, 825 aa, Bifidobacterium pseudolongum
  source 1..825: note = sub_species globosum
MTNPVIGTPWQKLDRPVSEEAIEGMDKYWRVTNYMSIGQIYLRSNPLMKEPFTRDDVKHRLVGHWGTTPGLNFLLAHINRLIADHQQNTVFIMGPGHGGPAGTAQSYVDGTYTEYYPNITKDEAGLQKFFRQFSYPGGIPSHFAPETPGSIHEGGELGYALSHAYGAVMNNPSLFVPCIIGDGEAETGPLATGWQSNKLVNPRTDGIVLPILHLNGYKIANPTILARISDEELHDFFRGMGYHPYEFVAGFDNEDHMSIHRRFAELFETIFDEICDIKAAAQTDDMTRPFYPMLIFRTPKGWTCPKFIDGKKTEGSWRAHQVPLASARDTEEHFEVLKGWMESYKPEELFNADGSIKDDVTEFMPKGELRIGANPNANGGRIREDLKLPELDQYEVTGVKEYGHGWGQVEAPRSLGAYSRDIIKNNPDSFRIFGPDETASNRLNATYEVTKKQWDNGYLSALVDENMAVTGQVVEQLSEHQCEGWLEGYILTGRHGMWSTYESFAHVIDSMLNQHAKWLEATVREIPWRKPISSVNLLISSHVWRQDHNGFSHQDPGVTSVLINKTFNNDHVTNIYFATDANMLLAIAEKCFKSTNKINAIFSGKQPAPTWITLDEARAELEAGAAEWKWASNAKSNDEVQIVLAAAGDVPTQEIMAASDALNKEGIKFKVVNVVDLLKLQSPENNDEAMSNEDFAELFTADKPVLFAYHSYAQDVRGLIYDRPNHDNFHVVGYKEQGSTTTPFDMVRVNDMDRYALEAQALELIDADKYADKINELNEFRKKAFQFAVDNGYDIPEFTDWVYPDVKVDETQMLSATAATAGDNE

SEQ ID NO: 33 - protein, 825 aa, Bifidobacterium breve
MTNPVIGTPWQKLDRPVSEEAIEGMDKYWRVTNYMSIGQIYLRSNPLMKEPFTRDDVKHRLVGHWGTTPGLNFLLAHINRLIADHQQNTVFIMGPGHGGPAGTSQSYVDGTYTEYYPNITKDEAGLQKFFRQFSYPGGIPSHFAPETPGSIHEGGELGYALSHAYGAVMNNPSLFVPCIIGDGEAETGPLATGWQSNKLVNPRTDGIVLPILHLNGYKIANPTILARISDEELHDFFRGMGYHPYEFVAGFDNEDHMSIHRRFAELFETIFDEICDIKAAAQTDDMTRPFYPMLIFRTPKGWTCPKFIDGKKTEGSWRAHQVPLASARDTEEHFEVLKGWMESYKPEELFNADGSIKDDVTAFMPKGELRIGANPNANGGVIREDLKLPELDQYEVTGVKEYGHGWGQVEAPRALGAYCRDIIKNNPDSFRIFGPDETASNRLNATYEVTDKQWDNGYLSGLVDEHMAVTGQVTEQLSEHQCEGFLEAYLLTGRHGIWSSYESFVHVIDSMLNQHAKWLEATVREIPWRKPISSVNLLVSSHVWRQDHNGFSHQDPGVTSLLINKTFNNDHVTNIYFATDANMLLAISEKCFKSTNKINAIFAGKQPAPTWVTLDEARAELEAGAAEWKWASNAENNDEVQVVLASAGDVPTQELMAASDALNKMGIKFKVVNVVDLLKLQSRENNDEALTDEEFTELFTADKPVLFAYHSYAQDVRGLIYDRPNHDNFHVVGYKEQGSTTTPFDMVRVNDMDRYALQAAALKLIDADKYADKIDELNAFRKKAFQFAVDNGYDIPEFTDWVYPDVKVDETQMLSATAATAGDNE

SEQ ID NO: 34 - protein, 788 aa, Lactobacillus paraplantarum
MTTDYSSPAYLQKVDKYWRAANYLSVGQLYLKDYPLLQQPLKASDVKVHPICHWGTIAGQNSIYAHLNRVINKYGLKMFYVEGPGHGGQVMVSNSYLDGTYTDIYPEITQDVEGMQKLFKQFSFPGGVASHAAPETPGSIHEGGELGYSISHGVGAILDNPDEIAAVVVGDGESETGPLATSWQSTKFINPINDGAVLPILNLNGFKISNPTIFGRTSDAKIKEYFESMSWEPIFVEGDDPEKVHPVLAKAMDEAVEKIKAIQKHARENDDATLPVWPMIVFRAPKGWTGPKSWDGDKIEGSFRAHQIPIPVDQNDMEHADALVDWLESYQPKELFNEDGSLKDDIKEIIPTGDSRMAANPITNGGVDPKALNLPNFRDYAVDTSKEGANVKQDMLVWSDYLRDVIKKNPDNFRLFGPDETMSNRLYGVFETTNRQWMEDIHPDSDQYEAAAGRVLDAQLSEHQAEGWLEGYVLTGRHGLFASYEAFLRVVDSMLTQHFKWLRKANELDWRKKYPSLNIIAASTVFQQDHNGYTHQDPGALTHLAEKKPEYIREYLPADANTLLAVGDVIFRSQEKINYVVTSKHPRQQWFSIEEAKQLVDNGLGIIDWASTDQGSEPDIVFAAAGTEPTLETLAAIQLLHDSFPEMKIRFVNVVDILKLRSPEKDPRGLSDAEFDHYFTKDKPVVFAFHGYEDLVRDIFFDRHNHNLYVHGYRENGDITTPFDVRVMNQMDRFDLAKTAIAAQPAMENTGAAFVQSMDNMLAKHNAYIRDAGTDLPEVNDWQWKGLK